SEQUENCE LISTING (52 sequences)

SEQ ID NO: 1 - protein, 112 aa, Simian immunodeficiency virus
MSDPRERIPPGNSGEETIGEAFEWLNRTVEEINREAVNHLPRELIFQVWQRSWEYWHDEQGMSQSYVKYRYLCLMQKALFMHCKKGCRCLGEGHGAGGWRPGPPPPPPPGLA

SEQ ID NO: 2 - protein, 112 aa, Simian immunodeficiency virus
MTDPRERIPPGNSGEETIGEAFEWLHNTVEALNQTAVQHLPRELIFQVWRRCWEYWVDEQGYSPSYAKYRYVQLMQKAMFQHFRKGCTCRGEGHSQGGWRTGPPPPPPPGLA

SEQ ID NO: 3 - protein, 112 aa, Simian immunodeficiency virus
MSDPRERIPPGNSGEETIGEAFDWLDRTVEEINRAAVNHLPRELIFQVWRRSWEYWHDEMGMSVSYTKYRYLCLIQKAMFMHCKKGCRCLGGEHGAGGWRPGPPPPPPPGLA

SEQ ID NO: 4 - protein, 112 aa, Simian immunodeficiency virus
MADPRERVPPGNSGEETIGEAFEWLDRTIEALNREAVNHLPRELIFQVWQRSWAYWHDEQGMSTSYTKYRYLCIMQKAVYIHFKKGCTCLGRGHGPGGWRPGPPPPPPPGLV

SEQ ID NO: 5 - protein, 117 aa, Simian immunodeficiency virus
MERYPPSHPPHFTSRTVPMTRLALQQAMQDLNEEALKHFTREELWGVWNHCVDLPAQPDWTGEQAWAASVIDYIKIVQRMLWLHLREACFHREREATRRYPNIRPLTGRNREVRDGE

SEQ ID NO: 6 - protein, 135 aa, Simian immunodeficiency virus
MERVPPSHRPPWHSRVVPTTMQQAQQAMWDLNEEAEKHFSREELRGIWNDVTELPADPNWTVDQAAIACAIDYIRRTQTLLFRHYREGCYHRYSNTIRRYPNIRPLRGTQAPPSNSMPNADPTPPLRPSRYRMDE

SEQ ID NO: 7 - protein, 119 aa, Simian immunodeficiency virus
MASGRDPREERPGELEIWDLSREPWDEWLRDMLTELNQEAQRHFGRELLFQVWNYCQEEGERQNVPMQERAYKYYKLVQRALFVHFRCGCRRRQPFEPYEERRNGQGGGRAGRVPPGLD

SEQ ID NO: 8 - protein, 119 aa, Simian immunodeficiency virus
MASGRDPREERPGEVEIWDLSREPWDEWLRDMLAELNREAQRHFGRELLFQVWNYCQEEGERQNIPMQERAYKYYRLVQKALFVHIRCGCRRRQPFEPYEERRNGQGGGRAERVPPGLD

SEQ ID NO: 9 - protein, 117 aa, Simian immunodeficiency virus; identical to SEQ ID NO: 5

SEQ ID NO: 10 - protein, 113 aa, Simian immunodeficiency virus
MERLPPSHPPALVSRMAETTQRQLQEAVWDITEEARKHFSKEEITGIWDHCWSLPALPHWTEGQVMAAAVIDFIRIMQKEIWKHYRVGCFHREAERVRHYPNIRPLRPRREEP

SEQ ID NO: 11 - protein, 115 aa, Simian immunodeficiency virus
MERLPPSHPLPLTSRLIPTPRPQLERLMREVRDEMLIHFTRDEVIGVWNHCVELPADPDWTGEQAWAASVIDFARKGNQMLLLHFQQGCYHEQQRRTPHYPNIRPLSGRGERTMQ

SEQ ID NO: 12 - protein, 133 aa, Simian immunodeficiency virus
MERIPPSHPMPWLSRRVPTTMAIAQNALWEINEEAEKHFSRDELRGIWHDVTELPADPDWTVDQAAIACAIDYIRVQTLLFRHFKDGCFHRVNLSGRYPAIRPSRGTAPPDSNSVSHADPEQPRRPSRYRMDE

SEQ ID NO: 13 - protein, 135 aa, Simian immunodeficiency virus
MERTPPSHPLPWISRRVPTTMAVAQSAMWEINEEAEKHFSREELRGIWHDVTELPADPDWTVDQAAIACAIDYVRRVQTLLFRHFRDGCFHRYNRIVRRYPVIRPLRGTAPPDSNSVPHADPEQPRRPSRYRMDE

SEQ ID NO: 14 - protein, 135 aa, Simian immunodeficiency virus
MEQPPQSHPLHWTSRMVPIERQALQAAIWELNEEALKHFSREELRGIWEQVTELPADPAWNADQAWAACAIDYTRWVQTILYRHYREGCYHRYAEQIRRYPVLRPMRGTAPGPTSSVPQADPDNPRRPSRYRMDE

SEQ ID NO: 15 - protein, 113 aa, Simian immunodeficiency virus
MAEAFFNPSQHVQGTPWFFIPRNVELTPNVINVTVKAELVVTEASKHFTPQEIYGVWNQSLNEEAGTDSPTMAWERTMLDMVRALNLMLFEHFAAGCPQRTRYARHRGYPHPS

SEQ ID NO: 16 - protein, 99 aa, Simian immunodeficiency virus
MAERAPEAPEGAGEVGLEQWLETSLERINREARLHFHPEFLFRLWNTCVEHWHDRHQRSLDYAKYRYLLLMHKAMYTHMQQGCPCRNGRPRGPPPPGMA

SEQ ID NO: 17 - protein, 113 aa, Simian immunodeficiency virus
MAERQSVERAPAEPMGAGEVELEEWLQRSLLRINQEARLHFHPEFLFRLWNTCMEHYHDALQLSFTYSKYRYLLLLQKAMFMHFQQGCSCLQGRHPPPLRPAGDRLPPPPPPP

SEQ ID NO: 18 - protein, 112 aa, Simian immunodeficiency virus
MSDPRERIPPGNSGEETIGEAFEWLNRTVEEINREAVNHLPRELIFQVWQRSWEYWHDEQGMSPSYVKYRYLCLIQKALFMHCKKGCRCLGEGHGAGGWRPGPPPPPPPGLA

SEQ ID NO: 19 - protein, 626 aa, Homo sapiens
  MISC_FEATURE: Human SAMHD1
MQRADSEQPSKRPRCDDSPRTPSNTPSAEADWSPGLELHPDYKTWGPEQVCSFLRRGGFEEPVLLKNIRENEITGALLPCLDESRFENLGVSSLGERKKLLSYIQRLVQIHVDTMKVINDPIHGHIELHPLLVRIIDTPQFQRLRYIKQLGGGYYVFPGASHNRFEHSLGVGYLAGCLVHALGEKQPELQISERDVLCVQIAGLCHDLGHGPFSHMFDGRFIPLARPEVKWTHEQGSVMMFEHLINSNGIKPVMEQYGLIPEEDICFIKEQIVGPLESPVEDSLWPYKGRPENKSFLYEIVSNKRNGIDVDKWDYFARDCHHLGIQNNFDYKRFIKFARVCEVDNELRICARDKEVGNLYDMFHTRNSLHRRAYQHKVGNIIDTMITDAFLKADDYIEITGAGGKKYRISTAIDDMEAYTKLTDNIFLEILYSTDPKLKDAREILKQIEYRNLFKYVGETQPTGQIKIKREDYESLPKEVASAKPKVLLDVKLKAEDFIVDVINMDYGMQEKNPIDHVSFYCKTAPNRAIRITKNQVSQLLPEKFAEQLIRVYCKKVDRKSLYAARQYFVQWCADRNFTKPQDGDVIAPLITPQKKEWNDSTSVQNPTRLREASKSRVQLFKDDPM

SEQ ID NO: 20 - protein, 52 aa, Human immunodeficiency virus
  MISC_FEATURE: HIV-1 P6
LQSRPEPTAPPEESFRFGVETTTPPQKQEPIDKELYPLTSLRSLFGNDPSSQ

SEQ ID NO: 21 - protein, 63 aa, Simian immunodeficiency virus
  MISC_FEATURE: SIVmac P6
PMAQVHQGLMPTAPPEDPAVDLLKNYMQLGKQQREKQRESREKPYKEVTEDLLHLNSLFGGDQ

SEQ ID NO: 22 - protein, 7 aa, Artificial Sequence
  synthetic construct
  MISC_FEATURE: SIVmac P6
  VARIANT 2, 4, 5: Xaa can be any amino acid
  misc_feature (4)..(5): Xaa can be any naturally occurring amino acid
DXAXXLL

SEQ ID NO: 23 - protein, 7 aa, Artificial Sequence
  synthetic construct
  MISC_FEATURE: packaging motif
DPAVDLL

SEQ ID NO: 24 - protein, 10 aa, Artificial Sequence
  synthetic construct
  MISC_FEATURE: packaging motif
DPAVDLLKNY

SEQ ID NO: 25 - protein, 52 aa, Artificial Sequence
  synthetic construc
  MISC_FEATURE: Hybrid P6 domain
LQSRPEPTAPPEESDPAVDLLTTPPQKQEPIDKELYPLTSLRSLFGNDPSSQ

SEQ ID NO: 26 - protein, 16 aa, Artificial Sequence
  synthetic construct
  MISC_FEATURE: Binding motif
  VARIANT 6, 9, 14: Xaa can be Val, Ile, Leu, Phe, Trp, Tyr or Met
  VARIANT 2, 3, 4, 5, 7, 8, 10, 11, 12, 15: Xaa can be any amino acid
WXXXXXXXXXXXAXXH

SEQ ID NO: 27 - protein, 12 aa, Artificial Sequence
  spacer (IgG4hinge) (aa)
ESKYGPPCPPCP

SEQ ID NO: 28 - protein, 36 aa, Artificial Sequence
  spacer (IgG4hinge) (nt)
GAATCTAAGTACGGACCGCCCTGCCCCCCTTGCCCT

SEQ ID NO: 29 - protein, 119 aa, Artificial Sequence
  Hinge-CH3 spacer
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 229 aa, Artificial Sequence
  Hinge-CH2-CH3 spacer
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 282 aa, Artificial Sequence
  IgD-hinge-Fc
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 32 - protein, 24 aa, Artificial Sequence
  T2A
LEGGGEGRGSLLTCGDVEENPGPR

SEQ ID NO: 33 - protein, 357 aa, Artificial Sequence
  tEGFR
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 34 - protein, 27 aa, Homo sapiens
  MISC_FEATURE: CD28 (amino acids 153-179 of Accession No. P10747)
FWVLVVVGGVLACYSLLVTVAFIIFWV

SEQ ID NO: 35 - protein, 66 aa, Homo sapiens
  MISC_FEATURE: CD28 (amino acids 114-179 of Accession No. P10747)
IEVMYPPPYLDNEKSNGTIIHVKGKHLCPSPLFPGPSKPFWVLVVVGGVLACYSLLVTVAFIIFWV

SEQ ID NO: 36 - protein, 41 aa, Homo sapiens
  MISC_FEATURE: CD28 (amino acids 180-220 of P10747)
RSKRSRLLHSDYMNMTPRRPGPTRKHYQPYAPPRDFAAYRS

SEQ ID NO: 37 - protein, 41 aa, Homo sapiens
  MISC_FEATURE: CD28 (LL to GG)
RSKRSRGGHSDYMNMTPRRPGPTRKHYQPYAPPRDFAAYRS

SEQ ID NO: 38 - protein, 42 aa, Homo sapiens
  MISC_FEATURE: 4-1BB (amino acids 214-255 of Q07011.1)
KRGRKKLLYIFKQPFMRPVQTTQEEDGCSCRFPEEEEGGCEL

SEQ ID NO: 39 - protein, 112 aa, Homo sapiens
  MISC_FEATURE: CD3 zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 112 aa, Homo sapiens
  MISC_FEATURE: CD3 zeta
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 112 aa, Homo sapiens
  MISC_FEATURE: CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 335 aa, Artificial Sequence
  tEGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 43 - protein, 18 aa, Artificial Sequence
  T2A
EGRGSLLTCGDVEENPGP

SEQ ID NO: 44 - protein, 22 aa, Artificial Sequence
  P2A
GSGATNFSLLKQAGDVEENPGP

SEQ ID NO: 45 - protein, 19 aa, Artificial Sequence
  P2A
ATNFSLLKQAGDVEENPGP

SEQ ID NO: 46 - protein, 20 aa, Artificial Sequence
  E2A
QCTNYALLKLAGDVESNPGP

SEQ ID NO: 47 - protein, 22 aa, Artificial Sequence
  F2A
VKQTLNFDLLKLAGDVESNPGP

SEQ ID NO: 48 - protein, 30 aa, Artificial Sequence
  Linker
PGGGSGGGGSGGGGSGGGGSGGGGSGGGGP

SEQ ID NO: 49 - protein, 17 aa, Artificial Sequence
  Linker
GSADDAKKDAAKKDGKS

SEQ ID NO: 50 - DNA, 66 bp, Artificial Sequence
  GMCSFR alpha chain signal sequence
atgcttctcctggtgacaagccttctgctctgtgagttaccacacccagcattcctcctgatccca

SEQ ID NO: 51 - protein, 22 aa, Artificial Sequence
  GMCSFR alpha chain signal sequence
MLLLVTSLLLCELPHPAFLLIP

SEQ ID NO: 52 - protein, 18 aa, Artificial Sequence
  CD8 alpha signal peptide
MALPVTALLLPLALLLHA

(19) United States
(12) Patent Application Publication
(10) Pub. No.: US 2019/0350978 A1
(43) Pub. Date: Nov. 21, 2019

(54) PRODUCTION OF ENGINEERED CELLS FOR ADOPTIVE CELL THERAPY

(71) Applicant: Juno Therapeutics, Inc., Seattle, WA (US)

(72) Inventors: Pascal BEAUCHESNE, Seattle, WA (US); Semih U. TAREEN, Seattle, WA (US)

(73) Assignee: Juno Therapeutics, Inc., Seattle, WA (US)

(21) Appl. No.: 16/465,140

(22) PCT Filed: Dec. 5, 2017
(86) PCT No.: PCT/US2017/064778

§ 371 Date: May 29, 2019

Related U.S. Application Data
(60) Provisional application No. 62/430,349, filed on Dec. 5, 2016.

(51) Int. Cl. A61K 35/17; C12N 5/0783; C07K 14/725; C07K 14/47

(52) U.S. Cl. CPC A61K 35/17; C12N 5/0636; C07K 14/7051; C07K 14/4748; C12N 2740/15041; A61K 45/06; C12N 2501/51; C12N 2501/2302; C12N 2501/2307; C12N 2501/2315; C12N 2501/515

(57) ABSTRACT

Provided are methods for genetically engineering cells, including cells for use in connection with genetic engineering. In some embodiments, the provided methods including transduction of cells by incubation with a retroviral vector particle, e.g. lentiviral vector, in which, prior to the incubation, the cells have not been incubated with an activating or stimulating agent, such as have not been incubated with anti-CD3/anti-CD28 antibodies and/or one or more recombinant cytokines. In some embodiments, such methods result in features related to shortening or improving the process for genetically engineering cells. Also provided are resulting cells, transduced with a recombinant or heterologous gene, such as one encoding a chimeric receptor such as a chimeric antigen receptor, or other recombinant antigen receptor such as a transgenic T cell receptor, and compositions thereof. In some embodiments, the provided cells and compositions can be used in methods of adoptive immunotherapy.

### CROSS-REFERENCE TO RELATED APPLICATIONS

This application claims priority from U.S. provisional application No. 62/430,349, filed on Dec. 5, 2016, entitled “Production of Engineered Cells for Adoptive Cell Therapy,” the contents of which are incorporated by reference in their entirety.

### INCORPORATION BY REFERENCE OF SEQUENCE LISTING

The content of the following submission on ASCII text file is incorporated herein by reference in its entirety: a computer readable form (CRF) of the Sequence Listing (file name: 735042005040SeqList.txt, date created: Nov. 28, 2017, size: 50,233 bytes).

### FIELD

The present disclosure provides methods for genetically engineering cells, including cells for use in connection with adoptive cell therapy. In some embodiments, the provided methods including transduction of cells by incubation with a retroviral vector particle, e.g. lentiviral vector, in which, prior to the incubation, the cells have not been incubated with an activating or stimulating agent, such as have not been incubated with anti-CD3/anti-CD28 antibodies and/or one or more recombinant cytokines. In some embodiments, such methods result in features related to shortening or improving the process for genetically engineering cells. Also provided are resulting cells, transduced with a recombinant or heterologous gene, such as one encoding a chimeric receptor such as a chimeric antigen receptor, or other recombinant antigen receptor such as a transgenic T cell receptor, and compositions thereof. In some embodiments, the provided cells and compositions can be used in methods of adoptive immunotherapy.

### BACKGROUND

Various strategies are available for transducing T cell populations in vitro, including for transducing antigen-specific T cells in vitro for use in adoptive cellular immunotherapy or cancer therapy. Improved strategies are needed for transducing cell populations in vitro, including for research, diagnostic and therapeutic purposes. Provided are reagents, methods, articles of manufacture and kits that meet such needs.

### SUMMARY

Provided herein is a method for transducing T cells including incubating a viral vector particle containing a recombinant nucleic acid and an input composition containing a plurality of T cells, said plurality of T cells having been obtained from a sample containing cells derived from a subject, wherein the incubating is initiated no more than 24 hours after obtaining the sample from the subject; and/or prior to the incubating, the T cells have not been subjected to a temperature greater than or greater than about 15° C., about 18° C., about 22° C. or about 25° C. for a duration of more than 1 hour, 2 hours, 4 hours, 6 hours, 8 hours, 12 hours, or 24 hours after obtaining the sample from the subject; and/or prior to the incubating, the T cells have not been subjected to a temperature of, of about, greater than, or greater than about 37°±2.0° C. for a duration of more than 15 minutes, 30 minutes, 1 hour or 2 hours after obtaining the sample from the subject. In some embodiments, the incubating is initiated no more than or no more than about 1 hour, 3 hours, 6 hours, 12 hours or 18 hours after obtaining the sample from the subject.

In some of any such embodiments, prior to said incubation, the method does not include stimulating the T cells under conditions that promote cell activation. In some of any such embodiments, prior to said incubating, the input composition has not been subjected to an ex vivo stimulation including incubation greater than or greater than about 37° 2.0° C. and/or incubation in the presence of an agent or agents capable of activating T cells, CD4+ T cells, and/or CD8+ T cells, incubation in the presence of an agent or agents capable of inducing a signal through a TCR complex and/or incubation in the presence of an agent or agents capable of inducing proliferation of T cells, CD4+ T cells, and/or CD8+ T cells; CD3-binding molecules; CD28-binding molecules; recombinant IL-2; recombinant IL-15; and recombinant IL-7.

In some of any such embodiments, prior to said incubating, no more than 5%, 10%, 20%, 30%, or 40% of the T cells are activated cells, express a surface marker selected from the group consisting of HLA-DR, CD25, CD69, CD71, CD40L and 4-1BB; include intracellular expression of a cytokine selected from the group consisting of IL-2, IFN-gamma, TNF-alpha, are in the G1 or later phase of the cell cycle and/or are capable of proliferating.

In some of any such embodiments, the method includes incubating a viral vector particle containing a recombinant nucleic acid and an input composition containing T cells, said T cells having been obtained from a sample from a subject, wherein, prior to said incubating, the T cells or input composition have not been subjected to an ex vivo stimulation including incubation greater than or greater than about 37°±2.0° C. and/or incubation in the presence of an agent or agents capable of activating T cells, CD4+ T cells, and/or CD8+ T cells, incubation in the presence of an agent or agents capable of inducing a signal through a TCR complex and/or incubation in the presence of an agent or agents capable of inducing proliferation of T cells, CD4+ T cells, and/or CD8+ T cells; CD3-binding molecules; CD28-binding molecules; recombinant IL-2; recombinant IL-15; and recombinant IL-7.

In some of any such embodiments, the one or more agents contain an anti-CD3 antibody and/or an anti-CD28 antibody.

Also provided is a method for transducing T cells including incubating a viral vector particle containing a recombinant nucleic acid and an input composition containing T cells, said T cells having been obtained from a sample from a subject, wherein, prior to the incubation, no more than 5%, 10%, 20%, 30%, or 40% of the T cells are activated cells, express a surface marker selected from the group consisting of HLA-DR, CD25, CD69, CD71, CD40L and 4-1BB; include intracellular expression of a cytokine selected from the group consisting of IL-2, IFN-gamma, TNF-alpha, and/or are in the G1 or later phase of the cell cycle.

In some of any such embodiments no more than 10% of T cells in the input composition contain a T cell activation marker selected from the group consisting of HLA-DR, CD25, CD69, CD71, CD40L, and 4-1BB immediately prior to the incubation. In some of any such embodiments, prior to said incubating, greater than 5%, 10%, 20%, 30%, or 40% of the T cells express the low-density lipid receptor (LDL-R).

In some of any such embodiments, the subject is a human.

In some of any such embodiments, the T cells have not been and/or are not maintained at a temperature of from 2° C. to 8° C. for more than 48 hours prior to the incubating.

In some of any such embodiments, the sample is a blood sample. In some of any such embodiments, the sample is a leukapheresis sample.

In some of any such embodiments, the T cells are unfractionated T cells, are enriched or isolated CD3+ T cells, are enriched or isolated CD4+ T cells or are enriched or isolated CD8+ T cells. In some of any such embodiments, the T cells have been selected or enriched from the sample from the subject, which, in some aspects, generates an enriched composition and/or generates the input composition.

In some of any such embodiments, the method further includes prior to the incubation, obtaining the sample from the subject and, optionally, selecting or enriching the T cells from the sample, which, in some aspects, generates an enriched composition and/or generates the input composition. In some cases, the percentage of T cells in the input composition is greater than or greater than about 75%, 80%, 85%, 90%, 95% T cells.

In some of any such embodiments, the T cells contain CD4+ or CD8+ cells. In some embodiments, the T cells contain CD4+ and CD8+ cells. In some cases, the ratio of the CD4+ cells to the CD8+ cells is or is about 1:1, 1:2, 2:1, 1:3 or 3:1.

In some of any such embodiments, the sample contains serum or plasma at a concentration of at least or at least about 10% (v/v), at least or at least about 15% (v/v), at least or at least about 20% (v/v), at least or at least about 25% (v/v), at least or at least about 30% (v/v), at least or at least about 33% (v/v), at least or at least about 35% (v/v), or at least or at least about 40% (v/v); and/or prior to the incubating, the sample has been contacted ex vivo with serum or plasma at a concentration of at least or at least about 10% (v/v), at least or at least about 15% (v/v), at least or at least about 20% (v/v), at least or at least about 25% (v/v), at least or at least about 30% (v/v), at least or at least about 33% (v/v), at least or at least about 35% (v/v), or at least or at least about 40% (v/v).

In some of any such embodiments, the sample contains serum or plasma at a concentration of at least or at least about 30% (v/v); and/or prior to the incubating, the sample has been contacted ex vivo with serum or plasma at a concentration of at least or at least about 30% (v/v). In some aspects, the serum or plasma is human. In some cases, the serum or plasma is autologous to the subject.

In some of any such embodiments, the sample contains an anticoagulant and/or prior to the incubating, an anticoagulant has been added to the sample. In some instances, the anticoagulant contains free citrate ion.

In some of any such embodiments, prior to the incubating, the method includes cryopreserving the T cells, optionally T cells of the sample or of the enriched composition, in the presence of a cryoprotectant, thereby producing a cryopreserved composition. In some aspects, prior to the incubating, the method includes washing the cryopreserved composition under conditions to reduce or remove the cyroprotectant and/or to generate the input composition.

In some of any such embodiments, the input composition contains N-acetylcysteine (NAC); serum, optionally human serum; recombinant interleukin-2 (IL-2), recombinant interleukin-15 (IL-15), and/or recombinant interleukin-7 (IL-7).

In some of any such embodiments, the input composition contains N-acetylcysteine at a concentration from or from about 0.4 mg/mL to 4 mg/mL, 0.8 mg/mL to 3.6 mg/mL or 1.6 mg/mL to 2.4 mg/mL, each inclusive; or the input composition contains N-acetylcysteine at a concentration of at least or at least about or about 0.4 mg/mL, 0.8 mg/mL, 1.2 mg/mL, 1.6 mg/mL, 2.0 mg/mL, 2.4 mg/mL, 2.8 mg/mL, 3.2 mg/mL, 3.6 mg/mL or 4.0 mg/mL. In some of any such embodiments, the input composition contains serum, optionally human serum, at a concentration from or from about 0.5% to 25% (v/v), 1.0% to 10% (v/v) or 2.5% to 5.0% (v/v), each inclusive; or the input composition contains serum, optionally human serum, at a concentration of at least or at least about or about 0.5%, 1.%, 2.5%, 5% (v/v) or 10%.

In some of any such embodiments, the input composition contains recombinant IL-2, optionally recombinant human IL-2, at a concentration from or from about 10 IU/mL to 500 IU/mL, 50 IU/mL to 250 IU/mL or 100 IU/mL to 200 IU/mL, each inclusive; or at a concentration of at least or at least about 10 IU/mL, 50 IU/mL, 100 IU/mL, 200 IU/mL, 300 IU/mL, 400 IU/mL or 500 IU/mL; and/or the input composition contains recombinant IL-15, optionally recombinant human IL-15, at a concentration from or from about 1 IU/mL to 100 IU/mL, 2 IU/mL to 50 IU/mL or 5 IU/mL to 10 IU/mL, each inclusive; or at a concentration of at least or at least about 1 IU/mL, 2 IU/mL, 5 IU/mL, 10 IU/mL, 25 IU/mL or 50 IU/mL; and/or the input composition contains recombinant IL-7, optionally recombinant human IL-7, at a concentration from or from about 50 IU/mL to 1500 IU/mL, 100 IU/mL to 1000 IU/mL to 200 IU/mL to 600 IU/mL, each inclusive; or at a concentration of at least or at least about 50 IU/mL, 100 IU/mL, 200 IU/mL, 300 IU/mL, 400 IU/mL, 500 IU/mL, 600 IU/mL, 700 IU/mL, 800 IU/mL, 900 IU/mL or 1000 IU/mL.

In some of any such embodiments, the incubating includes a step of spinoculating the viral vector particles with the input composition. In some instances, spinoculating includes rotating, in an internal cavity of a centrifugal chamber, the viral vector particles and input composition, wherein the rotation is at a relative centrifugal force at an internal surface of the side wall of the cavity that is between or between about 500 g and 2500 g, 500 g and 2000 g 500 g and 1600 g, 500 g an 1000 g, 600 g and 1600 g, 600 g and 1000 g, 1000 g and 2000 g or 1000 g and 1600 g, each inclusive; or at least or at least about 600 g, 800 g, 1000 g, 1200 g, 1600 g, or 2000 g.

In some embodiments, the spinoculating is for a time that is greater than or about 5 minutes, greater than or about 10 minutes, greater than or about 15 minutes, greater than or about 20 minutes, greater than or about 30 minutes, greater than or about 45 minutes, greater than or about 60 minutes, greater than or about 90 minutes or greater than or about 120 minutes; or between or between about 5 minutes and 60 minutes, 10 minutes and 60 minutes, 15 minutes and 60 minutes, 15 minutes and 45 minutes, 30 minutes and 60 minutes or 45 minutes and 60 minutes, each inclusive.

In some of any such embodiments, the method further includes contacting the input composition and/or viral vector particles with a transduction adjuvant. In some cases, the contacting is carried out prior to, concomitant with, or after spinoculating the viral vector particles with the input composition.

In some of any such embodiments, at least a portion of the incubation is carried out at or about 37° C.±2° C. In some aspects, the at least a portion of the incubation is carried out after the spinoculation. In some cases, the at least a portion of the incubation is carried out for no more than or no more than about 2 hours, 4 hours, 12 hours, 18 hours, 24 hours, 30 hours, 36 hours, 48 hours, 60 hours or 72 hours. In some embodiments, the at least a portion of the incubation is carried out for or for about 24 hours. In some of any such embodiments, the total duration of the incubation is for no more than 12 hours, 24 hours, 36 hours, 48 hours or 72 hours.

In some of any such embodiments, the viral vector particle is a lentiviral vector particle. In some cases, the lentiviral vector particle is derived from HIV-1. In some of any such embodiments, the viral vector particle is pseudotyped with a viral envelope glycoprotein. In some cases, the viral envelope glycoprotein is VSV-G.

In some of any such embodiments, the viral vector particle contains a lentiviral protein that exhibits SAMHD1-inhibiting activity, said protein being packaged in the viral particle. In some instances, the SAMHD1-inhibiting protein is a wild-type Vpx protein, a wild-type Vpr protein, or is a variant or portion of a wild-type Vpx or Vpr protein that exhibits SAMHD1-inhibiting activity. In some cases, the SAMHD1-inhibiting protein is heterologous to the retroviral vector particle. In some embodiments, the SAMHD1-inhibiting protein is a wild-type Vpx protein or is a variant or portion of a wild-type Vpx protein that exhibits SAMHD1-inhibiting activity.

In some of any such embodiments, the viral vector particle is incubated at a multiplicity of infection of less than or less than about 20.0 or less than or less than about 10.0. In some of any such embodiments, the viral vector particle is incubated at a multiplicity of infection from or from about 1.0 IU/cell to 10 IU/cell or 2.0 U/cell to 5.0 IU/cell; or the viral vector particle is incubated at a multiplicity of infection of at least or at least about 1.6 IU/cell, 1.8 IU/cell, 2.0 IU/cell, 2.4 IU/cell, 2.8 IU/cell, 3.2 IU/cell or 3.6 IU/cell, 4.0 IU/cell, 5.0 IU/cell, 6.0 IU/cell, 7.0 IU/cell, 8.0 IU/cell, 9.0 IU/cell or 10.0 IU/cell.

In some of any such embodiments, the input composition contains at least at or about at least or about 50×10⁶ cells, 100×10⁶ cells, or 200×10⁶ cells.

In some of any such embodiments, the recombinant nucleic acid encodes an antigen receptor. In some cases, the antigen receptor is a transgenic T cell receptor (TCR). In some embodiments, the antigen receptor is a chimeric antigen receptor (CAR). In some instances, the chimeric antigen receptor (CAR) contains an extracellular antigen-recognition domain that specifically binds to a target antigen and an intracellular signaling domain containing an ITAM. In some aspects, the intracellular signaling domain contains an intracellular domain of a CD3-zeta (CD3) chain. In some of any such embodiments, the CAR further contains a transmembrane domain linking the extracellular domain and the intracellular signaling domain. In some cases, the transmembrane domain contains a transmembrane portion of CD28.

In some of any such embodiments, the intracellular signaling domain further contains an intracellular signaling domain of a T cell costimulatory molecule. In some instances, the T cell costimulatory molecule is selected from the group consisting of CD28 and 41BB.

In some of any such embodiments, the antigen receptor specifically binds to an antigen associated with a disease or condition or specifically binds to a universal tag. In some cases, the disease or condition is a cancer, and autoimmune disease or disorder, or an infectious disease.

In some of any such embodiments, the method produces an output composition containing T cells transduced with the recombinant nucleic acid. In some cases, at least 30%, or at least 40%, at least 50%, at least 60%, at least 70% or at least 80% of the T cells in the output composition are transduced with the recombinant nucleic acid. In some aspects, the method further includes recovering or isolating from the output composition the transduced T cells produced by the method. In some cases, the method further includes activating or expanding the cells of the output composition or the cells transduced by the method. In some instances, activation and/or expansion is performed ex vivo.

In some of any such embodiments, subsequent to the incubation, the cells in the output composition are further incubated in the presence of one or more stimulating agent capable of activating T cells, inducing a signal through a TCR complex and/or inducing proliferation of T cells. In some aspects, the one or more stimulating agent is selected from the group consisting of CD3-binding molecules; CD28-binding molecules; recombinant IL-2; recombinant IL-15; and recombinant IL-7. In some embodiments, the one or more stimulating agents contain an anti-CD3 antibody and/or an anti-CD28 antibody.

In some cases, activation and/or expansion is performed in vivo. In some embodiments, activation and/or expansion occurs in the presence of antigen specifically bound by the antigen receptor and/or is transgene-specific.

In some of any such embodiments, subsequent to the incubation, the cells in the output composition are not further incubated ex vivo in the presence of one or more stimulating agent, optionally consisting of CD3-binding molecules; CD28-binding molecules; recombinant IL-2; recombinant IL-15; and recombinant IL-7, and/or the cells in the output composition are not further incubated at a temperature greater than 30° C. for more than 24 hours.

Also provided is a genetically engineered T cell produced by any of the method as described herein. Also provided is a composition containing the genetically engineered T cell of as described herein and a pharmaceutically acceptable carrier.

Provided also is a method of treatment including administering to a subject having a disease or condition the composition as described above. In some cases, the composition is administered or is ready to be administered to the subject, and/or is released for testing no more than 9 days, no more than 8 days, no more than 7 days, no more than 6 days or no more than 5 days after obtaining the sample from the subject. In some instances, the composition is administered or is ready to be administered to the subject and/or is released for testing no more than 1 day, 2 days, 3 days or 4 days after obtaining the sample from the subject. In some cases, the composition is ready to be administered to the subject no more than 21 days, no more than 20 days, no more than 19 days, no more than 15 days, no more than 14 days, no more than 13 days, no more than 12 days, no more than 10 days, no more than 9 days, no more than 8 days, no more than 7 days, no more than 6 days or no more than 5 days after obtaining the sample from the subject.

Also provided is a method for adoptive cell therapy including enriching or isolating T cells from a sample obtained from subject having a disease or condition; transducing an input composition containing the enriched or isolated T cells with a viral vector particle by any of the methods described above, thereby producing an output composition containing transduced cells, wherein the viral vector particle contains a recombinant nucleic acid encoding an antigen receptor that specifically binds to an antigen associated with the disease or disorder; and administering the output composition containing the transduced cells to the subject for treating the disease or condition.

Also provided is a method of treatment that includes administering an output composition comprising T cell transduced with a recombinant nucleic acid to a subject for treating a disease or condition, wherein the output composition is produced by any of the provided methods of transducing cells.

In embodiment of any such methods the composition is administered or is ready to be administered to the subject, or is released for testing no more than 11 days, no more than 9 days, no more than 8 days, no more than 7 days, no more than 6 days or no more than 5 days after obtaining the sample from the subject. In some embodiments, the composition is administered or is ready to be administered to the subject no more than 1 day, 2 days, 3 days, 4 days or 5 days after obtaining the sample from the subject. In some cases, prior to administering the composition, the transduced cells or the cells of the output composition are formulated in a pharmaceutically acceptable buffer.

In some of any such embodiments, prior to administering the output composition containing transduced cells, the cells are cultured ex vivo for up to 24 hours, 2 days, 3 days, 4 days, 5 days, 6 days, 7 days, 8 days, 9 days, or 10 days after the transducing, said culturing occurring at a temperature greater than 30° C. In some embodiments, subsequent to the transduction, the output composition or cells containing the transduced cells are cultured in the presence of one or more stimulating agent capable of activating T cells, inducing a signal through a TCR complex and/or inducing proliferation of T cells, thereby producing the composition containing the transduced cells.

In some of any such embodiments, prior to administering the transduced cells, the output composition or cells containing the transduced cells are not further incubated ex vivo in the presence of one or more stimulating agent and/or are not further incubated at a temperature greater than 30° C. for more than 24 hours.

In some of any such embodiments, the one or more stimulating agent is selected from the group consisting of CD3-binding molecules; CD28-binding molecules; recombinant IL-2; recombinant IL-15; and recombinant IL-7, a vaccine comprising an antigen specifically recognized by the antigen receptor, and an anti-idiotype antibody that specifically binds the antigen receptor.

In some cases, the one or more stimulating agents contain an anti-CD3 antibody and/or an anti-CD28 antibody. In some embodiments, the cells of the output composition or the transduced cells are administered at a sub-optimal dose.

In some of any such embodiments, the method further includes administering to the subject one or more agents to induce or enhance stimulation and/or expansion of the transduced T cells in vivo. In some cases, the one or more agents is transgene-specific and/or stimulates or activates the cells via the expressed transgene, which optionally is or contains an antigen receptor. In some aspects, the one or more agent is selected from among a vaccine containing an antigen specifically recognized by the antigen receptor, an anti-idiotype antibody that specifically binds the antigen receptor or an agent capable of chemically inducing dimerization of the antigen receptor. In some embodiments, the one or more agents is an immunomodulatory agent; an immune checkpoint inhibitor; an inhibitor of extracellular adenosine or adenosine receptor, optionally an A2aR receptor; a kynurenine pathway modulator, and modulators of signaling pathways, e.g., kinase inhibitors.

Also provided is a composition containing a population of primary human T cells genetically engineered to express a chimeric antigen receptor (CAR) or transgenic TCR that specifically binds to a target antigen, wherein the population contains a plurality of resting T cells; and the plurality of resting T cells contain at least 7.5% percent of the genetically engineered cells in the composition. In some instances, the genetically engineered resting T cells contain at least 10%, at least 20%, at least 30%, at least 40%, at least 50%, at least 60%, at least 70%, at least 80%, or at least 90% of the genetically engineered cells in the composition.

In some embodiments, the resting T cells are surface negative for a T cell activation marker selected from the group consisting of HLA-DR, CD25, CD69, CD71, CD40L (CD154) and 4-1BB (CD137); lack intracellular expression of a cytokine selected from the group consisting of IL-2, IFN-gamma and TNF-alpha; are in the G0 or G₀G₁ₐ stage of the cell cycle; and/or contain an active SAMHD1. In some instances, the resting T cells are surface negative for CD25 and CD69 (CD25-/CD69-). In some aspects, the resting T cells contain CD4+ and/or CD8+ T cells.

In some of any such embodiments, the target antigen is associated with a disease or disorder. In some cases, the disease or disorder is an infectious disease or condition, an autoimmune disease, an inflammatory disease or a cancer.

In some of any such embodiments, the target antigen is selected from the group consisting of ROR1, Her2, L1-CAM, CD19, CD20, CD22, mesothelin, CEA, hepatitis B surface antigen, anti-folate receptor, CD23, CD24, CD30, CD33, CD38, CD44, EGFR, EGP-2, EGP-4, EPHa2, ErbB2, ErbB3, ErbB4, FBP, fetal acetylcholine receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGE A3, CE7, Wilms Tumor 1 (WT-1) and cyclin A1 (CCNA1). In some embodiments of the compositions described herein, the primary human T cells are genetically engineered to express a CAR which contains an extracellular antigen-recognition domain that specifically binds to a target antigen and an intracellular signaling domain containing an ITAM. In some instance, the intracellular signaling domain contains an intracellular domain of a CD3-zeta (CD3L) chain. In some cases, the CAR further contains a transmembrane domain linking the extracellular domain and the intracellular signaling domain. In some aspects, the transmembrane domain contains a transmembrane portion of CD28.

In some of any such embodiments, the intracellular signaling domain of the CAR further contains an intracellular signaling domain of a T cell costimulatory molecule. In some cases, the T cell costimulatory molecule is selected from the group consisting of CD28 and 41BB.

In some of any such embodiments, the composition contains a pharmaceutically acceptable carrier.

### BRIEF DESCRIPTION OF THE DRAWINGS

FIGS. 1A-1B show dot plots for side scatter (SSC; y-axis) and EGFRt surface marker expression (x axis), an indicator of transgene expression, of T cells with or without activation prior to transduction with lentiviral vector particles expressing the transgene. FIG. 1A shows dot plots for EGFRt expression after transduction with lentiviral vector particles in CD4+ and CD8+ T cells activated with anti-CD3/anti-CD28 bead reagent 1 or anti-CD3/anti-CD28 bead reagent 2 prior to transduction. FIG. 1B shows the dot plots for EGFRt expression after transduction with lentiviral vector particles at various concentrations of virus (two-fold serial dilutions from an initial concentration) in CD4+ and CD8+ T cells that were not activated prior to transduction. Percentage of cells that were EGFRt+ are also shown in the box.

FIG. 2 depicts the frequency of surrogate marker surface expression (indicative of transduction frequency) for CD4+ and CD8+ T cells at indicated days post-selection, following transduction and processing under various conditions.

### DETAILED DESCRIPTION

### I. Overview

Provided are methods of transfer of viral vectors into cells (e.g. T cells) that involve transduction of cells, such as immune cells, e.g. T cells, without prior activation of the cells and/or within a time period that is initiated no more than 24 hours after obtaining the cells from a subject and/or in which the cells have not been subjected to a temperature greater than 15° C. to 25° C., such as greater than or greater than about for more than 37°±2.0° C., for more than a few hours (and no more than 24 hours) after obtaining the cells from a subject and prior to the transduction. In some embodiments, the provided methods involve incubating and/or contacting a retroviral vector particle, such as a lentiviral vector, with a population of cells, such as immune cells, e.g. T cells, with a retroviral vector particle, such as lentiviral particles, without first, i.e. prior to the transduction, activating and/or stimulating the T cells with an ex vivo stimulation reagent (e.g. anti-CD3/anti-CD28 reagent) prior to and/or in conjunction with contacting or incubating the cells with the viral particles.

In some embodiments, the provided methods are used to genetically engineer such cells with a heterologous molecule, such as with a recombinant receptor, for example an antigen receptor, such as a chimeric antigen receptor (CAR) or transgenic T cell receptor (TCR). The resulting genetically engineered cells can be used in adoptive immunotherapy. In some such embodiments, the provided methods can be used to prepare immune cells, such as T cells, for adoptive therapy, that do not include a step of activating and/or stimulating T cells. In some aspects, by eliminating the need to activate or stimulate the cells, the provided methods shorten the process for engineering and/or preparing cells for adoptive cell therapy.

In general, retroviral-based vectors can be used to stably integrate genes of interest into cells. Although vesicular stomatitis virus G protein (VSV-g) pseudotyped lentiviral vectors can transduce non-dividing cells, poor transduction has been reported with resting T cells. Thus, with existing retroviral vectors it may not always be possible to effectively stably genetically engineer quiescent and/or resting cells, such as non-cycling myeloid cells or resting T cells, with a retroviral vector in sufficient quantities for downstream use, e.g. for use in cell therapy. In some cases, for transduction to occur in T cells, activation of T cells by engagement of the T cell receptor (TCR) or by cytokine stimulation may be required. In some cases, one observation is low level expression of LDL receptor, the binding partner of VSV-G, occurs in resting T cells. Activation has been shown to increase LDL receptor expression in some cases, thereby enhancing the uptake of the lentiviral vectors. Typically, T cells are activated for at least one day (sometimes up to 3 days or more) prior to transduction for use in adoptive T cell therapies. For example, lentiviral transduction protocols for T cells typically require activation at least 24 h prior to transduction (Amirache et al. (2014) Blood, 123:1422-1424).

In some instances, available procedures for preparing genetically engineered T cells for adoptive immunotherapy can require the sequential ex vivo steps of selection, activation, transduction and expansion. Ex vivo stimulation or activation of immune cells, such as T cells, however, may not always be desired for preparation of cells for certain adoptive immunotherapy methods. For example, the inclusion of activation and/or stimulation step(s), e.g., prior to transduction, can increase the time, cost, reagents, and/or user handling in preparing cells for adoptive cell therapy. Such outcomes can increase risk of variability among different processes and/or with cells from different subjects. Thus, in some embodiments, the provided methods are advantageous by way of eliminating an activation and/or stimulation step prior to exposure to a retroviral vector particle as compared with other methods.

Additionally, in some contexts, T cell persistence and/or exhaustion following adoptive T cell therapy can be related to the stimulation and/or activation of T cells prior to administration, e.g., prior to or during genetic engineering (e.g., introduction of nucleic acid encoding a genetically engineered molecule, such as a receptor, such as an antigen receptor, e.g. a CAR). For example, activation of T cells to facilitate transduction can result in a change in differentiation or activation state of T cells that may result and/or lead to reduced persistence in vivo when genetically engineered cells are administered to a subject. Among changes in differentiation state that may occur include, in some cases, loss of a naive phenotype, loss of memory T cell phenotypes, and/or the generation of effector cells with an exhausted T cell phenotype. Exhaustion of T cells may lead to a progressive loss of T cell functions and/or in depletion of the cells (Yi et al. (2010) Immunology, 129:474-481).

The provided methods are based on observations that sufficient transduction of primary cells obtained from a subject can be achieved by incubating and/or contacting a population of cells, such as a population of T cells, with retroviral vector particles, such as lentiviral vector particles, immediately after selecting and/or enriching the cells from a subject. In some embodiments, it is found that the process for transduction in accord with the provided methods does not appear to be restricted by the need to provide stimulation to the cells via engagement of the CD3 and/or CD28 proteins and/or upregulate LDL-receptors by first activating the cell. Without wishing to be bound by theory, it is contemplated herein that upstream processing of the selected and/or enriched cells, including apheresis collection as well as the processes in the T cell selection and/or enrichment, may already upregulate LDL-receptor expression and/or otherwise render the T cells susceptible to viral entry. Accordingly, it is found that it is possible to transduce T cells immediately post-selection without the need to activate the cell for at least 24 hours, thereby enabling a shorter process. In some cases of the provided methods, cryopreservation of enriched and/or selected cells can precede transduction so long as the enriched and/or selected cells are not subjected to an ex vivo stimulation with a stimulating agent or agents prior to the incubating and/or contacting.

In some embodiments, the provided methods involve incubating and/or contacting an input composition containing cells to be transduced, in which, prior to incubating, the cells of the input composition have not been subjected to an ex vivo stimulation including incubation with an agent that turns on or initiates TCR/CD3 intracellular signaling cascade in a T cell, such as a CD4⁺ T cell or a CD8⁺ T cell. Such agents include binding molecules or antibodies, such as those specific for a TCR component and/or costimulatory receptor, e.g., anti-CD3, anti-CD28, for example, bound to solid support such as a bead, and/or one or more cytokines, e.g. recombinant cytokines, such as IL-2 and/or IL-15 and/or IL-7. In some embodiments, prior to introducing a provided retroviral vector, the provided methods do not include activating and/or stimulating a population of cells containing T cells with one or more of an anti-CD3 antibody, anti-CD28 antibody and/or recombinant IL-2, IL-15 or IL-7 cytokines. In some embodiments, reference to recombinant refers to cytokines normally not obtained in a sample obtained from the subject, but instead those that are produced using recombinant DNA technology, such as involving expression in a cell from DNA into which the gene sequence that codes for the protein has been artificially or exogenously introduced into the cell. Hence, reference to a recombinant cytokines does not include cytokines that may be present in a sample from a subject, e.g. apheresis sample, and/or present in serum obtained from such a subject. In some embodiments, the input composition comprises a population of primary cells that have been obtained from a sample from a subject and/or enriched for a particular subset of cells, e.g. T cells.

In some embodiments, the population of cells, e.g. input composition, can be a population of cells that has previously been subject to cryopreservation. In some such embodiments, prior to the cryopreservation of the cells, the cell population has not been subjected to and/or exposed and/or incubated in the presence of any condition that promotes cell activation or stimulation, such as cell activation or stimulation of T cells. In some embodiments, prior to the cryopreservation of the cells, the cell population has not been subjected to and/or exposed and/or incubated in the presence of one or more agents capable of activating an intracellular signaling domain of a TCR complex, such as an anti-CD3 and/or anti-CD28 antibody and/or one or more cytokines, such as IL-2 and/or IL-15 and/or IL-7.

In some embodiments, the population of cells, e.g. input composition, that is incubated with a provided retroviral vector is one that has not been exposed and/or subjected to a temperature, prior to incubation with the retroviral vector particle, that is greater than 0° C., such as greater than 4° C., 15° C., 20° C. or greater than 25° C. for more than about 1 hour, 3 hours, 6 hours, 12 hours, 24 hours, 36 hours, 48 hours, or 72 hours.

In some embodiments, the incubating and/or contacting is initiated no more than or no more than about 1 hour, 3 hours, 6 hours, 12 hours, 18 hours or 24 hours after obtaining a sample, e.g. apheresis sample, from the subject containing the primary cells. In some embodiments, prior to the incubating, the T cells have not been subjected to a temperature greater than or greater than about 15° C., 18° C., 22° C. or 25° C. for a duration of more than 1 hour, 2 hours, 4 hours, 6 hours, 8 hours, 12 hours, or 24 hours after obtaining the sample from the subject, such as have not been subjected to a temperature greater than or greater than about 37°±2.0° C. for a duration of more than 15 minutes, 30 minutes, 1 hour or 2 hours after obtaining the sample from the subject.

In some embodiments, the provided methods produce an output composition containing transduced cells in which such activation of the cells did not precede transduction. In some embodiments, the methods can be used to transduce a population of T cells in which at least 40%, 50%, 60%, 70%, 80%, 90% or more of the T cells in the population are resting T cells, such as T cells that lack a T cell activation marker, such as a surface marker or intracellular cytokine or other marker, and/or T cells that are in the G₀ or G₀G₁ₐ stage of the cell cycle.

In some embodiments, the methods produce an output composition in which at least 25%, at least 30%, at least 40%, at least 50%, or at least 75% of the total cells (or of a particular target cell type, such as T cells) in the output composition, are transduced with said viral vector and/or express the recombinant gene product encoded thereby.

In some embodiments, the provided methods result in a relatively high transduction of immune cells, such as non-cycling and/or resting cells, such as non-activated or non-stimulated immune cells, for example resting T cells. In some embodiments, at least 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90% or 95% of the non-cycling or resting cells, such as resting T cells, in a cell population, e.g. output composition, are transduced with retroviral vector particles in accord with the provided methods.

In some embodiments, no more than 5%, 10%, 20%, 30%, or 40% of the T cells in the input composition and/or output composition are activated cells, express a surface marker selected from the group consisting of HLA-DR, CD25, CD69, CD71, CD40L and 4-1BB; comprise intracellular expression of a cytokine selected from the group consisting of IL-2, IFN-gamma, TNF-alpha, are in the G1 or later phase of the cell cycle and/or are capable of proliferating. For example, in some aspects, the population of cells of the input composition and/or output composition is one in which at least 40%, 50%, 55%, 60%, 65%, 70%, 75%, 80%, 85%, 90%, 95% are surface negative for CD25 and/or CD69, e.g. CD25- and CD69-.

Methods and techniques for evaluating and/or assessing the cycling status of a cell, such as a resting T cell, are known (Tumeh et al. (2010) J Immunother., 33:759-768). In some embodiments, resting T cells exhibit a smaller size than proliferating T cells, such as activated T cells. Hence, in some aspects, cell size, such as determined using an automated cell counter with or without a viability dye such as trypan blue, can be employed. In some embodiments, a majority of the cells and/or substantially all of the T cells in the population are cells that have a diameter that is less than 4 μm, such as generally less than 3 μm, for example from or from about 1 μm to 3 μm, such as generally about or approximately 2 μm. In some embodiments, uptake of a metabolic substrate can be assessed, for example tritium (³H)-labeled 2′-deoxy-D-glucose ([³H]-2′DDG), ³H-labeled 3′-deoxy-3′-fluorothymidine ([³H]-3′FLT) and/or ³H-labeled-2′-deoxy-2′-fluoroarabinofluranosylcytosine ([³H]-FAC), which are each markers that are taken up by activated or stimulated cells. In some embodiments, a majority of the cells and/or substantially all of the T cells in the population are not able to take up and/or do not accumulate a marker of a metabolic substrate. In some embodiments, cell cycle can be assessed or monitored directly, such as by quantitation of DNA content, for example, utilizing a propidium iodide (PI), 7-aminoactinomycin-D (7-AAD), Hoechst 33342, 33258 and S769121, TO-PRO-3, 4′6′-diamidino-2-phenylindole (DAPI), DRAQ5™ or DRAQ7™.

Methods and techniques for assessing the expression and/or levels of a T cell activation marker are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods.

In some embodiments, the methods are capable of achieving at least a particular transduction efficiency under certain conditions. For example, in some embodiments, where the input composition includes the virus and cells at a ratio of from or from about 1 infectious unit (IU) per one of the cells to 10 IU per one of the cells, such as at least or at or about 1 infectious units (IU) per one of the cells, or at least or at or about 2 IU per one of the cells, at least or at or about 5 IU per one of the cells, or at least or at or about 10 IU per one of the cells, the method is capable of producing an output composition in which at least 10%, at least 25%, at least 30%, at least 40%, at least 50%, or at least 75% of the cells in the composition generated by the method comprise, e.g., have been transduced with, the recombinant viral vector.

In some such embodiments, the transduction efficiency of cells with a retroviral vector particle can be monitored and/or observed by measuring the level of expression of a recombinant molecule or protein, such as a heterologous molecule or protein, encoded by a nucleic acid contained in the genome of the retroviral vector following transduction or other form of transfer of the vector into a cell, such as a non-cycling host cell, such a resting T cell, or population of cells thereof. A number of well-known methods for assessing expression level of recombinant molecules may be used, such as detection by affinity-based methods, e.g., immunoaffinity-based methods, e.g., in the context of cell surface proteins, such as by flow cytometry. In some examples, the expression is measured by detection of a transduction marker and/or reporter construct. In some embodiments, nucleic acid encoding a truncated surface protein is included within the vector and used as a marker of expression and/or enhancement thereof.

In some embodiments, the provided methods can include a cryopreservation step prior to or following the incubation, e.g. transduction, of cells with the viral particles. In some embodiments, such a step could provide a break step in the process to allow for shipment of materials, sampling of materials, or ‘hold’ on therapy pending the patient's condition.

While the provided method, in some aspects, does not include a step for prior activation and/or stimulation of the cells, the method can include activation or stimulation of cells concurrently with transduction and/or following transduction. Activation or stimulation can be carried out ex vivo or in vivo. In some embodiments, following incubation, e.g. transduction, of cells with the viral particles, cells can be infused into the patient for in vivo activation and expansion

In some embodiments, during or subsequent to the incubation, the provided methods can further include culturing the input composition, output composition and/or the transduced cells ex vivo, such as under conditions for stimulation of the cells, for example to induce their proliferation and/or activation. In some embodiments, the culturing is carried out at a temperature greater than 30° C., such as greater than or greater than about 32° C., 35° C. or 37° C., for greater than 24 hours, 2 days, 3 days, 4 days, 5 days, 6 days, 7 days, 8 days, 9 days or more. In some embodiments, the stimulation is carried out in the presence of one or more stimulating agent. In some embodiments, the one or more stimulating agent is a CD3-binding molecule, a CD28-binding molecule, or is a cytokine, such as recombinant IL-2, recombinant IL-15 or recombinant IL-7. In some embodiments, the binding molecule is an antibody or antigen-binding fragment, such as an anti-CD3 antibody and/or an anti-CD28 antibody. In some embodiments, the further culturing is carried out under conditions to effect expansion of the cells, such as to yield a therapeutically effective dose of cells for administration to a subject by adoptive cell therapy.

In some embodiments, the output composition or the transduced cells are not further cultured ex vivo at a temperature greater than 30° C. and/or are not further cultured in the presence of one or more stimulating agent.

In some embodiments, the provided methods avoid substantially changing and/or minimizing changes to the differentiation state of T cells ex vivo in the process of introducing, transferring and/or transducing T cells with a nucleic acid encoding a recombinant receptor, such as a CAR. In some embodiments, the administered cells produced in accord with the provided methods exhibit a less exhausted phenotype.

In some embodiments, the input composition and/or output composition, e.g. comprising T cells, includes a lower proportion or percentage of effector cells, such as effector cells with an exhausted T cell phenotype, than cells in a composition in which an cells of an input composition had been activated and/or stimulated prior to the incubation, such as activated and/or stimulated with an anti-CD3/anti-CD28 antibody for at least or about at least 24 hours. In some embodiments, T cells in the input composition and/or output composition contain a lower proportion or percentage T effector memory (TEM) or T effector cells (TEFF) cells, such as CD45RO⁺CD62L-CCR7⁻ cells than a composition in which an cells of an input composition had been activated and/or stimulated prior to the incubation, such as activated and/or stimulated with an anti-CD3/anti-CD28 antibody for at least or about at least 24 hours. In some embodiments, the proportion or percentage is at least or about at least 1.5-fold, 2-fold, 3-fold, 4-fold or 5-fold lower.

In some embodiments, the input composition and/or output composition, e.g. comprising T cells, exhibits a higher proportion or percentage of T cells with a memory T cell phenotype than cells in a composition in which cells of an input composition had been activated and/or stimulated prior to the incubation, such as activated and/or stimulated with an anti-CD3/anti-CD28 antibody for at least or about at least 24 hours. For example, in some embodiments, the memory T cells are cells having a T central cell memory (TCM) phenotype, e.g. CD45RO⁺CCR7⁺CD62L⁺ T cells and/or CD45RO⁺CCR7⁺CD27⁺CD28⁺CD62L⁺ T cells. In some embodiments, the proportion or percentage is at least or at least about 1.5-fold, 2-fold, 3-fold, 4-fold or 5-fold lower.

In some embodiments, the provided methods are carried out such that one, more, or all steps in the preparation of cells for clinical use, e.g., in adoptive cell therapy, are carried out without exposing the cells to non-sterile conditions and without the need to use a sterile room or cabinet. In some embodiments of such a process, the cells are isolated, separated or selected, transduced, washed, optionally activated or stimulated and formulated, all within a closed system. In some embodiments, the closed system is or includes a device comprising a centrifugal chamber. In some embodiments, the methods are carried out in an automated fashion. In some embodiments, one or more of the steps is carried out apart from the closed system or device, such as apart from a centrifugal chamber system.

In some embodiments, the provided methods provide for an optimized or improved process where the cells are processed ex vivo for a shorter period thus saving time, allowing costs reduction. In some aspects, the provided methods also may produce transduced cells for administration to a subject with a better or more desirable phenotype, e.g. less exhausted cells and/or greater central memory over effector memory cells.

In some embodiments, such cells produced by the method, or a composition comprising such cells, are administered to a subject for treating a disease or condition.

In some embodiments, the provided methods include administering to a subject a sub-optimal dose of cells. In some embodiments, the dose of cells is less than or less than about 1.5-fold, 2-fold, 3-fold, 4-fold, 5-fold or 10-fold less than a therapeutically effective dose of cells for treating the disease or condition. In such an example, expansion of cells to yield a therapeutically effective amount of cells can occur in vivo upon administration of cells to a subject.

In some embodiments, the provided methods include in vivo expansion of cells. In some aspects, in vivo expansion of cells can occur in vivo by transgene-specific activation or stimulation of the administered cells. In some embodiment, the antigen receptor (e.g. CAR) is stimulated, such as is activated or expanded, upon recognition of antigen. In some embodiments, one or more agent is administered to the subject to boost, augment or increase the stimulation, activation or expansion of the cells in vivo in the subject.

In some embodiments, the provided methods produce genetically engineered T cells that, when administered to a subject, exhibit increased persistence and/or reduced T cells exhaustion. In some embodiments, a genetically engineered cell with increased persistence and/or reduced exhaustion exhibits better potency in a subject to which it is administered. In some embodiments, the provided retroviral vector particles and methods reduce variability in treatment outcomes in adoptive immunotherapy methods, for example, by minimizing and/or reducing the ex vivo manipulations of T cells prior to administration to a subject. In some embodiments, eliminating the ex vivo activation of T-cells improves the process of producing or preparing genetically engineered T-cells for adoptive immunotherapy by reducing time and reagents required for ex vivo manipulations.

In some embodiments, selection for the transduced or engineered cells is carried out following genetic engineering. In some embodiments, engineered non-cycling and/or resting or quiescent cells, such as resting T cells, can be enriched for use in adoptive immunotherapy.

Also among the provided embodiments are the resulting genetically engineered cells, such as cells expressing a genetically engineered receptor, such as a recombinant antigen receptor such as CARs and/or other recombinant receptor, and methods and uses of such genetically engineered cells for adoptive immunotherapy.

### II. Methods of Transduction

Provided herein is a method of incubating or contacting cells of an input composition with a retroviral vector particle, e.g. lentiviral vector particles. In some aspects, the input composition is a composition of primary cells obtained from a subject, in which, in some cases, a subpopulation or subset of cells has been selected and/or enriched. Features of the input composition are provided.

In some embodiments, the cells include one or more nucleic acids introduced via genetic engineering in accord with the provided methods, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

The processing steps of the methods may include any one or more of a number of cell processing steps, alone or in combination. In particular embodiments, the processing steps include transduction of the cells with viral vector particles containing a retroviral vector, such as one encoding a recombinant product for expression in the cells. The methods may further and/or alternatively include other processing steps, such as steps for the isolation, separation, selection, washing, suspension, dilution, concentration, and/or formulation of the cells. In some cases, the methods also can include an ex vivo step for cultivation (e.g., stimulation of the cells, for example, to induce their proliferation and/or activation). In other cases, a step for stimulating or activating cells is carried out in vivo upon administration of cells to a subject, by recognition of antigen and/or following administration of one or more agents to boost or augment expansion, activation and/or proliferation of cells in the subject. In some embodiments, the methods include isolating cells from the subject, preparing, processing, culturing, and/or engineering them, and re-introducing them into the same subject, before or after cryopreservation.

In some embodiments, the method includes processing steps carried out in an order in which: cells, e.g. primary cells, are first isolated, such as selected or separated, from a biological sample; selected cells are incubated with viral vector particles for transduction; and transduced cells are formulated in a composition. In some cases, transduced cells are activated, expanded or propagated ex vivo, such as by stimulation in the presence of a stimulation reagent. In some embodiments, the method can include one or more processing steps from among washing, suspending, diluting and/or concentrating cells, which can occur prior to, during or simultaneous with or subsequent to one or more of the isolation, such as separation or selection, transduction, stimulation, and/or formulation steps.

In some embodiments, one or more or all of the processing steps, e.g., isolation, selection and/or enrichment, processing, incubation in connection with transduction and engineering, and formulation steps is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps. In one example, the system is a system as described in International Patent Application, Publication Number WO2009/072003, or US 20110003380 A1. In one example, the system is a system as described in International Publication Number WO2016/073602.

In some embodiments, one or more of the cell processing steps in connection with preparing, processing and/or incubating cells in connection with the provided transduction method can be carried out in the internal cavity of a centrifugal chamber, such as a substantially rigid chamber that is generally cylindrical in shape and rotatable around an axis of rotation, which can provide certain advantages compared to other available methods. In some embodiments, all processing steps are carried out in the same centrifugal chamber. In some embodiments, one or more processing steps are carried out in different centrifugal chambers, such as multiple centrifugal chambers of the same type. Such methods include any of those as described in International Publication Number WO2016/073602.

Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Depending on the particular process (e.g. dilution, wash, transduction, formulation), it is within the level of a skilled artisan to choose a particular kit that is appropriate for the process. Exemplary kits for use with such systems include, but are not limited to, single-use kits sold by BioSafe SA under product names CS-430.1, CS-490.1, CS-600.1 or CS-900.2.

In some embodiments, the system is included with and/or placed into association with other instrumentation, including instrumentation to operate, automate, control and/or monitor aspects of the various processing steps performed in the system. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the system, such as a closed system, is sterile. In some embodiments, all connections of components of the system, such as between tubing line and a container via a connector, are made under sterile conditions. In some embodiments, connections are made under laminar flow. In some embodiments, connections are made using a sterile connection device that produces sterile connections, such as sterile welds, between a tubing and a container. In some embodiments, a sterile connection device effects connection under thermal condition high enough to maintain sterility, such as temperatures of at least 200° C., such as at least 260° C. or 300° C.

In some embodiments, the system may be disposable, such as a single-use kit. In some embodiments, a single-use kit can be utilized in a plurality of cycles of a process or processes, such as at least 2, 3, 4, 5 or more times, for example, in processes that occur in a continuous or a semi-continuous manner. In some embodiments, the system, such as a single-use kit, is employed for processing of cells from a single patient.

The centrifugal chamber generally is rotatable around an axis of rotation, and the cavity typically is coaxial with the chamber. In some embodiments, the centrifugal chamber further includes a movable member, such as a piston, which generally is capable of movement (e.g., axial movement) within the chamber, to vary the volume of the cavity. Thus, in particular embodiments, the internal cavity is bound by the side wall and end wall of the chamber and the movable member, and has a variable volume that may be adjusted by moving the movable member. The movable member may be made of rigid, substantially or generally rigid, flexible materials, or combinations thereof.

The chamber generally also includes one or more opening(s), such as one or more inlet, one or more outlet, and/or one or more inlet/outlet, which can permit intake and expression of liquid and/or gas to and from the cavity. In some cases, the opening can be an inlet/outlet where both intake and expression of the liquid and/or gas occurs. In some cases, the one or more inlets can be separate or different from the one or more outlets. The opening or openings may be in one of the end walls. In some embodiments, liquid and/or gas is taken into and/or expressed from the cavity by movement of the movable member to increase and/or decrease the cavity's volume. In other embodiments, liquid and/or gas may be taken into and/or expressed from the cavity through a tubing line or other channel that is or is placed in connection with the opening, for example, by placing the line or channel in connection with and control of a pump, syringe, or other machinery, which may be controlled in an automated fashion.

In some embodiments, the chamber is part of a closed system, such as a sterile system, having various additional components such as tubing lines and connectors and caps, within which processing steps occur. Thus, in some embodiments, the provided methods and/or steps thereof are carried out in a completely closed or semi-closed environment, such as a closed or semi-closed sterile system, facilitating the production of cells for therapeutic administration to subjects without the need for a separate sterile environment, such as a biosafety cabinet or room. The methods in some embodiments are carried out in an automated or partially automated fashion.

In some embodiments, the chamber is associated with a centrifuge, which is capable of effecting rotation of the chamber, such as around its axis of rotation. Rotation may occur before, during, and/or after the incubation in one or more of the processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In aspects of the methods, the processes need not be performed in the same closed system, such as in the same centrifugal chamber, but can be performed under a different closed system, such as in a different centrifugal chamber; in some embodiments, such different centrifugal chambers are at the respective points in the methods placed in association with the same system, such as placed in association with the same centrifuge. In some embodiments, all processing steps are performed in a closed system, in which all or a portion of each one or more processing step is performed in the same or a different centrifugal chamber.

A. Samples and Cell Preparations

The cells generally are eukaryotic cells, such as mammalian cells, and typically are human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs).

The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells are derived from cell lines, e.g., T cell lines. The cells in some embodiments are obtained from a xenogeneic source, for example, from mouse, rat, non-human primate, and pig.

In some embodiments, the cells may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, prior to the enriching and/or selecting of cells, the sample is contacted with and/or contains serum or plasma, such as human serum or plasma. In some embodiments, the serum or plasma is autologous to the subject from which the cells were obtained. In some embodiments, the serum or plasma is present in the sample at a concentration of at least or at least about 10% (v/v), at least or at least about 15% (v/v), at least or at least about 20% (v/v), at least or at least about 25% (v/v), at least or at least about 30% (v/v), at least or at least about 35% (v/v), or at least or at least about 40% (v/v). In some embodiments, prior to the selection and/or transduction of cells, the sample containing primary cells is contacted with or contains an anticoagulant. In some embodiments, the anti-coagulant is or contains free citrate ion, e.g. anticoagulant citrate dextrose solution, Solution A (ACD-A).

In some embodiments, the prior to the enriching and/or selecting cells, cells from a sample are transferred or suspended in a serum-free media. In some embodiments, the serum free media is a defined and/or well-defined cell culture media. In certain embodiments, the serum free media is a controlled culture media that has been processed, e.g., filtered to remove inhibitors and/or growth factors. In some embodiments, the serum free media contains proteins. In certain embodiments, the serum-free media may contain serum albumin, hydrolysates, growth factors, hormones, carrier proteins, and/or attachment factors. In some embodiments, the serum-free media contains proteins, e.g., albumin, such as bovine serum albumin, human serum albumin, and/or recombinant albumin. In some embodiments, the serum free media contains a basal media, e.g., DMEM or RPMI 1640, containing amino acids, vitamins, inorganic salts, buffers, antioxidants and energy sources. In some embodiments, the serum free media is supplemented, such as with, but not limited to, albumin, chemically defined lipids, growth factors, insulin, cytokines, and/or antioxidants. In some embodiments, the serum free media is formulated to support growth, proliferation, health, homeostasis of cells of a certain cell type, such as immune cells, T cells, and/or CD4+ and CD8+ T cells.

In some embodiments, prior to the selection and/or enrichment of cells, the sample or the cells in the sample can be rested or held prior to further processing steps. In some embodiments, the sample is maintained at or held at a temperature of from or from about 2° C. to 8° C. for up to 48 hours, such as for up to 12 hours, 24 hours or 36 hours.

In some embodiments, the preparation methods include steps for freezing, e.g., cryopreserving, the cells, either before or after isolation, selection and/or enrichment and/or incubation for transduction and engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 10 per minute and stored in the vapor phase of a liquid nitrogen storage tank.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8⁺ T cells and CD4⁺ T cells further enhances efficacy.

In embodiments, memory T cells are present in both CD62L⁺ and CD62L-subsets of CD8⁺ peripheral blood lymphocytes. PBMC can be enriched for or depleted of CD62L-CD8⁺ and/or CD62L+CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or subpopulation, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naïve CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L- and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1. In one example, the system is a system as described in International Publication Number WO2016/073602.

In some embodiments, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber.

For example, immunoaffinity-based selection can depend upon a favorable energetic interaction between the cells being separated and the molecule specifically binding to the marker on the cell, e.g., the antibody or other binding partner on the solid surface, e.g., particle. In certain available methods for affinity-based separation using particles such as beads, particles and cells are incubated in a container, such as a tube or bag, while shaking or mixing, with a constant cell density-to-particle (e.g., bead) ratio to aid in promoting energetically favored interactions. Such approaches may not be ideal for use with large-scale production, for example, in that they may require use of large volumes in order to maintain an optimal or desired cell-to-particle ratio while maintaining the desired number of cells. Accordingly, such approaches can require processing in batch mode or format, which can require increased time, number of steps, and handling, increasing cost and risk of user error.

In some embodiments, by conducting such selection steps or portions thereof (e.g., incubation with antibody-coated particles, e.g., magnetic beads) in the cavity of the centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g. bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

The incubation with a selection reagent or reagents, e.g., as part of selection methods which may be performed in the chamber cavity, include using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g. is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least or about or 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from or from about 5 minutes to 6 hours, such as 30 minutes to 3 hours, for example, at least or about at least 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the further selection is performed outside of the centrifugal chamber. In some embodiments, the separation is performed in the same closed system in which the centrifugal chamber is present and in which the incubation of cells with the selection reagent was performed. In some embodiments, after incubation with the selection reagents, incubated cells, including cells in which the selection reagent has bound, are expressed from the centrifugal chamber, such as transferred out of the centrifugal chamber, into a system for immunoaffinity-based separation of the cells. In some embodiments, the system for immunoaffinity-based separation is or contains a magnetic separation column. In some embodiments, prior to separation, one or more other processing steps can be performed in the chamber, such as washing.

In some aspects, the separation and/or other steps is carried out using CliniMACS system (Miltenyi Biotic), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, cells for transduction by the provided retroviral vector particles include, for example, monocytes, monocyte-derived macrophages, monocyte-derived dendritic cells or resting T cells. In particular embodiments, the provided retroviral particles can transduce resting T cells. In some embodiments, the input composition comprises a plurality of cells, such as immune cells, e.g. T cells, that are non-cycling and/or quiescent and/or resting and/or in which a majority of cells, e.g. greater than 50%, 60%, 70%, 80%, 80% or more cells, in a population so transduced are non-cycling and/or quiescent and/or resting. In some embodiments, the input composition comprises a population of T cells in which at least 40%, 50%, 60%, 70%, 80%, 90% or more of the T cells in the population are resting T cells, such as T cells that lack a T cell activation marker, such as a surface marker or intracellular cytokine or other marker, and/or T cells that are in the G₀ or G₀G₁ₐ stage of the cell cycle. In some embodiments, the cells contain an active SAMHD1, such as an unphosphorylated SAMHD1. In some embodiments, the cells are in the G₀, G₀/G₁ₐ or G1 stage of the cell cycle.

In some embodiments, the methods involve transduction of T cells in which no more than 5%, 10%, 20%, 30%, or 40% of the T cells express a T cell activation marker. In some embodiments, no more than 5%, 10%, 20%, 30%, or 40% of the T cells are surface positive for one or more T cell activation markers HLA-DR, CD25, CD69, CD71, CD40L (CD154) and/or 4-1BB (CD137). In some embodiments, no more than 5%, 10%, 20%, 30%, or 40% of the T cells have intracellular expression of a cytokine that is IL-2, IFN-gamma and/or TNF-alpha.

B. Viral Vector Particles

In some embodiments, the viral vector particles are retroviral vector particles, such as lentiviral particles, containing a nucleic acid encoding a recombinant and/or heterologous molecule, e.g., recombinant or heterologous protein, such as a recombinant and/or heterologous receptor, such as chimeric antigen receptor (CAR) or other antigen receptor, in a genome of the viral vector. The genome of the viral vector particle typically includes sequences in addition to the nucleic acid encoding the recombinant molecule. Such sequences may include sequences that allow the genome to be packaged into the virus particle and/or sequences that promote expression of a nucleic acid encoding a recombinant receptor, such as a CAR.

I. Viral Vector

In some embodiments, the viral vector particles contain a genome derived from a retroviral genome based vector, such as derived from a lentiviral genome based vector. In some aspects of the provided viral vectors, the heterologous nucleic acid encoding a recombinant receptor, such as an antigen receptor, such as a CAR, is contained and/or located between the 5′ LTR and 3′ LTR sequences of the vector genome.

In some embodiments, the viral vector genome is a lentivirus genome, such as an HIV-1 genome or an SIV genome. For example, lentiviral vectors have been generated by multiply attenuating virulence genes, for example, the genes env, vif, vpu and nef can be deleted, making the vector safer for therapeutic purposes. Lentiviral vectors are known. See Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

Non-limiting examples of lentiviral vectors include those derived from a lentivirus, such as Human Immunodeficiency Virus 1 (HIV-1), HIV-2, an Simian Immunodeficiency Virus (SIV), Human T-lymphotropic virus 1 (HTLV-1), HTLV-2 or equine infection anemia virus (E1AV). For example, lentiviral vectors have been generated by multiply attenuating the HIV virulence genes, for example, the genes env, vif, vpr, vpu and nef are deleted, making the vector safer for therapeutic purposes. Lentiviral vectors are known in the art, see Naldini et al., (1996 and 1998); Zufferey et al., (1997); Dull et al., 1998, U.S. Pat. Nos. 6,013,516; and 5,994,136). In some embodiments, these viral vectors are plasmid-based or virus-based, and are configured to carry the essential sequences for incorporating foreign nucleic acid, for selection, and for transfer of the nucleic acid into a host cell. Known lentiviruses can be readily obtained from depositories or collections such as the American Type Culture Collection (“ATCC”; 10801 University Blvd., Manassas, Va. 20110-2209), or isolated from known sources using commonly available techniques.

In some embodiments, the viral genome vector can contain sequences of the 5′ and 3′ LTRs of a retrovirus, such as a lentivirus. In some aspects, the viral genome construct may contain sequences from the 5′ and 3′ LTRs of a lentivirus, and in particular can contain the R and U5 sequences from the 5′ LTR of a lentivirus and an inactivated or self-inactivating 3′ LTR from a lentivirus. The LTR sequences can be LTR sequences from any lentivirus from any species. For example, they may be LTR sequences from HIV, SIV, FIV or BIV. Typically, the LTR sequences are HIV LTR sequences.

In some embodiments, the nucleic acid of a viral vector, such as an HIV viral vector, lacks additional transcriptional units. The vector genome can contain an inactivated or self-inactivating 3′ LTR (Zufferey et al. J Virol 72: 9873, 1998; Miyoshi et al., J Virol 72:8150, 1998). For example, deletion in the U3 region of the 3′ LTR of the nucleic acid used to produce the viral vector RNA can be used to generate self-inactivating (SIN) vectors. This deletion can then be transferred to the 5′ LTR of the proviral DNA during reverse transcription. A self-inactivating vector generally has a deletion of the enhancer and promoter sequences from the 3′ long terminal repeat (LTR), which is copied over into the 5′ LTR during vector integration. In some embodiments enough sequence can be eliminated, including the removal of a TATA box, to abolish the transcriptional activity of the LTR. This can prevent production of full-length vector RNA in transduced cells. In some aspects, the U3 element of the 3′ LTR contains a deletion of its enhancer sequence, the TATA box, Sp1 and NF-kappa B sites. As a result of the self-inactivating 3′ LTR, the provirus that is generated following entry and reverse transcription contains an inactivated 5′ LTR. This can improve safety by reducing the risk of mobilization of the vector genome and the influence of the LTR on nearby cellular promoters. The self-inactivating 3′ LTR can be constructed by any method known in the art. In some embodiments, this does not affect vector titers or the in vitro or in vivo properties of the vector.

Optionally, the U3 sequence from the lentiviral 5′ LTR can be replaced with a promoter sequence in the viral construct, such as a heterologous promoter sequence. This can increase the titer of virus recovered from the packaging cell line. An enhancer sequence can also be included. Any enhancer/promoter combination that increases expression of the viral RNA genome in the packaging cell line may be used. In one example, the CMV enhancer/promoter sequence is used (U.S. Pat. Nos. 5,385,839 and 5,168,062).

In certain embodiments, the risk of insertional mutagenesis can be minimized by constructing the retroviral vector genome, such as lentiviral vector genome, to be integration defective. A variety of approaches can be pursued to produce a non-integrating vector genome. In some embodiments, a mutation(s) can be engineered into the integrase enzyme component of the pol gene, such that it encodes a protein with an inactive integrase. In some embodiments, the vector genome itself can be modified to prevent integration by, for example, mutating or deleting one or both attachment sites, or making the 3′ LTR-proximal polypurine tract (PPT) non-functional through deletion or modification. In some embodiments, non-genetic approaches are available; these include pharmacological agents that inhibit one or more functions of integrase. The approaches are not mutually exclusive; that is, more than one of them can be used at a time. For example, both the integrase and attachment sites can be non-functional, or the integrase and PPT site can be non-functional, or the attachment sites and PPT site can be non-functional, or all of them can be non-functional. Such methods and viral vector genomes are known and available (see Philpott and Thrasher, Human Gene Therapy 18:483, 2007; Engelman et al. J Virol 69:2729, 1995; Brown et al J Virol 73:9011 (1999); WO 2009/076524; McWilliams et al., J Virol 77:11150, 2003; Powell and Levin J Virol 70:5288, 1996).

In some embodiments, the vector contains sequences for propagation in a host cell, such as a prokaryotic host cell. In some embodiments, the nucleic acid of the viral vector contains one or more origins of replication for propagation in a prokaryotic cell, such as a bacterial cell. In some embodiments, vectors that include a prokaryotic origin of replication also may contain a gene whose expression confers a detectable or selectable marker such as drug resistance.

2 SAMHD1-Inhibiting Accessory Protein

Among the provided embodiments are retroviral vector particles that contain a SAMHD1-inhibiting accessory protein encapsulated in their virion. In some aspects, such particles permit the efficient transduction of non-cycling cells, such as non-cycling immune cells, for example, resting T cells. In some aspects, such particles permit efficient transduction of monocytes, monocyte-derived macrophages or monocyte-derived dendritic cells. In some aspects, the provided retroviral vector particles minimize and/or reduce the requirement for ex vivo manipulations of cells for transduction, such as those for activation and/or stimulation of cells.

SAMHD1 is a protein present in some non-cycling and/or quiescent immune cells that has dGTP-dependent deoxynucleotide triphosphohydrolase activity and can dephosphorylate cellular deoxynucleotide triphosphates (dNTPs). The presence of active SAMHD1 in cells that has antiviral activity, such as unphosphorylated SAMHD1, can reduce the nucleotide pool. Such reduction can thereby restrict retroviral transduction by preventing the viral vector from encountering sufficient numbers of nucleotides to facilitate sufficient reverse transcription of viral RNA, e.g., of the viral genome. While in some cases cycling cells, such as activated or proliferating T cells, can express SAMHD1, the antiviral activity of SAMHD1 is, in some aspects, regulated by cell-cycle dependent phosphorylation of SAMHD1 (Cribier et al. (2013) Cell Reports, 3:1036-1043). For example, in cycling cells, SAMHD1 generally interacts with cell-cycle related proteins and, in some aspects, is phosphorylated by a cyclin-dependent kinase 1 (CDK1). An exemplary sequence of a human SAMHD1 is set forth in SEQ ID NO:19 (see e.g., UniProt No. Q9Y3Z3). For example, in some embodiments, SAMHD1 can be phosphorylated at a position corresponding to position T592 of the exemplary sequence of SAMHD1 set forth in SEQ ID NO: 19. Phosphorylated SAMHD1 has reduced and/or minimal activity, thereby accounting for the lack of viral restriction to transduction in cycling T cells.

By incorporating a SAMHD1-inhibiting accessory protein into the virion, in some embodiments of the provided vector particles and methods, SAMHD1 is inhibited, such as degraded, in cells introduced with the provided retroviral vector particle. This increases the nucleotide pool, and thereby allows transduction to occur in quiescent and/or non-cycling cells that express SAMHD1. In some embodiments, the provided retroviral particles contain a SAMHD1-inhibiting protein and can transduce cells containing an active SAMHD1, such as an unphosphorylated SAMHD1 protein. In some embodiments, transduction by provided retroviral vector particles occurs in cells in which SAMHD1 is not phosphorylated at a position corresponding to position T592 in the exemplary sequence of SAMHD1 set forth in SEQ ID NO: 19. Methods of assessing expression and/or phosphorylation of SAMHD1 can be assessed using known techniques, for example, by Western Blot using a SAMHD1-specific antibody of following SDS-PAGE and/or phosphate affinity SDS-PAGE using Phos-tag acrylamide technology or other similar methods (see e.g. Cribier et al.).

In particular embodiments, the provided retroviral vector particles modified to contain a SAMHD1-inhibiting accessory protein in their virion are any viral vector that relies on host nucleotides for transduction. Such viral vector particles can include retroviral vector particles, for example, lentiviral or gammaretroviral vector particles, which utilize cellular dNTPs to synthesize viral cDNA from viral RNA by a viral reverse transcriptase.

In some embodiments, the SAMHD1-inhibiting protein is a viral host cell protein that exhibits SAMHD1-inhibiting activity. In some embodiments, the SAMHD1-inhibiting protein is a lentiviral protein, such as a lentiviral Vpx or Vpr protein, or a variant or portion thereof that exhibits SAMHD1-inhibiting activity. Vpx and Vpr, which are expressed in different lentiviruses, share sequence and structural homology and are virion-associated proteins that are packaged into the virion as it assembles. Upon introduction to a cell, Vpx and, in some aspects Vpr, can interfere with ubiquitination of SAMHD1 by a ubiquitin ligase complex in the cell formed from CUL4A-DDB1-DCAF1 host cell proteins, thereby targeting SAMHD1 for proteosomal degradation (Schwefel et al. (2014) Nature 505:234-238). In some embodiments, inhibition, such as degradation, of SAMHD1 by a Vpr or Vpx protein is mediated by binding of the Vpx or Vpr lentiviral protein to SAMHD1 and/or to one or more of DDB 1 or DCAF1 in the ubiquitin ligase complex.

In some embodiments, the SAMHD1-inhibiting protein is an antibody or fragment thereof that specifically binds and/or inhibits SAMHD1.

In some embodiments, the provided retroviral vector particles exhibit activity to degrade SAMHD1 in a host cell, such as resting T cells, into which the viral vector particle has been introduced. In some aspects, SAMHD1 can be degraded 2-fold, 3-fold, 4-fold, 5-fold, 6-fold, 7-fold, 8-fold, 9-fold, 10-fold, 15-fold, 20-fold, 30-fold, 35-fold, 40-fold, 45-fold, 50-fold, 60-fold, 70-fold, 80-fold, 90-fold, 100-fold, 150-fold, 200-fold or more compared to the expression or level of SAMHD1 in the same cells not introduced with the retroviral vector particles. In some such embodiments, degradation of SAMHD1 can be monitored or observed following transduction or other form of transfer of the provided retroviral vector particle containing a SAMHD1-inhibiting protein into a non-cycling host cell, such as a resting T cell. Degradation can be measured by determining a relative degree of SAMHD1 expression as compared to the expression of SAMHD1 in the same cells that were not introduced with the retroviral vector particles. A number of well-known methods for assessing expression level of a protein may be used. In some embodiments, SAMHD1 can be detected by immunoblot utilizing an SAMHD1-specific antibody, for example, in a sample from lysed cells. The extent or level of expression also can be quantified and/or quantitated by standard procedures, such as by densitometry. In some embodiments, SAMHD1 levels and/or expression can be monitored over time and/or can be monitored by varying the viral input.

In some aspects, the provided retroviral vector particles containing a SAMHD1-inhibiting protein, such as a Vpx or Vpr protein, render non-cycling and/or quiescent or resting immune cells more susceptible to transduction and introduction of a genetically engineered molecule encoded by such vector, such as those encoding genetically engineered receptors, such as recombinant antigen receptors such as CARs and/or other recombinant receptors such as those containing an extracellular ligand-binding or recognition domain and an intracellular signaling domain or domains capable of potentiating a stimulatory signal to a T cell, such as ITAM-containing and/or T cell costimulatory domains. In some embodiments, provided are methods of using provided retroviral vector particles containing a SAMHD1-inhibiting protein, such as a Vpx protein or Vpr protein, to transduce and/or introduce a genetically engineered receptor, such as recombinant antigen receptors such as CARs and/or other recombinant receptors, into monocyte-derived macrophages, monocyte-derived dendritic cells or resting T cells.

In some embodiments, the SAMHD1 inhibitor is a Vpx protein, a Vpr protein or is a functional variant or portion of a Vpx or Vpr protein that exhibits SAMHD1-inhibiting activity. In some embodiments, Vpx is a 112-amino-acid (aa), 18-kDa protein found in primate lentiviruses. Vpx from SIV and HIV-2 are 83% identical at the amino acid level (Goujon et al., J Virol, 82:12335-12345 (2008), and exhibit activity to bind and degrade SAMHD1 (Laguette et al., Nature, 474: 654-657 (2011). Vpx is only present in some lentiviruses; it is not found in HIV-1. The closely related gene Vpr, which shares a high degree of structural and sequence homology to Vpx, is encoded by all primate lentiviruses, and, in some cases, has evolved to also exhibit SAMHD1 activity. Both Vpx and Vpr are packaged in the virion through its interaction with the p6 region of the p55gag precursor.

In provided particles, compositions and methods, any suitable Vpx and/or Vpr protein can be used that exhibits SAMHD1-inhibiting activity. A protein exhibits SAMHD1-inhibiting activity if it is able to inhibit, such as degrade, SAMHD1 in a cell. In some cases, inhibition can be monitored by assessing SAMHD1 degradation in a non-cycling cell, such as resting T cell, following introduction of a viral vector particle containing such a protein, compared to with a viral vector not containing such a protein, such as using methods known in the art or as described. In some embodiments, Vpx, Vpr and variants thereof are tested for the ability to inhibit the activity of SAMHD1 according to one or more of various known methods. See Lim et al., Cell Host & Microbe, 11, 194-204 (2012) and Lahouassa et al., Nature Immunol, 13:3, 223-229 (2012). In some cases, inhibition can be monitored by assessing transduction efficiency in a non-cycling cell, such as a resting T cell, following introduction of a viral vector particle containing such a protein compared to a viral vector that is comparable or is the same but that does not contain a Vpx or Vpr protein, such as using methods known in the art or as described.

In some embodiments, the Vpx and/or Vpr or variant or portion is provided for packaging and incorporation into a virion. In some embodiments, a gene encoding a Vpx protein, Vpr protein or variant or portion of a Vpx or Vpr protein is included on the lentiviral genome and is expressed when the viral particle infects a target cell.

In some embodiments, the SAMHD1-inhibiting protein is a Vpx protein that is encoded by a wild-type lentivirus, such as a wild-type primate lentivirus, or is a variant or portion thereof that exhibits SAMHD1-inhibiting activity (see e.g. Fregoso (2013) PLoS Pathogens, 9:e1003496). Among primate lentiviruses that encode Vpx are HIV-2/SIVsmc-related and SIVrcm/SIVmnd2-related lineage viruses (Lim et al. (2012) Cell Host & Microbe, 11:194-204), such as HIV-2, SIV sooty mangabey (SIVsm), SIV red capped mangabey (SIVrcm), and SIV macaque (SIVmac), SIV mandrill (SIVmnd2), SIV drill (SIVdrl), SIV pig-tailed macaque (SIVmne).

In some embodiments, the retroviral vector particles, such as lentiviral vector particles, contain a wild-type Vpx protein encoded by HIV-2, SIVsm, SIVrcm, SIVmac, SIVmnd2, SIVdrl, SIVmne, or is a variant or portion thereof that exhibits SAMHD1-inhibiting activity. In some embodiments, the Vpx protein has an amino acid sequence that is at least 75%, 80%, 85%, 90%, 95%, 96%, 97%, 98%, or 99% identical to SEQ ID NO: 1 (SIVmac Vpx) or is a functional fragment or portion of such a protein. In some embodiments, the Vpx protein has an amino acid sequence at least 75%, 80%, 85%, 90%, 95%, 96%, 97%, 98%, or 99% identical to SEQ ID NO: 2 (SIVsm Vpx) or is a functional fragment of portion of such a protein. In some embodiments, the Vpx protein has an amino acid sequence that is at least 75%, 80%, 85%, 90%, 95%, 96%, 97%, 98%, or 99% identical to SEQ ID NO: 3 (SIVrcm Vpx) or is a functional fragment or portion of such a protein. In some embodiments, the Vpx protein has an amino acid sequence that is at least 75%, 80%, 85%, 90%, 95%, 96%, 97%, 98%, or 99% identical to SEQ ID NO: 4 (HIV-2) or is a functional fragment or portion of such a protein. In some embodiments, the Vpx protein has an amino acid sequence at least 75%, 80%, 85%, 90%, 95%, 96%, 97%, 98%, or 99% identical to SEQ ID NO: 16 (SIVmnd2) or is a functional fragment or portion of such a protein. In some embodiments, the Vpx protein has an amino acid sequence that is at least 75%, 80%, 85%, 90%, 95%, 96%, 97%, 98%, or 99% identical to SEQ ID NO: 17 (SIVdrl) or is a functional fragment or portion of such a protein. In some embodiments, the Vpx protein has an amino acid sequence that is at least 75%, 80%, 85%, 90%, 95%, 96%, 97%, 98%, or 99% identical to SEQ ID NO: 18 (SIVmne) or is a functional fragment or portion of such a protein.

In some embodiments, the SAMHD1-inhibiting protein is a Vpr protein that exhibits SAMHD1-inhibiting activity. Vpr is generally encoded by natural lentiviruses. Not all Vpr proteins exhibit SAMHD1-inhibiting activity. For example, Vpr from SIVdeb, SIVmus, SIVagm, SIVden, SIVtal, SIVgsn, SIVmon and SIVsyk have been reported to exhibit SAMHD1-inhibiting activity (Lim et al. (2012) Cell Host & Microbe, 11:194-204).

In some embodiments, the SAMHD1-inhibiting protein is an SIVdeb Vpr, SIVmus Vpr, SIVagm Vpr, SIVden Vpr, SIVtal Vpr, SIVgsn Vpr, SIVmon Vpr or SIVsyk Vpr, or is a variant or portion thereof that exhibits SAMHD1-inhibiting activity. In some embodiments, the Vpr protein has an amino acid sequence that is at least 75%, 80%, 85%, 90%, 95%, 96%, 97%, 98%, or 99% identical to SEQ ID NO: 5 (SIVdeb) or is a functional fragment or portion thereof. In some embodiments, the Vpr protein has an amino acid sequence that is at least 75%, 80%, 85%, 90%, 95%, 96%, 97%, 98%, or 99% identical to SEQ ID NO: 6 (SIVmus) or is a functional fragment or portion thereof. In some embodiments, the Vpr protein has an amino acid sequence that is at least 75%, 80%, 85%, 90%, 95%, 96%, 97%, 98%, or 99% identical to SEQ ID NO: 7 (SIVagm Vpr) or is a functional fragment or portion thereof. In some embodiments, the Vpr protein has an amino acid sequence that is at least 75%, 80%, 85%, 90%, 95%, 96%, 97%, 98%, or 99% identical to SEQ ID NO: 8 (SIVagm Vpr) or is a functional fragment or portion thereof. In some embodiments, the Vpr protein has an amino acid sequence that is at least 75%, 80%, 85%, 90%, 95%, 96%, 97%, 98%, or 99% identical to SEQ ID NO: 9 (SIVdeb Vpr) or is a functional fragment or portion thereof. In some embodiments, the Vpr protein has an amino acid sequence at least 75%, 80%, 85%, 90%, 95%, 96%, 97%, 98%, or 99% identical to SEQ ID NO: 10 (SIVden Vpr) or is a functional fragment or portion thereof. In some embodiments, the variant has an amino acid sequence that is at least 75%, 80%, 85%, 90%, 95%, 96%, 97%, 98%, or 99% identical to SEQ ID NO: 11 (SIVtal Vpr) or is a functional fragment or portion thereof. In some embodiments, the Vpr protein has an amino acid sequence that is at least 75%, 80%, 85%, 90%, 95%, 96%, 97%, 98%, or 99% identical to SEQ ID NO: 12 (SIVgsn Vpr) or is a functional fragment or portion thereof. In some embodiments, the Vpr protein has an amino acid sequence at least 75%, 80%, 85%, 90%, 95%, 96%, 97%, 98%, or 99% identical to SEQ ID NO: 13 (SIVgsn Vpr) or is a functional fragment or portion thereof. In some embodiments, the Vpr protein has an amino acid sequence that is at least 75%, 80%, 85%, 90%, 95%, 96%, 97%, 98%, or 99% identical to SEQ ID NO: 14 (SIVmon Vpr) or is a functional fragment or portion thereof. In some embodiments, the Vpr protein has an amino acid sequence that is at least 75%, 80%, 85%, 90%, 95%, 96%, 97%, 98%, or 99% identical to SEQ ID NO: 15 (SIVsyk Vpr) or is a functional fragment or portion thereof.

In some embodiments, the SAMHD1-inhibiting protein, such as a Vpx or a Vpr protein, is heterologous to the virus. By heterologous in this context means that the protein is derived from a different viral family, class or species as compared to that in which it is incorporated into. For example, in some aspects, a Vpx protein from SIVmac can be incorporated into an HIV-1 vector particle. In other aspects, a Vpx protein from any lentivirus, such as from SIVmac, can be incorporated into a gammaretrovirus, such as MLV.

In some embodiments, the SAMHD1-inhibiting protein, such as a Vpx or a Vpr protein, inhibits, such as degrades, human SAMHD1, e.g. set forth in SEQ ID NO:19 (Lim et al. (2012) Cell Host & Microbe, 11:194-204). In some embodiments, the Vpx protein that degrades human SAMHD1 is encoded by HIV-2 or SIVmac or is a variant or functional fragment or portion thereof that degrades human SAMHD1. For example, in some embodiments, the Vpx protein has an amino acid sequence that is at least 75%, 80%, 85%, 90%, 95%, 96%, 97%, 98%, or 99% identical to SEQ ID NO: 1 or SEQ ID NO:4 or is a functional fragment or portion thereof that degrades human SAMHD1. In some embodiments, the Vpr protein that degrades human SAMHD1 can be encoded by SIVdeb or SIVmus or is a variant or functional fragment or portion thereof that degrades human SAMHD1. For example, in some embodiments, the Vpr protein has an amino acid sequence that is at least 75%, 80%, 85%, 90%, 95%, 96%, 97%, 98%, or 99% identical to SEQ ID NO: 5 or SEQ ID NO:6 or is a functional fragment or portion thereof that degrades human SAMHD1.

In some embodiments, variants include proteins that contain one or more amino acid deletions, insertions and or substitutions compared to a sequence of a wild-type or reference Vpx or Vpr protein. In some cases, variants include either non-conservative or conservative substitutions. In some cases, information from sequence alignments of Vpx proteins and/or Vpr proteins can be used to generate functional variants and functional fragment variants of Vpx or Vpr. For example, one or more conservative or non-conservative substitution can be introduced at positions that differ between viruses encoding a Vpx or Vpr protein.

For example, in some embodiments, residues that share identity between and among Vpx proteins encoded by SIV and HIV-2 are not varied or changed in a variant of a Vpx protein provided herein. For example, the following mutations have been shown to reduce or abolish Vpx activity: deletion of the proline-rich C-terminal 11 residue, Ser13Ala, Lys84Ala/Lys85Ala, Thr17Ala, Thr28Ala, Gly86Ala/Cys87Ala, Ser13 Ala/Thr17Ala/Thr28Ala, His39Ala, Tyr66Ala/Tyr69Ala/Tyr71Ala, Trp49Ala/Trp53Ala/Trp56Ala, Lys68Ala/Lys77Ala, Gln76Ala, Pro9Gly, Asn12Ala, Glu15Ala, Glu16Ala, Phe80Ala, each with reference to residues set forth in SEQ ID NO: 1. See Goujon et al., Gramberg et al. (2010) Journal of Virology, 84:1387-1396., Laguette et al. (2011). Hence, in some embodiments, a variant of a Vpx protein does not contain a substitution at a residue corresponding to any of the above residues in SEQ ID NO: 1 shown to be required for activity. In some embodiments, a variant of a Vpx protein contains a conservative substitution, but not a non-conservative substitution, at a residue corresponding to any of the above residues in SEQ ID NO:1.

In some embodiments, variants of a Vpx or Vpr protein can include a mutation that is known not to affect SAMHD1-inhibiting activity (See Goujon et al., J Virol, 82:12335-12345 (2008)). For example, in some embodiments, mutations such as amino acid substitutions corresponding to one or more of Asn26Ala, Ser52Ala, and Ser63Ala/Ser65Ala mutations in SEQ ID NO: 1 do not affect Vpx function.

In some embodiments, inhibition and/or degradation of SAMHD1 requires nuclear localization of a SAMHD1-inhibiting protein, such as a Vpx protein, Vpr protein or variant or portion thereof. Vpx induces SAMHD1 ubiquitination and degradation in the nucleus, such that both SAMHD1 and Vpx must localize to the nucleus. Thus, in some embodiments, a Vpx protein, Vpr protein or variant or portion thereof contains a nuclear localization signal. In some embodiments, a Vpx protein or variant or portion thereof contains a nuclear localization signal corresponding to residues 62-80 or 65-72 with reference to SEQ ID NO:4.

In some embodiments, inhibition and/or degradation of SAMHD1 involves binding and/or interaction with DCAF. Thus, in some embodiments, a Vpx protein, Vpr protein or variant or portion thereof binds to DCAF. In some embodiments, a Vpx protein, a Vpr protein or a variant or portion thereof contains a Wx4Φx2Φx3AΦxH motif (set forth in SEQ ID NO: 26; Wei et al. (2012) Cell Microbiol., 14:1745-56). For example, in some embodiments, a Vpx protein, a Vpr protein or a variant or portion thereof contains a Wx4Φx2Φx3AΦxH motif at residues corresponding to residues 24-39 with reference to SEQ ID NO: 1 or SEQ ID NO:4. In some embodiments, a Vpx protein, a Vpr protein or a variant or portion thereof contains a Gln (Q) at a position corresponding to position 76 of SEQ ID NO: 1.

In some embodiments, a Vpx protein, a Vpr protein or a variant or portion thereof contains residues corresponding to residues of the N-terminal portion, helix 1, helix 2 and/or helix 3 of the protein (Gramberg et al. (2010) Journal of Virology, 84:1387-1396). For example, a Vpx protein, a Vpr protein or a variant or portion thereof in some embodiments contains a sequence of amino acids corresponding to residues 1-86 of the sequence of amino acids set forth in SEQ ID NO:1, or an amino-acid substituted variant thereof that exhibits at least 75%, 80%, 85%, 90%, 95%, 96%, 97%, 98%, or 99% identical to residues 1 through 86 of SEQ ID NO: 1 and exhibits SAMHD1-inhibiting activity.

In some embodiments, a Vpx or a Vpr protein, or a functional portion or variant thereof, is packaged into the virion, which permits the proteins to inhibit, such as degrade, SAMHD1 upon infection of a host cell prior to proviral integration. In some embodiments, the retroviral vector particle contains a p6 domain of a Gag protein that promotes packaging of the SAMHD1-inhibiting lentiviral protein into the retroviral vector. Vpx and Vpr are packaged through interactions with an amino acid packaging motif in the p6 region of the Gag precursor polypeptide (p6gag). In some embodiments, sufficient incorporation is achieved with the endogenous viral p6 Gag. In some embodiments, incorporation is effected and/or is increased by using a chimeric p6 Gag containing a packaging motif that promotes packaging.

In some embodiments, the packaging motif corresponds to a motif that in the N terminal region of P6 comprising amino acids corresponding to amino acid residues 17 to 23 of SEQ ID NO:21 (Accola et al. (1999) J. Virol., 73:9992-9). For example, the packaging motif in some embodiments comprises a leucine-containing motif DXAXXLL (SEQ ID NO:22).

In some embodiments, the retroviral vector particle is modified by introduction of a packaging motif that mediates and/or promotes Vpx or Vpr packaging into the virion. In some embodiments, the packaging motif is inserted into the corresponding position of the endogenous viral p6 region. In some embodiments, the packaging motif comprises a sequence of amino acids DPAVDLL (SEQ ID NO:23) or DPAVDLLKNY (SEQ ID NO:24), which correspond to amino acids 17 to 23 or amino acids 17 to 26, respectively, of SIVmac p6 set forth in SEQ ID NO:21. In some embodiments, the packaging motif is any of the packaging motifs described in U.S. published Appl. No. US2013/0183334. In some embodiments, transfer of the motif into the corresponding location of HIV-1 p6 set forth in SEQ ID NO:20 results in HIV-1 virions that package Vpx.

In some embodiments, the Vpx or Vpr protein, or variant or portion thereof, is packaged without modification of the p6 region of the Gag protein. In some such embodiments, the retroviral vector is derived from HIV-1, and sufficient incorporation is achieved using the HIV-1 p6 Gag. In some such embodiments, the SAMHD1-inhibiting protein is an SIVmac Vpx or is a variant or portion thereof that exhibits SAMHD1-inhibiting activity.

In some embodiments, the retroviral vector is derived from HIV-1 and comprises a chimeric or hybrid p6 domain comprising an HIV-1 p⁶ into which is inserted an SIVmac p6 packaging motif. For example, in some embodiments, the retroviral vector comprises a chimeric or hybrid p6 domain comprising, in consecutive order, amino acid residues corresponding to amino acids 1-14 of HIV-P6 set forth in SEQ ID NO:20, amino acids corresponding to an SIVmac packaging motif set forth as residues 17 to 26 of SEQ ID NO:21, and a C-terminal portion of HIV-1 p6 corresponding to amino acid residues 21 to 52 of SEQ ID NO:20 (designated SIV 17-23b; see US2013/0183334; Sunseri et al. (2011) J. Virol., 85:6263-6274). For example, in some embodiments, the hybrid or chimeric p6 domain comprises the sequence of amino acids set forth in SEQ ID NO:25.

3. Nucleic Acid Encoding a Heterologous Protein

In some embodiments, the viral vector contains a nucleic acid that encodes a heterologous recombinant protein. In some embodiments, the heterologous recombinant molecule is or includes a recombinant receptor, e.g. an antigen receptor, SB-transposons, e.g. for gene silencing, capsid-enclosed transposons, homologous double stranded nucleic acid, e.g. for genomic recombination or reporter genes (e.g. fluorescent proteins, such as GFP) or luciferase).

In some embodiments, the viral vector contains a nucleic acid that encodes a recombinant receptor and/or chimeric receptor, such as a heterologous receptor protein. The recombinant receptor, such as heterologous receptor, may include antigen receptors, such as functional non-TCR antigen receptors, including chimeric antigen receptors (CARs), and other antigen-binding receptors such as transgenic T cell receptors (TCRs). The receptors may also include other receptors, such as other chimeric receptors, such as receptors that bind to particular ligands and having transmembrane and/or intracellular signaling domains similar to those present in a CAR.

In any of such examples, the nucleic acid is inserted or located in a region of the viral vector, such as generally in a non-essential region of the viral genome. In some embodiments, the nucleic acid is inserted into the viral genome in the place of certain viral sequences to produce a virus that is replication defective.

In some embodiments, the encoded recombinant antigen receptor, e.g., CAR, is one that is capable of specifically binding to one or more ligand on a cell or disease to be targeted, such as a cancer, infectious disease, inflammatory or autoimmune disease, or other disease or condition, including those described herein for targeting with the provided methods and compositions.

In certain embodiments, an exemplary antigen is or includes otv36 integrin (avb6 integrin), B cell maturation antigen (BCMA), B7-H3, B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Ra), IL-13 receptor alpha 2 (IL-13Ra2), kinase insert domain receptor (kdr), kappa light chain, L cell adhesion molecule (L-CAM), CE7 epitope of L-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the exemplary antigens are orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, OEPHa2, ErbB2, 3, or 4, FBP, fetal acetylcholine receptor, GD2, GD3, HMW-MAA, IL-22R-alpha, IL-13R-alpha2, kdr, kappa light chain, Lewis Y, L-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by and/or characteristic of or specific for HIV, HCV, HBV, HPV, and/or other pathogens and/or oncogenic versions thereof.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

Antigen receptors, including CARs and recombinant TCRs, and production and introduction thereof, in some embodiments include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75.

a. Chimeric Antigen Receptors

In some embodiments, the nucleic acid contained in a genome of the viral vector encodes a chimeric antigen receptor (CAR). The CAR is generally a genetically engineered receptor with an extracellular ligand binding domain, such as an extracellular portion containing an antibody or fragment thereof, linked to one or more intracellular signaling components. In some embodiments, the chimeric antigen receptor includes a transmembrane domain and/or intracellular domain linking the extracellular domain and the intracellular signaling domain. Such molecules typically mimic or approximate a signal through a natural antigen receptor and/or signal through such a receptor in combination with a costimulatory receptor.

In some embodiments, CARs are constructed with a specificity for a particular marker, such as a marker expressed in a particular cell type to be targeted by adoptive therapy, e.g., a cancer marker and/or any of the antigens described. Thus, the CAR typically includes one or more antigen-binding fragment, domain, or portion of an antibody, or one or more antibody variable domains, and/or antibody molecules. In some embodiments, the CAR includes an antigen-binding portion or portions of an antibody molecule, such as a variable heavy chain (VH) or antigen-binding portion thereof, or a single-chain antibody fragment (scFv) derived from the variable heavy (VH) and variable light (VL) chains of a monoclonal antibody (mAb).

In some embodiments, engineered cells, such as T cells, are provided that express a CAR with specificity for a particular antigen (or marker or ligand), such as an antigen expressed on the surface of a particular cell type. In some embodiments, the antigen is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In particular embodiments, the recombinant receptor, such as chimeric receptor, contains an intracellular signaling region, which includes a cytoplasmic signaling domain or region (also interchangeably called an intracellular signaling domain or region), such as a cytoplasmic (intracellular) region capable of inducing a primary activation signal in a T cell, for example, a cytoplasmic signaling domain or region of a T cell receptor (TCR) component (e.g. a cytoplasmic signaling domain or region of a zeta chain of a CD3-zeta (CD3) chain or a functional variant or signaling portion thereof) and/or that comprises an immunoreceptor tyrosine-based activation motif (ITAM).

In some embodiments, the chimeric receptor further contains an extracellular ligand-binding domain that specifically binds to a ligand (e.g. antigen) antigen. In some embodiments, the chimeric receptor is a CAR that contains an extracellular antigen-recognition domain that specifically binds to an antigen. In some embodiments, the ligand, such as an antigen, is a protein expressed on the surface of cells. In some embodiments, the CAR is a TCR-like CAR and the antigen is a processed peptide antigen, such as a peptide antigen of an intracellular protein, which, like a TCR, is recognized on the cell surface in the context of a major histocompatibility complex (MHC) molecule.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the antibody or antigen-binding portion thereof is expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR. In some embodiments, the extracellular antigen binding domain specific for an MHC-peptide complex of a TCR-like CAR is linked to one or more intracellular signaling components, in some aspects via linkers and/or transmembrane domain(s). In some embodiments, such molecules can typically mimic or approximate a signal through a natural antigen receptor, such as a TCR, and, optionally, a signal through such a receptor in combination with a costimulatory receptor.

In some embodiments, the recombinant receptor, such as a chimeric receptor (e.g. CAR), includes a ligand-binding domain that binds, such as specifically binds, to an antigen (or a ligand). Among the antigens targeted by the chimeric receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas.

In some embodiments, the antigen (or a ligand) is a polypeptide. In some embodiments, it is a carbohydrate or other molecule. In some embodiments, the antigen (or a ligand) is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, the CAR contains an antibody or an antigen-binding fragment (e.g. scFv) that specifically recognizes an antigen, such as an intact antigen, expressed on the surface of a cell.

In some embodiments, the antigen (or a ligand) is a tumor antigen or cancer marker. In some embodiments, the antigen (or a ligand) the antigen is or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen _{1B} (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A1), Human leukocyte antigen A2 (HLA-A2), IL-22 receptor alpha(IL-22Ra), IL-13 receptor alpha 2 (IL-13Ra2), kinase insert domain receptor (kdr), kappa light chain, L cell adhesion molecule (L-CAM), CE7 epitope of L-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens. In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

Reference to “Major histocompatibility complex” (MHC) refers to a protein, generally a glycoprotein, that contains a polymorphic peptide binding site or binding groove that can, in some cases, complex with peptide antigens of polypeptides, including peptide antigens processed by the cell machinery. In some cases, MHC molecules can be displayed or expressed on the cell surface, including as a complex with peptide, i.e. MHC-peptide complex, for presentation of an antigen in a conformation recognizable by an antigen receptor on T cells, such as a TCRs or TCR-like antibody. Generally, MHC class I molecules are heterodimers having a membrane spanning a chain, in some cases with three a domains, and a non-covalently associated β2 microglobulin. Generally, MHC class II molecules are composed of two transmembrane glycoproteins, α and β, both of which typically span the membrane. An MHC molecule can include an effective portion of an MHC that contains an antigen binding site or sites for binding a peptide and the sequences necessary for recognition by the appropriate antigen receptor. In some embodiments, MHC class I molecules deliver peptides originating in the cytosol to the cell surface, where a MHC-peptide complex is recognized by T cells, such as generally CD8⁺ T cells, but in some cases CD4+ T cells. In some embodiments, MHC class II molecules deliver peptides originating in the vesicular system to the cell surface, where they are typically recognized by CD4⁺ T cells. Generally, MHC molecules are encoded by a group of linked loci, which are collectively termed H-2 in the mouse and human leukocyte antigen (HLA) in humans. Hence, typically human MHC can also be referred to as human leukocyte antigen (HLA).

The term “MHC-peptide complex” or “peptide-MHC complex” or variations thereof, refers to a complex or association of a peptide antigen and an MHC molecule, such as, generally, by non-covalent interactions of the peptide in the binding groove or cleft of the MHC molecule. In some embodiments, the MHC-peptide complex is present or displayed on the surface of cells. In some embodiments, the MHC-peptide complex can be specifically recognized by an antigen receptor, such as a TCR, TCR-like CAR or antigen-binding portions thereof.

In some embodiments, a peptide, such as a peptide antigen or epitope, of a polypeptide can associate with an MHC molecule, such as for recognition by an antigen receptor. Generally, the peptide is derived from or based on a fragment of a longer biological molecule, such as a polypeptide or protein. In some embodiments, the peptide typically is about 8 to about 24 amino acids in length. In some embodiments, a peptide has a length of from or from about 9 to 22 amino acids for recognition in the MHC Class II complex. In some embodiments, a peptide has a length of from or from about 8 to 13 amino acids for recognition in the MHC Class I complex. In some embodiments, upon recognition of the peptide in the context of an MHC molecule, such as MHC-peptide complex, the antigen receptor, such as TCR or TCR-like CAR, produces or triggers an activation signal to the T cell that induces a T cell response, such as T cell proliferation, cytokine production, a cytotoxic T cell response or other response.

In some embodiments, a TCR-like antibody or antigen-binding portion, are known or can be produced by known methods (see e.g. US Published Application Nos. US 2002/0150914; US 2003/0223994; US 2004/0191260; US 2006/0034850; US 2007/00992530; US20090226474; US20090304679; and International PCT Publication No. WO 03/068201).

In some embodiments, an antibody or antigen-binding portion thereof that specifically binds to a MHC-peptide complex, can be produced by immunizing a host with an effective amount of an immunogen containing a specific MHC-peptide complex. In some cases, the peptide of the MHC-peptide complex is an epitope of antigen capable of binding to the MHC, such as a tumor antigen, for example a universal tumor antigen, myeloma antigen or other antigen as described below. In some embodiments, an effective amount of the immunogen is then administered to a host for eliciting an immune response, wherein the immunogen retains a three-dimensional form thereof for a period of time sufficient to elicit an immune response against the three-dimensional presentation of the peptide in the binding groove of the MHC molecule. Serum collected from the host is then assayed to determine if desired antibodies that recognize a three-dimensional presentation of the peptide in the binding groove of the MHC molecule is being produced. In some embodiments, the produced antibodies can be assessed to confirm that the antibody can differentiate the MHC-peptide complex from the MHC molecule alone, the peptide of interest alone, and a complex of MHC and irrelevant peptide. The desired antibodies can then be isolated.

In some embodiments, an antibody or antigen-binding portion thereof that specifically binds to an MHC-peptide complex can be produced by employing antibody library display methods, such as phage antibody libraries. In some embodiments, phage display libraries of mutant Fab, scFv or other antibody forms can be generated, for example, in which members of the library are mutated at one or more residues of a CDR or CDRs. See e.g. US published application No. US20020150914, US2014/0294841; and Cohen C J. et al. (2003) J Mol. Recogn. 16:324-332.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

In some embodiments, the antigen-binding proteins, antibodies and antigen binding fragments thereof specifically recognize an antigen of a full-length antibody. In some embodiments, the heavy and light chains of an antibody can be full-length or can be an antigen-binding portion (a Fab, F(ab′)2, Fv or a single chain Fv fragment (scFv)). In other embodiments, the antibody heavy chain constant region is chosen from, e.g., IgG1, IgG2, IgG3, IgG4, IgM, IgA1, IgA2, IgD, and IgE, particularly chosen from, e.g., IgG, IgG2, IgG3, and IgG4, more particularly, IgG1 (e.g., human IgG). In another embodiment, the antibody light chain constant region is chosen from, e.g., kappa or lambda, particularly kappa.

Among the provided antibodies are antibody fragments. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (V_{H}) regions, single-chain antibody molecules such as scFvs and single-domain V_{H} single antibodies; and multispecific antibodies formed from antibody fragments. In particular embodiments, the antibodies are single-chain antibody fragments comprising a variable heavy chain region and/or a variable light chain region, such as scFvs.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Single-domain antibodies are antibody fragments comprising all or a portion of the heavy chain variable domain or all or a portion of the light chain variable domain of an antibody. In certain embodiments, a single-domain antibody is a human single-domain antibody. In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some embodiments, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody, refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Thus, in some embodiments, the chimeric antigen receptor, including TCR-like CARs, includes an extracellular portion containing an antibody or antibody fragment. In some embodiments, the antibody or fragment includes an scFv. In some aspects, the chimeric antigen receptor includes an extracellular portion containing the antibody or fragment and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM).

In some embodiments, the extracellular portion of the CAR, such as an antibody portion thereof, further includes a spacer, such as a spacer region between the antigen-recognition component, e.g. scFv, and a transmembrane domain. The spacer may be or include at least a portion of an immunoglobulin constant region or variant or modified version thereof, such as a hinge region, e.g., an IgG4 hinge region, and/or a CH1/CL and/or Fc region. In some embodiments, the recombinant receptor further comprises a spacer and/or a hinge region. In some embodiments, the constant region or portion is of a human IgG, such as IgG4 or IgG 1. In some aspects, the portion of the constant region serves as a spacer region between the antigen-recognition component, e.g., scFv, and transmembrane domain. In some embodiments, the spacer has the sequence set forth in SEQ ID NO: 27, and is encoded by the sequence set forth in SEQ ID NO: 28. In some embodiments, the spacer has the sequence set forth in SEQ ID NO: 29. In some embodiments, the spacer has the sequence set forth in SEQ ID NO: 30.

In some embodiments, the constant region or portion is of IgD. In some embodiments, the spacer has the sequence set forth in SEQ ID NO: 31. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 27, 29, 30 and 31.

In some embodiments, the spacer may be or include at least a portion of an immunoglobulin constant region or variant or modified version thereof, such as a hinge region, e.g., an IgG4 hinge region, and/or a C_{H}1/C_{L} and/or Fc region. In some embodiments, the recombinant receptor further comprises a spacer and/or a hinge region. In some embodiments, the constant region or portion is of a human IgG, such as IgG4 or IgG. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014/031687. In some embodiments, the spacer has the sequence set forth in SEQ ID NO: 27, and is encoded by the sequence set forth in SEQ ID NO: 28. In some embodiments, the spacer has the sequence set forth in SEQ ID NO: 29. In some embodiments, the spacer has the sequence set forth in SEQ ID NO: 30.

In some embodiments, the constant region or portion is of IgD. In some embodiments, the spacer has the sequence set forth in SEQ ID NO: 31. In some embodiments, the spacer has a sequence of amino acids that exhibits at least or at least about 85%, 86%, 87%, 88%, 89%, 90%, 91%, 92%, 93%, 94%, 95%, 96%, 97%, 98%, 99% or more sequence identity to any of SEQ ID NOS: 27, 29, 30 and 31.

The extracellular ligand binding, such as antigen recognition domain, generally is linked to one or more intracellular signaling components, such as signaling components that mimic activation through an antigen receptor complex, such as a TCR complex, in the case of a CAR, and/or signal via another cell surface receptor. In some embodiments, a transmembrane domain links the extracellular ligand binding and intracellular signaling domains. In some embodiments, the antigen binding component (e.g., antibody) is linked to one or more transmembrane and intracellular signaling regions. In some embodiments, the CAR includes a transmembrane domain fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 or CD154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The recombinant receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain and/or region or intracellular signaling domain and/or region of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling regions, e.g., comprising intracellular domain or domains, include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by at least two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR or CD3 zeta, FcR gamma, FcR beta, CD3 gamma, CD3 delta, CD3 epsilon, CD8, CD22, CD79a, CD79b, and CD66d. In certain embodiments, ITAM containing primary cytoplasmic signaling sequences include those derived from TCR or CD3 zeta, FcR gamma, or FcR beta. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, CD27, DAP10, and ICOS. In some aspects, the same CAR includes both the activating or signaling region and costimulatory components.

In some embodiments, the activating domain is included within one CAR, whereas the costimulatory component is provided by another CAR recognizing another antigen. In some embodiments, the CARs include activating or stimulatory CARs, and costimulatory CARs, both expressed on the same cell (see WO2014/055668). In some aspects, the CAR is the stimulatory or activating CAR; in other aspects, it is the costimulatory CAR. In some embodiments, the cells further include inhibitory CARs (iCARs, see Fedorov et al., Sci. Transl. Medicine, 5(215) (December, 2013), such as a CAR recognizing a different antigen, whereby an activating signal delivered through a CAR recognizing a first antigen is diminished or inhibited by binding of the inhibitory CAR to its ligand, e.g., to reduce off-target effects.

In certain embodiments, the intracellular signaling domain comprises a CD28 transmembrane and signaling domain linked to a CD3 intracellular domain. In some embodiments, the intracellular signaling domain comprises a chimeric CD28 and CD137 co-stimulatory domains, linked to a CD3 intracellular domain.

In some embodiments, the intracellular signaling domain of the CD8⁺ cytotoxic T cells is the same as the intracellular signaling domain of the CD4⁺ helper T cells. In some embodiments, the intracellular signaling domain of the CD8⁺ cytotoxic T cells is different than the intracellular signaling domain of the CD4⁺ helper T cells.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the recombinant receptor(s), e.g. CAR, encoded by nucleic acid(s) within the provided viral vectors further include one or more marker, e.g., for purposes of confirming transduction or engineering of the cell to express the receptor and/or selection and/or targeting of cells expressing molecule(s) encoded by the polynucleotide. In some aspects, such a marker may be encoded by a different nucleic acid or polynucleotide, which also may be introduced during the genetic engineering process, typically via the same method, e.g., transduction by any of the methods provided herein, e.g., via the same vector or type of vector.

In some aspects, the marker, e.g., transduction marker, is a protein and/or is a cell surface molecule. Exemplary markers are truncated variants of a naturally-occurring, e.g., endogenous markers, such as naturally-occurring cell surface molecules. In some aspects, the variants have reduced immunogenicity, reduced trafficking function, and/or reduced signaling function compared to the natural or endogenous cell surface molecule. In some embodiments, the marker is a truncated version of a cell surface receptor, such as truncated EGFR (tEGFR). In some aspects, the marker includes all or part (e.g., truncated form) of CD34, an NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A P2A, E2A and/or F2A. See, e.g., WO2014/031687.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular ligand-binding portion, such as an antigen-binding portion, such as an antibody or fragment thereof and in intracellular domain. In some embodiments, the antibody or fragment includes an scFv or a single-domain VH antibody and the intracellular domain contains an ITAM. In some aspects, the intracellular signaling domain includes a signaling domain of a zeta chain of a CD3-zeta (CD3L) chain. In some embodiments, the chimeric antigen receptor includes a transmembrane domain linking and/or disposed between the extracellular domain and the intracellular signaling region or domain.

In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1), or is a transmembrane domain that comprises the sequence of amino acids set forth in SEQ ID NO: 34 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 34; in some embodiments, the transmembrane-domain containing portion of the recombinant receptor comprises the sequence of amino acids set forth in SEQ ID NO: 35 or a sequence of amino acids having at least or at least about 85%, 86%, 87%, 88%, 89%, 90%, 91%, 92%, 93%, 94%, 95%, 96%, 97%, 98%, 99% or more sequence identity thereto.

In some embodiments, the chimeric antigen receptor contains an intracellular domain of a T cell costimulatory molecule. In some aspects, the T cell costimulatory molecule is CD28 or 4-1BB.

In some embodiments, the intracellular domain comprises an intracellular costimulatory signaling domain of human CD28 or functional variant or portion thereof, such as a 41 amino acid domain thereof and/or such a domain with an LL to GG substitution at positions 186-187 of a native CD28 protein. In some embodiments, the intracellular signaling region and/or domain can comprise the sequence of amino acids set forth in SEQ ID NO: 36 or 37 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 36 or 37. In some embodiments, the intracellular region and/or domain comprises an intracellular costimulatory signaling domain of 4-1BB or functional variant thereof, such as a 42-amino acid cytoplasmic domain of a human 4-1BB (Accession No. Q07011.1), or functional variant or portion thereof, such as the sequence of amino acids set forth in SEQ ID NO: 38 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 38.

In some embodiments, the intracellular signaling region and/or domain comprises a human CD3 chain, optionally a CD3 zeta stimulatory signaling domain or functional variant thereof, such as an 112 AA cytoplasmic domain of isoform 3 of human CD3t (Accession No.: P20963.2) or a CD3 zeta signaling domain as described in U.S. Pat. No. 7,446,190 or U.S. Pat. No. 8,911,993. In some embodiments, the intracellular signaling region comprises the sequence of amino acids set forth in SEQ ID NO: 39, 40, or 41 or a sequence of amino acids that exhibits at least or at least about 85%, 86%, 87%, 88%, 89%, 90%, 91%, 92%, 93%, 94%, 95%, 96%, 97%, 98%, 99% or more sequence identity to SEQ ID NO: 39, 40, or 41.

In some aspects, the spacer contains only a hinge region of an IgG, such as only a hinge of IgG4 or IgG1 such as the hinge only spacer set forth in SEQ ID NO: 27. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to C_{H2} and C_{H}3 domains, such as set forth in SEQ ID NO: 29. In some embodiments, the spacer is an Ig hinge, e.g., an IgG4 hinge, linked to a C_{H}3 domain only, such as set forth in SEQ ID NO: 30. In some embodiments, the spacer is or comprises a glycine-serine rich sequence or other flexible linker such as known flexible linkers.

For example, in some embodiments, the CAR includes: an extracellular ligand-binding portion, such as an antigen-binding portion, such as an antibody or fragment thereof, including sdAbs and scFvs, that specifically binds an antigen, e.g. an antigen described herein; a spacer such as any of the Ig-hinge containing spacers; a transmembrane domain that is a portion of CD28 or a variant thereof; an intracellular signaling domain containing a signaling portion of CD28 or functional variant thereof; and a signaling portion of CD3 zeta signaling domain or functional variant thereof. In some embodiments, the CAR includes: an extracellular ligand-binding portion, such as an antigen-binding portion, such as an antibody or fragment thereof, including sdAbs and scFvs, that specifically binds an antigen, e.g. an antigen described herein; a spacer such as any of the Ig-hinge containing spacers; a transmembrane domain that is a portion of CD28 or a variant thereof; an intracellular signaling domain containing a signaling portion of 4-1BB or functional variant thereof; and a signaling portion of CD3 zeta signaling domain or functional variant thereof. In some embodiments, such CAR constructs further includes a T2A ribosomal skip element and/or a tEGFR sequence, e.g., downstream of the CAR.

b. T Cell Receptors (TCRs)

In some embodiments, the recombinant molecule(s) encoded by the nucleic acid(s) is or include a recombinant T cell receptor (TCR). In some embodiments, the recombinant TCR is specific for an antigen, generally an antigen present on a target cell, such as a tumor-specific antigen, an antigen expressed on a particular cell type associated with an autoimmune or inflammatory disease, or an antigen derived from a viral pathogen or a bacterial pathogen. In some embodiments, engineered cells, such as T cells, are provided that express a TCR or antigen-binding portion thereof that recognizes an peptide epitope or T cell epitope of a target polypeptide, such as an antigen of a tumor, viral or autoimmune protein.

In some embodiments, a “T cell receptor” or “TCR” is a molecule that contains a variable α and β chains (also known as TCRα and TCRβ, respectively) or a variable γ and δ chains (also known as TCRα and TCRβ, respectively), or antigen-binding portions thereof, and which is capable of specifically binding to a peptide bound to an MHC molecule. In some embodiments, the TCR is in the αβ form. Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules.

Unless otherwise stated, the term “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions involved in recognition of the peptide, MHC and/or MHC-peptide complex.

In some embodiments, the variable domains of the TCR contain hypervariable loops, or complementarity determining regions (CDRs), which generally are the primary contributors to antigen recognition and binding capabilities and specificity. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCR molecules as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR3 is the main CDR responsible for antigen binding or specificity, or is the most important among the three CDRs on a given TCR variable region for antigen recognition, and/or for interaction with the processed peptide portion of the peptide-MHC complex. In some contexts, the CDR1 of the alpha chain can interact with the N-terminal part of certain antigenic peptides. In some contexts, CDR1 of the beta chain can interact with the C-terminal part of the peptide. In some contexts, CDR2 contributes most strongly to or is the primary CDR responsible for the interaction with or recognition of the MHC portion of the MHC-peptide complex. In some embodiments, the variable region of the P-chain can contain a further hypervariable region (CDR4 or HVR4), which generally is involved in superantigen binding and not antigen recognition (Kotb (1995) Clinical Microbiology Reviews, 8:411-426).

In some embodiments, a TCR also can contain a constant domain, a transmembrane domain and/or a short cytoplasmic tail (see, e.g., Janeway et al., Immunobiology: The Immune System in Health and Disease, 3rd Ed., Current Biology Publications, p. 4:33, 1997). In some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction.

In some embodiments, a TCR chain contains one or more constant domain. For example, the extracellular portion of a given TCR chain (e.g., α-chain or β-chain) can contain two immunoglobulin-like domains, such as a variable domain (e.g., Vα or Vβ; typically amino acids 1 to 116 based on Kabat numbering Kabat et al., “Sequences of Proteins of Immunological Interest, US Dept. Health and Human Services, Public Health Service National Institutes of Health, 1991, 5th ed.) and a constant domain (e.g., α-chain constant domain or Cα, typically positions 117 to 259 of the chain based on Kabat numbering or β chain constant domain or C_{β}, typically positions 117 to 295 of the chain based on Kabat) adjacent to the cell membrane. For example, in some cases, the extracellular portion of the TCR formed by the two chains contains two membrane-proximal constant domains, and two membrane-distal variable domains, which variable domains each contain CDRs. The constant domain of the TCR may contain short connecting sequences in which a cysteine residue forms a disulfide bond, thereby linking the two chains of the TCR. In some embodiments, a TCR may have an additional cysteine residue in each of the α and β chains, such that the TCR contains two disulfide bonds in the constant domains.

In some embodiments, the TCR chains contain a transmembrane domain. In some embodiments, the transmembrane domain is positively charged. In some cases, the TCR chain contains a cytoplasmic tail. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM that are involved in the signaling capacity of the TCR complex.

In some embodiments, the TCR may be a heterodimer of two chains α and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds.

In some embodiments, the TCR can be generated from a known TCR sequence(s), such as sequences of Vα,β chains, for which a substantially full-length coding sequence is readily available. Methods for obtaining full-length TCR sequences, including V chain sequences, from cell sources are well known. In some embodiments, nucleic acids encoding the TCR can be obtained from a variety of sources, such as by polymerase chain reaction (PCR) amplification of TCR-encoding nucleic acids within or isolated from a given cell or cells, or synthesis of publicly available TCR DNA sequences.

In some embodiments, the TCR is obtained from a biological source, such as from cells such as from a T cell (e.g. cytotoxic T cell), T-cell hybridomas or other publicly available source. In some embodiments, the T-cells can be obtained from in vivo isolated cells. In some embodiments, the TCR is a thymically selected TCR. In some embodiments, the TCR is a neoepitope-restricted TCR. In some embodiments, the T-cells can be a cultured T-cell hybridoma or clone. In some embodiments, the TCR or antigen-binding portion thereof or antigen-binding fragment thereof can be synthetically generated from knowledge of the sequence of the TCR.

In some embodiments, the TCR is generated from a TCR identified or selected from screening a library of candidate TCRs against a target polypeptide antigen, or target T cell epitope thereof. TCR libraries can be generated by amplification of the repertoire of Vα and Vβ from T cells isolated from a subject, including cells present in PBMCs, spleen or other lymphoid organ. In some cases, T cells can be amplified from tumor-infiltrating lymphocytes (TILs). In some embodiments, TCR libraries can be generated from CD4+ or CD8+ cells. In some embodiments, the TCRs can be amplified from a T cell source of a normal of healthy subject, i.e. normal TCR libraries. In some embodiments, the TCRs can be amplified from a T cell source of a diseased subject, i.e. diseased TCR libraries. In some embodiments, degenerate primers are used to amplify the gene repertoire of Vα and Vβ, such as by RT-PCR in samples, such as T cells, obtained from humans. In some embodiments, scTv libraries can be assembled from naïve Vα and Vβ libraries in which the amplified products are cloned or assembled to be separated by a linker. Depending on the source of the subject and cells, the libraries can be HLA allele-specific. Alternatively, in some embodiments, TCR libraries can be generated by mutagenesis or diversification of a parent or scaffold TCR molecule. In some aspects, the TCRs are subjected to directed evolution, such as by mutagenesis, e.g., of the α or β chain. In some aspects, particular residues within CDRs of the TCR are altered. In some embodiments, selected TCRs can be modified by affinity maturation. In some embodiments, antigen-specific T cells may be selected, such as by screening to assess CTL activity against the peptide. In some aspects, TCRs, e.g. present on the antigen-specific T cells, may be selected, such as by binding activity, e.g., particular affinity or avidity for the antigen.

In some embodiments, the genetically engineered antigen receptors include recombinant T cell receptors (TCRs) and/or TCRs cloned from naturally occurring T cells. In some embodiments, the TCR is one that has been cloned from naturally occurring T cells. In some embodiments, a high-affinity T cell clone for a target antigen (e.g., a cancer antigen) is identified and isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354. In some embodiments, the TCR or antigen-binding portion thereof is one that has been modified or engineered. In some embodiments, directed evolution methods are used to generate TCRs with altered properties, such as with higher affinity for a specific MHC-peptide complex. In some embodiments, directed evolution is achieved by display methods including, but not limited to, yeast display (Holler et al. (2003) Nat Immunol, 4, 55-62; Holler et al. (2000) Proc Natl Acad Sci USA, 97, 5387-92), phage display (Li et al. (2005) Nat Biotechnol, 23, 349-54), or T cell display (Chervin et al. (2008) J Immunol Methods, 339, 175-84). In some embodiments, display approaches involve engineering, or modifying, a known, parent or reference TCR. For example, in some cases, a wild-type TCR can be used as a template for producing mutagenized TCRs in which in one or more residues of the CDRs are mutated, and mutants with an desired altered property, such as higher affinity for a desired target antigen, are selected.

In some embodiments, peptides of a target polypeptide for use in producing or generating a TCR of interest are known or can be readily identified by a skilled artisan. In some embodiments, peptides suitable for use in generating TCRs or antigen-binding portions can be determined based on the presence of an HLA-restricted motif in a target polypeptide of interest, such as a target polypeptide described below. In some embodiments, peptides are identified using available computer prediction models. In some embodiments, for predicting MHC class I binding sites, such models include, but are not limited to, ProPred1 (Singh and Raghava (2001) Bioinformatics 17(12):1236-1237, and SYFPEITHI (see Schuler et al. (2007) Immunoinformatics Methods in Molecular Biology, 409(1): 75-93 2007). In some embodiments, the MHC-restricted epitope is HLA-A0201, which is expressed in approximately 39-46% of all Caucasians and therefore, represents a suitable choice of MHC antigen for use preparing a TCR or other MHC-peptide binding molecule.

HLA-A0201-binding motifs and the cleavage sites for proteasomes and immune-proteasomes using computer prediction models are known. For predicting MHC class I binding sites, such models include, but are not limited to, ProPred1 (described in more detail in Singh and Raghava, ProPred: prediction of HLA-DR binding sites. BIOINFORMATICS 17(12):1236-1237 2001), and SYFPEITHI (see Schuler et al. SYFPEITHI, Database for Searching and T-Cell Epitope Prediction. in Immunoinformatics Methods in Molecular Biology, vol 409(1): 75-93 2007).

In some embodiments, the TCR or antigen binding portion thereof may be a recombinantly produced natural protein or mutated form thereof in which one or more property, such as binding characteristic, has been altered. In some embodiments, a TCR may be derived from one of various animal species, such as human, mouse, rat, or other mammal. A TCR may be cell-bound or in soluble form. In some embodiments, for purposes of the provided methods, the TCR is in cell-bound form expressed on the surface of a cell.

In some embodiments, the TCR is a full-length TCR. In some embodiments, the TCR is an antigen-binding portion. In some embodiments, the TCR is a dimeric TCR (dTCR). In some embodiments, the TCR is a single-chain TCR (sc-TCR). In some embodiments, a dTCR or scTCR have the structures as described in WO 03/020763, WO 04/033685, WO2011/044186.

In some embodiments, the TCR contains a sequence corresponding to the transmembrane sequence. In some embodiments, the TCR does contain a sequence corresponding to cytoplasmic sequences. In some embodiments, the TCR is capable of forming a TCR complex with CD3. In some embodiments, any of the TCRs, including a dTCR or scTCR, can be linked to signaling domains that yield an active TCR on the surface of a T cell. In some embodiments, the TCR is expressed on the surface of cells.

In some embodiments a dTCR contains a first polypeptide wherein a sequence corresponding to a TCR α chain variable region sequence is fused to the N terminus of a sequence corresponding to a TCR α chain constant region extracellular sequence, and a second polypeptide wherein a sequence corresponding to a TCR β chain variable region sequence is fused to the N terminus a sequence corresponding to a TCR β chain constant region extracellular sequence, the first and second polypeptides being linked by a disulfide bond. In some embodiments, the bond can correspond to the native inter-chain disulfide bond present in native dimeric αβ TCRs. In some embodiments, the interchain disulfide bonds are not present in a native TCR. For example, in some embodiments, one or more cysteines can be incorporated into the constant region extracellular sequences of dTCR polypeptide pair. In some cases, both a native and a non-native disulfide bond may be desirable. In some embodiments, the TCR contains a transmembrane sequence to anchor to the membrane.

In some embodiments, a dTCR contains a TCR α chain containing a variable α domain, a constant α domain and a first dimerization motif attached to the C-terminus of the constant α domain, and a TCR β chain comprising a variable β domain, a constant β domain and a first dimerization motif attached to the C-terminus of the constant β domain, wherein the first and second dimerization motifs easily interact to form a covalent bond between an amino acid in the first dimerization motif and an amino acid in the second dimerization motif linking the TCR α chain and TCR β chain together.

In some embodiments, the TCR is a scTCR. Typically, a scTCR can be generated using methods known, See e.g., Soo Hoo, W. F. et al. PNAS (USA) 89, 4759 (1992); Wiilfing, C. and Plückthun, A., J. Mol. Biol. 242, 655 (1994); Kurucz, I. et al. PNAS (USA) 90 3830 (1993); International published PCT Nos. WO 96/13593, WO 96/18105, WO99/60120, WO99/18129, WO 03/020763, WO2011/044186; and Schlueter, C. J. et al. J. Mol. Biol. 256, 859 (1996). In some embodiments, a scTCR contains an introduced non-native disulfide interchain bond to facilitate the association of the TCR chains (see e.g. International published PCT No. WO 03/020763). In some embodiments, a scTCR is a non-disulfide linked truncated TCR in which heterologous leucine zippers fused to the C-termini thereof facilitate chain association (see e.g. International published PCT No. WO99/60120). In some embodiments, a scTCR contain a TCRα variable domain covalently linked to a TCRβ variable domain via a peptide linker (see e.g., International published PCT No. WO99/18129).

In some embodiments, a scTCR contains a first segment constituted by an amino acid sequence corresponding to a TCR α chain variable region, a second segment constituted by an amino acid sequence corresponding to a TCR β chain variable region sequence fused to the N terminus of an amino acid sequence corresponding to a TCR β chain constant domain extracellular sequence, and a linker sequence linking the C terminus of the first segment to the N terminus of the second segment.

In some embodiments, a scTCR contains a first segment constituted by an a chain variable region sequence fused to the N terminus of an a chain extracellular constant domain sequence, and a second segment constituted by a P chain variable region sequence fused to the N terminus of a sequence β chain extracellular constant and transmembrane sequence, and, optionally, a linker sequence linking the C terminus of the first segment to the N terminus of the second segment.

In some embodiments, a scTCR contains a first segment constituted by a TCR β chain variable region sequence fused to the N terminus of a P chain extracellular constant domain sequence, and a second segment constituted by an a chain variable region sequence fused to the N terminus of a sequence a chain extracellular constant and transmembrane sequence, and, optionally, a linker sequence linking the C terminus of the first segment to the N terminus of the second segment.

In some embodiments, the linker of a scTCRs that links the first and second TCR segments can be any linker capable of forming a single polypeptide strand, while retaining TCR binding specificity. In some embodiments, the linker sequence may, for example, have the formula —P-AA-P— wherein P is proline and AA represents an amino acid sequence wherein the amino acids are glycine and serine. In some embodiments, the first and second segments are paired so that the variable region sequences thereof are orientated for such binding. Hence, in some cases, the linker has a sufficient length to span the distance between the C terminus of the first segment and the N terminus of the second segment, or vice versa, but is not too long to block or reduces bonding of the scTCR to the target ligand. In some embodiments, the linker can contain from or from about 10 to 45 amino acids, such as 10 to 30 amino acids or 26 to 41 amino acids residues, for example 29, 30, 31 or 32 amino acids. In some embodiments, the linker has the formula —PGGG-(SGGGG)₅-P— wherein P is proline, G is glycine and S is serine (SEQ ID NO: 48). In some embodiments, the linker has the sequence GSADDAKKDAAKKDGKS (SEQ ID NO: 49)

In some embodiments, the scTCR contains a covalent disulfide bond linking a residue of the immunoglobulin region of the constant domain of the α chain to a residue of the immunoglobulin region of the constant domain of the β chain. In some embodiments, the interchain disulfide bond in a native TCR is not present. For example, in some embodiments, one or more cysteines can be incorporated into the constant region extracellular sequences of the first and second segments of the scTCR polypeptide. In some cases, both a native and a non-native disulfide bond may be desirable.

In some embodiments of a dTCR or scTCR containing introduced interchain disulfide bonds, the native disulfide bonds are not present. In some embodiments, the one or more of the native cysteines forming a native interchain disulfide bonds are substituted to another residue, such as to a serine or alanine. In some embodiments, an introduced disulfide bond can be formed by mutating non-cysteine residues on the first and second segments to cysteine. Exemplary non-native disulfide bonds of a TCR are described in published International PCT No. WO2006/000830.

In some embodiments, the TCR or antigen-binding fragment thereof exhibits an affinity with an equilibrium binding constant for a target antigen of between or between about 10−5 and 10−12 M and all individual values and ranges therein. In some embodiments, the target antigen is an MHC-peptide complex or ligand.

In some embodiments, nucleic acid or nucleic acids encoding a TCR, such as α and β chains, can be amplified by PCR, cloning or other suitable means and cloned into a suitable expression vector or vectors. The expression vector can be any suitable recombinant expression vector, and can be used to transform or transfect any suitable host. Suitable vectors include those designed for propagation and expansion or for expression or both, such as plasmids and viruses.

In some embodiments, the vector can be a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some cases, bacteriophage vectors, such as XG10, XGT11, XZapII (Stratagene), XEMBL4, and XNM1149, also can be used. In some embodiments, plant expression vectors can be used and include pBIOl, pBI101.2, pBI101.3, pBI121 and pBIN19 (Clontech). In some embodiments, animal expression vectors include pEUK-C1, pMAM and pMAMneo (Clontech). In some embodiments, a viral vector is used, such as a retroviral vector.

In some embodiments, the recombinant expression vectors can be prepared using standard recombinant DNA techniques. In some embodiments, vectors can contain regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based. In some embodiments, the vector can contain a nonnative promoter operably linked to the nucleotide sequence encoding the TCR or antigen-binding portion (or other MHC-peptide binding molecule). In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other known promoters also are contemplated.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpression. In some embodiments, the nucleic acid encoding a TCR further includes a marker to confirm transduction or engineering of the cell to express the receptor. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683.

In some embodiments, to generate a vector encoding a TCR, the α and β chains are PCR amplified from total cDNA isolated from a T cell clone expressing the TCR of interest and cloned into an expression vector. In some embodiments, the α and β chains are cloned into the same vector. In some embodiments, the α and β chains are cloned into different vectors. In some embodiments, the generated α and β chains are incorporated into a retroviral, e.g. lentiviral, vector.

c. Chimeric Auto-Antibody Receptor (CAAR)

In some embodiments, the recombinant receptor is a chimeric autoantibody receptor (CAAR). In some embodiments, the CAAR is specific for an autoantibody. In some embodiments, a cell expressing the CAAR, such as a T cell engineered to express a CAAR, can be used to specifically bind to and kill autoantibody-expressing cells, but not normal antibody expressing cells. In some embodiments, CAAR-expressing cells can be used to treat an autoimmune disease associated with expression of self-antigens, such as autoimmune diseases. In some embodiments, CAAR-expressing cells can target B cells that ultimately produce the autoantibodies and display the autoantibodies on their cell surfaces, mark these B cells as disease-specific targets for therapeutic intervention. In some embodiments, CAAR-expressing cells can be used to efficiently targeting and killing the pathogenic B cells in autoimmune diseases by targeting the disease-causing B cells using an antigen-specific chimeric autoantibody receptor. In some embodiments, the recombinant receptor is a CAAR, such as any described in U.S. Patent Application Pub. No. US 2017/0051035.

In some embodiments, the CAAR comprises an autoantibody binding domain, a transmembrane domain, and an intracellular signaling region. In some embodiments, the intracellular signaling region comprises an intracellular signaling domain. In some embodiments, the intracellular signaling domain is or comprises a primary signaling domain, a signaling domain that is capable of inducing a primary activation signal in a T cell, a signaling domain of a T cell receptor (TCR) component, and/or a signaling domain comprising an immunoreceptor tyrosine-based activation motif (ITAM). In some embodiments, the intracellular signaling region comprises a secondary or costimulatory signaling region (secondary intracellular signaling regions).

In some embodiments, the autoantibody binding domain comprises an autoantigen or a fragment thereof. The choice of autoantigen can depend upon the type of autoantibody being targeted. For example, the autoantigen may be chosen because it recognizes an autoantibody on a target cell, such as a B cell, associated with a particular disease state, e.g. an autoimmune disease, such as an autoantibody-mediated autoimmune disease. In some embodiments, the autoimmune disease includes pemphigus vulgaris (PV). Exemplary autoantigens include desmoglein 1 (Dsg1) and Dsg3.

d. Multi-Targeting

c. In some embodiments, the cells and methods include multi-targeting strategies, such as expression of two or more genetically engineered receptors on the cell, each recognizing the same of a different antigen and typically each including a different intracellular signaling component. Such multi-targeting strategies are described, for example, in International Patent Application Publication No: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating or stimulating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating or stimulating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-κB and/or AP-1, and/or induction of gene expression of factors such as cytokines, proliferation, and/or survival.

In some embodiments, the first and/or second receptor includes intracellular signaling domains of costimulatory receptors such as CD28, CD137 (4-1BB), OX40, and/or ICOS. In some embodiments, the first and second receptor include an intracellular signaling domain of a costimulatory receptor that are different. In one embodiment, the first receptor contains a CD28 costimulatory signaling region and the second receptor contain a 4-1BB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating or stimulating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

e. Other Regulatory Elements

In some embodiments of the methods and compositions provided herein, the nucleic acid sequence contained in the viral vector genome encoding an recombinant receptor, such as an antigen receptor, for example a CAR, is operably linked in a functional relationship with other genetic elements, for example transcription regulatory sequences including promoters or enhancers, to regulate expression of the sequence of interest in a particular manner. In certain instances, such transcriptional regulatory sequences are those that are temporally and/or spatially regulated with respect to activity. Expression control elements that can be used for regulating the expression of the components are known and include, but are not limited to, inducible promoters, constitutive promoters, secretion signals, enhancers and other regulatory elements. In some embodiments, the nucleic acid sequence contained in the viral vector genome contain multiple expression control elements that control different encoded components, e.g., different receptor components and/or signaling components, such that the expression, function and/or activity of the recombinant receptor and/or the engineered cell, e.g. cell expressing the engineered receptor, can be regulated, e.g., are inducible, repressible, regulatable and/or user controlled. In some embodiments, one or more vectors can contain one or more nucleic acid sequences that contain one or more expression control elements and/or one or more encoded components, such that the nucleic acid sequences together can regulate the expression, activity and/or function of the encoded components, e.g., recombinant receptor, or the engineered cell.

In some embodiments, the nucleic acid sequence encoding a recombinant receptor, such as an antigen receptor, for example a CAR, is operably linked with internal promoter/enhancer regulatory sequences. The promoters employed may be constitutive, tissue-specific, inducible, and/or useful under the appropriate conditions to direct high level expression of the introduced DNA segment. The promoter may be heterologous or endogenous. In some embodiments, a promoter and/or enhancer is produced synthetically. In some embodiments, a promoter and/or enhancer is produced using recombinant cloning and/or nucleic acid amplification technology.

In some cases, the nucleic acid sequence encoding the recombinant receptor contains a signal sequence that encodes a signal peptide. In some aspects, the signal sequence may encode a signal peptide derived from a native polypeptide. In other aspects, the signal sequence may encode a heterologous or non-native signal peptide, such as the exemplary signal peptide of the GMCSFR alpha chain set forth in SEQ ID NO: 51 and encoded by the nucleotide sequence set forth in SEQ ID NO: 50. In some cases, the nucleic acid sequence encoding the recombinant receptor, e.g., chimeric antigen receptor (CAR) contains a signal sequence that encodes a signal peptide. Non-limiting exemplary examples of signal peptides include, for example, the GMCSFR alpha chain signal peptide set forth in SEQ ID NO: 51 and encoded by the nucleotide sequence set forth in SEQ ID NO: 50, or the CD8 alpha signal peptide set forth in SEQ ID NO:52.

In some embodiments, the polynucleotide encoding the recombinant receptor contains at least one promoter that is operatively linked to control expression of the recombinant receptor. In some examples, the polynucleotide contains two, three, or more promoters operatively linked to control expression of the recombinant receptor.

In certain cases where nucleic acid molecules encode two or more different polypeptide chains, e.g., a recombinant receptor and a marker, each of the polypeptide chains can be encoded by a separate nucleic acid molecule. For example, two separate nucleic acids are provided, and each can be individually transferred or introduced into the cell for expression in the cell. In some embodiments, the nucleic acid encoding the recombinant receptor and the nucleic acid encoding the marker are operably linked to the same promoter and are optionally separated by an internal ribosome entry site (IRES), or a nucleic acid encoding a self-cleaving peptide or a peptide that causes ribosome skipping, which optionally is a T2A, a P2A, a E2A or a F2A. In some embodiments, the nucleic acids encoding the marker and the nucleic acid encoding the recombinant receptor are operably linked to two different promoters. In some embodiments, the nucleic acid encoding the marker and the nucleic acid encoding the recombinant receptor are present or inserted at different locations within the genome of the cell. In some embodiments, the polynucleotide encoding the recombinant receptor is introduced into a composition containing cultured cells, such as by retroviral transduction, transfection, or transformation.

In some embodiments, such as those where the polynucleotide contains a first and second nucleic acid sequence, the coding sequences encoding each of the different polypeptide chains can be operatively linked to a promoter, which can be the same or different. In some embodiments, the nucleic acid molecule can contain a promoter that drives the expression of two or more different polypeptide chains. In some embodiments, such nucleic acid molecules can be multicistronic (bicistronic or tricistronic, see e.g., U.S. Pat. No. 6,060,273). In some embodiments, transcription units can be engineered as a bicistronic unit containing an IRES (internal ribosome entry site), which allows coexpression of gene products ((e.g. encoding the marker and encoding the recombinant receptor) by a message from a single promoter. Alternatively, in some cases, a single promoter may direct expression of an RNA that contains, in a single open reading frame (ORF), two or three genes (e.g. encoding the marker and encoding the recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as a T2A, can cause the ribosome to skip (ribosome skipping) synthesis of a peptide bond at the C-terminus of a 2A element, leading to separation between the end of the 2A sequence and the next peptide downstream (see, for example, de Felipe, Genetic Vaccines and Ther. 2:13 (2004) and de Felipe et al. Traffic 5:616-626 (2004)). Various 2A elements are known. Examples of 2A sequences that can be used in the methods and system disclosed herein, without limitation, 2A sequences from the foot-and-mouth disease virus (F2A, e.g., SEQ ID NO: 47), equine rhinitis A virus (E2A, e.g., SEQ ID NO: 46), Thosea asigna virus (T2A, e.g., SEQ ID NO: 32 or 43), and porcine teschovirus-1 (P2A, e.g., SEQ ID NO: 44 or 45) as described in U.S. Patent Publication No. 20070116690.

Any of the recombinant receptors described herein can be encoded by polynucleotides containing one or more nucleic acid sequences encoding recombinant receptors, in any combinations or arrangements. For example, one, two, three or more polynucleotides can encode one, two, three or more different polypeptides, e.g., recombinant receptors. In some embodiments, one vector or construct contains a nucleic acid sequence encoding marker, and a separate vector or construct contains a nucleic acid sequence encoding a recombinant receptor, e.g., CAR. In some embodiments, the nucleic acid encoding the marker and the nucleic acid encoding the recombinant receptor are operably linked to two different promoters. In some embodiments, the nucleic acid encoding the recombinant receptor is present downstream of the nucleic acid encoding the marker.

In some embodiments, the vector backbone contains a nucleic acid sequence encoding one or more marker(s). In some embodiments, the one or more marker(s) is a transduction marker, surrogate marker and/or a selection marker.

In some embodiments, the marker is a transduction marker or a surrogate marker. A transduction marker or a surrogate marker can be used to detect cells that have been introduced with the polynucleotide, e.g., a polynucleotide encoding a recombinant receptor. In some embodiments, the transduction marker can indicate or confirm modification of a cell. In some embodiments, the surrogate marker is a protein that is made to be co-expressed on the cell surface with the recombinant receptor, e.g. CAR. In particular embodiments, such a surrogate marker is a surface protein that has been modified to have little or no activity. In certain embodiments, the surrogate marker is encoded on the same polynucleotide that encodes the recombinant receptor. In some embodiments, the nucleic acid sequence encoding the recombinant receptor is operably linked to a nucleic acid sequence encoding a marker, optionally separated by an internal ribosome entry site (IRES), or a nucleic acid encoding a self-cleaving peptide or a peptide that causes ribosome skipping, such as a 2A sequence, such as a T2A, a P2A, a E2A or a F2A. Extrinsic marker genes may in some cases be utilized in connection with engineered cell to permit detection or selection of cells and, in some cases, also to promote cell suicide.

Exemplary surrogate markers can include truncated cell surface polypeptides, such as a truncated human epidermal growth factor receptor 2 (tHER2), a truncated epidermal growth factor receptor (EGFRt, exemplary EGFRt sequence set forth in SEQ ID NO: 33 or 42) or a prostate-specific membrane antigen (PSMA) or modified form thereof. EGFRt may contain an epitope recognized by the antibody cetuximab (Erbitux®) or other therapeutic anti-EGFR antibody or binding molecule, which can be used to identify or select cells that have been engineered with the EGFRt construct and a recombinant receptor, such as a chimeric antigen receptor (CAR), and/or to eliminate or separate cells expressing the receptor. See U.S. Pat. No. 8,802,374 and Liu et al., Nature Biotech. 2016 April; 34(4): 430-434). In some aspects, the marker, e.g. surrogate marker, includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in PCT Pub. No. WO2014031687. For example, the marker can be a truncated EGFR (tEGFR) that is, optionally, linked to a linker sequence, such as a T2A cleavable linker sequence. An exemplary polypeptide for a truncated EGFR (e.g. tEGFR) comprises the sequence of amino acids set forth in SEQ ID NO: 33 or 42 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 33 or 42.

In some embodiments, the marker is or comprises a fluorescent protein, such as green fluorescent protein (GFP), enhanced green fluorescent protein (EGFP), such as super-fold GFP, red fluorescent protein (RFP), such as tdTomato, mCherry, mStrawberry, AsRed2, DsRed or DsRed2, cyan fluorescent protein (CFP), blue green fluorescent protein (BFP), enhanced blue fluorescent protein (EBFP), and yellow fluorescent protein (YFP), and variants thereof, including species variants, monomeric variants, and codon-optimized and/or enhanced variants of the fluorescent proteins. In some embodiments, the marker is or comprises an enzyme, such as a luciferase, the lacZ gene from E. coli, alkaline phosphatase, secreted embryonic alkaline phosphatase (SEAP), chloramphenicol acetyl transferase (CAT). Exemplary light-emitting reporter genes include luciferase (luc), P-galactosidase, chloramphenicol acetyltransferase (CAT), β-glucuronidase (GUS) or variants thereof.

In some embodiments, the marker is a selection marker. In some embodiments, the selection marker is or comprises a polypeptide that confers resistance to exogenous agents or drugs. In some embodiments, the selection marker is an antibiotic resistance gene. In some embodiments, the selection marker is an antibiotic resistance gene confers antibiotic resistance to a mammalian cell. In some embodiments, the selection marker is or comprises a Puromycin resistance gene, a Hygromycin resistance gene, a Blasticidin resistance gene, a Neomycin resistance gene, a Geneticin resistance gene or a Zeocin resistance gene or a modified form thereof.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments a promoter and/or enhancer may be one that is naturally associated with a nucleic acid sequence, as may be obtained by isolating the 5′ non-coding sequences located upstream of the coding segment and/or exon. Alternatively, in some embodiments the coding nucleic acid segment may be positioned under the control of a recombinant and/or heterologous promoter and/or enhancer, which is not normally associated with the coding nucleic acid sequence in the natural setting. For example, exemplary promoters used in recombinant DNA construction include, but are not limited to, the β-lactamase (penicillinase), lactose, tryptophan (trp), RNA polymerase (pol) III promoters including, the human and murine U6 pol III promoters as well as the human and murine H1 RNA pol III promoters; RNA polymerase (pol) II promoters; cytomegalovirus immediate early promoter (pCMV), elongation factor-1 alpha (EF-1 alpha), and the Rous Sarcoma virus long terminal repeat promoter (pRSV) promoter systems. In some embodiments, the promoter may be obtained, for example, from the genomes of viruses such as polyoma virus, fowlpox virus, adenovirus, bovine papilloma virus, avian sarcoma virus, cytomegalovirus, a retrovirus, hepatitis-B virus and Simian Virus 40 (SV40). The promoter may also be, for example, a heterologous mammalian promoter, e.g., the actin promoter or an immunoglobulin promoter, a heat-shock promoter, or the promoter normally associated with the native sequence, provided such promoters are compatible with the target cell. In one embodiment, the promoter is the naturally occurring viral promoter in a viral expression system.

In some embodiments, the promoter may be constitutively active. Non-limiting examples of constitutive promoters that may be used include the promoter for ubiquitin (U.S. Pat. No. 5,510,474; WO 98/32869), CMV (Thomsen et al., PNAS 81:659, 1984; U.S. Pat. No. 5,168,062), beta-actin (Gunning et al. 1989 Proc. Natl. Acad. Sci. USA 84:4831-4835) and pgk (see, for example, Adra et al. 1987 Gene 60:65-74; Singer-Sam et al. 1984 Gene 32:409-417; and Dobson et al. 1982 Nucleic Acids Res. 10:2635-2637).

In some embodiments, the promoter may be a tissue specific promoter and/or a target cell-specific promoter. In some embodiments, the promoters may be selected to allow for inducible expression of the sequence of interest. A number of systems for inducible expression are known, including the tetracycline responsive system, the lac operator-repressor system, as well as promoters responsive to a variety of environmental or physiological changes, including heat shock, metal ions, such as metallothionein promoter, interferons, hypoxia, steroids, such as progesterone or glucocorticoid receptor promoter, radiation, such as VEGF promoter. In some embodiments, the tetracycline-(tet)-regulatable system, which is based on the inhibitory action of the tet repression (tetr) of Escherichia coli on the tet operator sequence (TECO), can be modified for use in mammalian systems and used as a regulatable element for expression cassettes. These systems are well known. (See, Goshen and Badgered, Proc. Natl. Acad. Sci. USA 89: 5547-51 (1992), Shockett et al., Proc. Natl. Acad. Sci. USA 92:6522-26 (1996), Lindemann et al., Mol. Med. 3:466-76 (1997)).

A combination of promoters may also be used to obtain the desired expression of the gene of interest. The artisan of ordinary skill will be able to select a promoter based on the desired expression pattern of the gene in the organism or the target cell of interest.

In some embodiments, an enhancer may also be present in the viral construct to increase expression of the gene of interest. Enhancers are typically cis-acting nucleic acid elements, usually about 10 to 300 by in length, that act on a promoter to increase its transcription. Many enhancers in viral genomes, such as HIV or CMV are known. For example, the CMV enhancer (Boshart et al. Cell, 41:521, 1985) can be used. Other examples include, for example, the SV40 enhancer on the late side of the replication origin (bp 100-270), the cytomegalovirus early promoter enhancer, the polyoma enhancer on the late side of the replication origin, and adenovirus enhancers. In some cases, an enhancer is from a mammalian gene, such as an enhancer from a globin, elastase, albumin, alpha-fetoprotein or insulin). An enhancer can be used in combination with a heterologous promoter. The enhancer may be spliced into the vector at a position 5′ or 3′ to the polynucleotide sequence encoding the gene of interest, but is generally located at a site 5′ from the promoter. One of ordinary skill in the art will be able to select the appropriate enhancer based on the desired expression pattern.

The viral vector genome may also contain additional genetic elements. The types of elements that can be included in the constructs are not limited in any way and can be chosen by one with skill in the art.

For example, a signal that facilitates nuclear entry of the viral genome in the target cell may be included. An example of such a signal is the HIV-1 flap signal (in some cases referred to as the flap sequence). In addition, the vector genome may contain one or more genetic elements designed to enhance expression of the gene of interest. In some embodiments, the genome contains a post-transcriptional regulatory element (PRE) or modified form thereof that exhibits post-transcriptional activity. For example, in some embodiments, a woodchuck hepatitis virus posts-transcriptional responsive element (WPRE) may be placed into the construct (Zufferey et al. 1999. J. Virol. 74:3668-3681; Deglon et al. 2000. Hum. Gene Ther. 11:179-190). In some embodiments, the vector genome lacks a flap sequence and/or lacks a WPRE. In some embodiments, the vector genome contains a mutated or defective flap sequence and/or WPRE.

In some instances, more than one open reading frame encoding separate heterologous proteins can be included. For example, in some embodiments, if a reporter and/or detectable and/or selectable gene is included in the expression construct, an internal ribosomal entry site (IRES) sequence can be included. Typically, the additional genetic elements are operably linked with and controlled by an independent promoter/enhancer. The additional genetic element can be a reporter gene, a selectable marker or other desired gene.

In some embodiments, other various regulatory elements can include a transcription initiation region and/or a termination region. Expression vectors may also contain sequences for the termination of transcription and for stabilizing the mRNA. Such sequences are known and are often found naturally in the 5′ and, occasionally 3′, untranslated regions of eukaryotic or viral DNAs or cDNAs. Examples of transcription termination region include, but are not limited to, polyadenylation signal sequences. Examples of polyadenylation signal sequences include, but are not limited to, Bovine growth hormone (BGH) poly(A), SV40 late poly(A), rabbit beta-globin (RBG) poly(A), thymidine kinase (TK) poly(A) sequences, and any variants thereof.

In some embodiments, regulatory elements can include regulatory elements and/or systems that allow regulatable expression and/or activity of the recombinant receptor, e.g., CAR. In some embodiments, regulatable expression and/or activity is achieved by configuring the recombinant receptor to contain or be controlled by particular regulatory elements and/or systems. In some embodiments, one or more additional receptors can be used in an expression regulation systems. In some embodiments, expression regulation systems can include systems that require exposure to or binding of a specific ligand that can regulate the expression and/or activity of the recombinant receptor. In some embodiments, regulated expression of the recombinant receptor, e.g., CAR, is achieved a regulatable transcription factor release system, e.g., a modified Notch signaling system (see, e.g., Roybal et al., Cell (2016) 164:770-779; Morsut et al., Cell (2016) 164:780-791). In some embodiments, regulation of activity of the recombinant receptor is achieved by administration of an additional agent that can induce conformational changes and/or multimerization of polypeptides, e.g., the recombinant receptor. In some embodiments, the additional agent is a chemical inducer (see, e.g., U.S. Patent Publication No. 2016/0046700, Clackson et al. (1998) Proc Natl Acad Sci USA. 95(18):10437-42; Spencer et al. (1993) Science 262(5136):1019-24; Farrar et al. (1996) Nature 383 (6596):178-81; Miyamoto et al. (2012) Nature Chemical Biology 8(5): 465-70; Erhart et al. (2013) Chemistry and Biology 20(4): 549-57).

4. Preparation of Viral Vector Particles

The viral vector genome is typically constructed in a plasmid form that can be transfected into a packaging or producer cell line. Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g. vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g. antigen receptor, such as CAR, can be detected.

C. Incubation

In some embodiments, the provided methods involve methods of transducing cells by contacting, e.g. incubating, a cell composition comprising a plurality of cells (hereinafter also called an “input composition”) with a (1) a viral particle. In some embodiments, the input composition is or comprises primary cells obtained from a subject, such as cells enriched and/or selected from a subject.

In some embodiments, the input composition comprises primary cells obtained from a subject. In some aspects, the sample is a whole blood sample, a buffy coat sample, a peripheral blood mononuclear cells (PBMC) sample, an unfractionated T cell sample, a lymphocyte sample, a white blood cell sample, an apheresis product, or a leukapheresis product.

In some embodiments, prior to the selection and/or transduction of cells, the sample containing primary cells is contacted ex vivo with or contains serum or plasma at a concentration of at least or at least about 10% (v/v), at least or at least about 15% (v/v), at least or at least about 20% (v/v), at least or at least about 25% (v/v), at least or at least about 30% (v/v), at least or at least about 35% (v/v), at least or at least about 40% (v/v), or at least or at least about 50%. In some embodiments, the sample contains serum or plasma at a concentration that is or is approximately about or at least about 25%, 26%, 27%, 28%, 29%, 30%, 31%, 32%, 33%, 34%, or 35% (v/v). In some embodiments, the serum or plasma is human. In some embodiments, the serum or plasma is autologous to the subject. In some embodiments, prior to the selection and/or transduction of cells, the sample containing primary cells is contacted with or contains an anticoagulant. In some embodiments, the anti-coagulant is or contains free citrate ion, e.g. anticoagulant citrate dextrose solution, Solution A (ACD-A).

In some embodiments, prior to the selection and/or transduction of cells, the sample is maintained at a temperature of from or from about 2° C. to 8° C. for up to 48 hours, such as for up to 12 hours, 24 hours or 36 hours.

In some embodiments, the input composition comprises and/or is enriched for T cells, including CD4+ and/or CD8+ T cells. In some aspects, enrichment can be carried out by affinity-based selection by incubation of primary cells with one or more selection or affinity reagent that specifically binds to a cell surface molecule expressed on a subpopulation of the primary cells, thereby enriching the primary cells based on binding to the selection reagent. In some embodiments, enrichment can be carried out by incubation of cells with antibody-coated particles, e.g., magnetic beads.

In some embodiments, the input composition comprises greater than or greater than about 75%, 80%, 85%, 90%, 95% or more T cell obtained from a sample from a subject. In some aspects, prior to the incubation, no more than 5%, 10%, 20%, 30%, or 40% of the T cells of the input composition are activated cells, express a surface marker selected from the group consisting of HLA-DR, CD25, CD69, CD71, CD40L and 4-1BB; comprise intracellular expression of a cytokine selected from the group consisting of IL-2, IFN-gamma, TNF-alpha, are in the G1 or later phase of the cell cycle, and/or are capable of proliferating.

In some embodiments, an input composition containing such cells, e.g. cells that have not been subjected to ex vivo stimulation with a stimulating agent or agents prior to the incubating and/or contacting, is one in which greater than greater than 20%, 30%, 40%, 50%, 60%, 70% or more of the cells express the low-density lipid receptor (LDL-R). In some embodiments, the input composition is enriched and/or selected for T cells, such as CD4+ and/or CD8+ T cells, and, prior to said incubating, greater than 20%, 30%, 40%, 50%, 60%, 70% or more of the T cells express the low-density lipid receptor (LDL-R).

In some embodiments, during or during at least a portion of the incubating and/or contacting, the input composition can comprise one or more cytokines. In some embodiments, the cytokine is selected from IL-2, IL-7 or IL-15. In some embodiments, the cytokine is a recombinant cytokine. In some embodiments, the concentration of the cytokine in the input composition, independently, is from or from about 1 IU/mL to 1500 IU/mL, such as from or from about 1 IU/mL to 100 IU/mL, 2 IU/mL to 50 IU/mL, 5 IU/mL to 10 IU/mL, 10 IU/mL to 500 IU/mL, 50 IU/mL to 250 IU/mL or 100 IU/mL to 200 IU/mL, 50 IU/mL to 1500 IU/mL, 100 IU/mL to 1000 IU/mL or 200 IU/mL to 600 IU/mL. In some embodiments, the concentration of the cytokine in the input composition, independently, is at least or at least about 1 IU/mL, 5 IU/mL, 10 IU/mL, 50 IU/mL, 100 IU/mL, 200 IU/mL, 500 IU/mL, 1000 IU/mL or 1500 IU/mL. In certain aspects, an agent capable of activating an intracellular signaling domain of a TCR complex, such as an anti-CD3 and/or anti-CD28 antibody, also can be including during or during at least a portion of the incubating or subsequent to the incubating.

In some embodiments, during or during at least a portion of the incubating and/or contacting, the input composition can comprises serum. In some embodiments, the serum is fetal bovine serum. In some embodiments, the serum is human serum. In some embodiments, the serum is present in the input composition at a concentration from or from about 0.5% to 25% (v/v), 1.0% to 10% (v/v) or 2.5% to 5.0% (v/v), each inclusive. In some embodiments, the serum is present in the input composition at a concentration that is at least or at least about 0.5% (v/v), 1.0% (v/v), 2.5% (v/v), 5% (v/v) or 10% (v/v).

In some embodiments, during or during at least a portion of the incubating and/or contacting, the input composition is free and/or substantially free of serum. In some embodiments, during or during at least a portion of the incubating and/or contacting, the input composition is incubated and/or contacted in the absence of serum. In particular embodiments, during or during at least a portion of the incubating and/or contacting, the input composition is incubated and/or contacted in serum-free media. In some embodiments, the serum free media is a defined and/or well-defined cell culture media. In certain embodiments, the serum free media is a controlled culture media that has been processed, e.g., filtered to remove inhibitors and/or growth factors. In some embodiments, the serum free media contains proteins. In certain embodiments, the serum-free media may contain serum albumin, hydrolysates, growth factors, hormones, carrier proteins, and/or attachment factors. In some embodiments, the serum-free media contains proteins, e.g., albumin, such as bovine serum albumin, human serum albumin, and/or recombinant albumin. In some embodiments, the serum free media contains a basal media, e.g., DMEM or RPMI 1640, containing amino acids, vitamins, inorganic salts, buffers, antioxidants and energy sources. In some embodiments, the serum free media is supplemented, such as with, but not limited to, albumin, chemically defined lipids, growth factors, insulin, cytokines, and/or antioxidants. In some embodiments, the serum free media is formulated to support growth, proliferation, health, homeostasis of cells of a certain cell type, such as immune cells, T cells, and/or CD4+ and CD8+ T cells.

In some embodiments, during or during at least a portion of the incubating and/or contacting, the input composition comprises N-Acetylcysteine. In some embodiments, the concentration of N-Acetylcysteine in the input composition is from or from about 0.4 mg/mL to 4 mg/mL, 0.8 mg/mL to 3.6 mg/mL or 1.6 mg/mL to 2.4 mg/mL, each inclusive. In some embodiments, the concentration of N-Acetylcysteine in the input composition is at least or at least about or about 0.4 mg/mL, 0.8 mg/mL, 1.2 mg/mL, 1.6 mg/mL, 2.0 mg/mL, 2.4 mg/mL, 2.8 mg/mL, 3.2 mg/mL, 3.6 mg/mL or 4.0 mg/mL.

In some embodiments, the concentration of cells of the input composition is from or from about 1.0×10⁵ cells/mL to 1.0×10⁸ cells/mL, such as at least or about at least or about 1.0×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL.

In some embodiments, the viral particles are provided at a certain ratio of copies of the viral vector particles or infectious units (IU) thereof, per total number of cells (IU/cell) in the input composition or total number of cells to be transduced. For example, in some embodiments, the viral particles are present during the contacting at or about or at least at or about 0.5, 1, 2, 3, 4, 5, 10, 15, 20, 30, 40, 50, or 60 IU of the viral vector particles per one of the cells.

In some embodiments, the titer of viral vector particles is between or between about 1×10⁶ IU/mL and 1×10⁸ IU/mL, such as between or between about 5×10⁶ IU/mL and 5×10⁷ IU/mL, such as at least 6×10⁶ IU/mL, 7×10⁶ IU/mL, 8×10⁶ IU/mL, 9×10⁶ IU/mL, 1×10⁷ IU/mL, 2×10⁷ IU/mL, 3×10⁷ IU/mL, 4×10⁷ IU/mL, or 5×10⁷ IU/mL.

In some embodiments, transduction can be achieved at a multiplicity of infection (MOI) of less than 100, such as generally less than 60, 50, 40, 30, 20, 10, 5 or less.

In some embodiments, the method involves contacting or incubating, such as admixing, the cells with the viral particles. In some embodiments, the contacting is for 30 minutes to 72 hours, such as 30 minute to 48 hours, 30 minutes to 24 hours or 1 hour to 24 hours, such as at least or about at least 30 minutes, 1 hour, 2 hours, 6 hours, 12 hours, 24 hours, 36 hours or more.

In some embodiments, contacting is performed in solution. In some embodiments, the cells and viral particles are contacted in a volume of from or from about 0.5 mL to 500 mL, such as from or from about 0.5 mL to 200 mL, 0.5 mL to 100 mL, 0.5 mL to 50 mL, 0.5 mL to 10 mL, 0.5 mL to 5 mL, 5 mL to 500 mL, 5 mL to 200 mL, 5 mL to 100 mL, 5 mL to 50 mL, 5 mL to 10 mL, 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL.

In some embodiments, when the contacting can be effected with centrifugation, such as spinoculation (e.g. centrifugal inoculation). In some embodiments, the composition containing cells, viral particles and reagent can be rotated, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm). In some embodiments, the rotation is carried at a force, e.g., a relative centrifugal force, of from or from about 100 g to 3200 g (e.g. at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, the incubation of the cells with the viral vector particles results in or produces an output composition comprising cells transduced with the viral vector particles.

D. Other Processing Steps

In some embodiments, the processing steps for transduction, such as in connection with cell engineering, can additionally include culture, cultivation, stimulation, activation, propagation and/or formulation of cells. In some embodiments, the output compositions or cells are incubated in the presence of stimulating conditions or a stimulatory agent. Such conditions include those designed to induce proliferation, expansion, activation, and/or survival of cells in the population and/or to mimic antigen exposure. The stimulation can be carried out ex vivo or in vivo after administration to the subject.

1. Post-Transduction Activation and/or Expansion of Cells

In some embodiments, the cells, e.g. output composition, are further incubated and/or further cultured in connection with genetic engineering. The incubation steps can include culture, cultivation, stimulation, activation, and/or propagation. In some such embodiments, the further incubation is effected under conditions to result in integration of the viral vector into a host genome of one or more of the cells. The incubation and/or engineering may be carried out in a culture vessel, such as a unit, chamber, well, column, tube, tubing set, valve, vial, culture dish, bag, or other container for culture or cultivating cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

In some embodiments, the further incubation is carried out at temperatures greater than room temperature, such as greater than or greater than about 25° C., such as generally greater than or greater than about 32° C., 35° C. or 37° C. In some embodiments, the further incubation is effected at a temperature of at or about 37° C.±2° C., such as at a temperature of at or about 37° C.

In some embodiments, the further incubation is performed under conditions for stimulation and/or activation of cells, which conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent (e.g. stimulatory and/or accessory agents), e.g., ligand, which is capable of activating an intracellular signaling domain of a TCR complex. In some aspects, the agent turns on or initiates TCR/CD3 intracellular signaling cascade in a T cell, such as agents suitable to deliver a primary signal, e.g., to initiate activation of an ITAM-induced signal, such as those specific for a TCR component, and/or an agent that promotes a costimulatory signal, such as one specific for a T cell costimulatory receptor, e.g., anti-CD3, anti-CD28, or anti-41-BB, for example, optionally bound to solid support such as a bead, and/or one or more cytokines. Among the stimulating agents are anti-CD3/anti-CD28 beads (e.g., DYNABEADS® M-450 CD3/CD28 T Cell Expander, and/or ExpACT® beads). Optionally, the expansion method may further comprise the step of adding anti-CD3 and/or anti-CD28 antibody to the culture medium. In some embodiments, the stimulating agents include IL-2 and/or IL-15, for example, an IL-2 concentration of at least about 10 units/mL.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2 and/or IL-15, for example, an IL-2 concentration of at least about 10 units/mL, at least about 50 units/mL, at least about 100 units/mL or at least about 200 units/mL.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the further incubation is carried out in the same container or apparatus in which the contacting occurred. In some embodiments, the further incubation is carried out without rotation or centrifugation, which generally is carried out subsequent to the at least portion of the incubation done under rotation, e.g. in connection with centrifugation or spinoculation. In some embodiments, the further incubation is carried out outside of a stationary phase, such as outside of a chromatography matrix, for example, in solution.

In some embodiments, the further incubation is carried out in a different container or apparatus from that in which the contacting occurred, such as by transfer, e.g. automatic transfer, of the cell composition into a different container or apparatus subsequent to contacting with the viral particles and reagent.

In some embodiments, the T cells are expanded by adding to a culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing virus-transformed lymphoblastoid cells (LCL) as feeder cells, for example, the virus may be EBV, CMV, or influenza and the transformed LCLs present virally-derived antigen on their surface, optionally in the context of an MHC. In some embodiments, the T cells express a TCR that recognizes a viral antigen. In some embodiments, the viral antigen can be from EBV, CMV, or influenza. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In some embodiments, the further culturing or incubation, e.g. to facilitate ex vivo expansion, is carried out of for greater than or greater than about 24 hours, 2 days, 3 days, 4 days, 5 days, 6 days, 7 days, 8 days, 9 days, 10 days, 11 days, 12 days, 13 days or 14 days. In some embodiments, the further culturing or incubation is carried out for no more than 6 days, no more than 5 days, no more than 4 days, no more than 3 days, no more than 2 days or no more than 24 hours.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

In some embodiments, the methods provided herein do not include further culturing or incubation, e.g. do not include ex vivo expansion step, or include a substantially shorter ex vivo expansion step.

In some embodiments, the entire process of engineering the cells, e.g. selection and/or enrichment, incubation in connection with transduction and/or further culturing or cultivation is carried out within a time period of more than 9 days, no more than 8 days, no more than 7 days, no more than 6 days, no more than 5 days, no more than 4 days, no more than 3 days, no more than 2 days or no more than 1 days following obtaining a sample from a subject. It is understood that such timing does not include any period of time in which the cells are subjected to cryopreservation.

In some embodiments of the methods provided herein, engineered cells, e.g. output composition or formulated composition, are administered to the subject immediately or shortly after transduction, without significant ex vivo expansion. In some embodiments, the engineered cells can be administered immediately after the transduction step. In some embodiments, the engineered cells can be administered shortly after the transduction step, e.g., with no significant ex vivo expansion or substantially shorter ex vivo expansion than in conventional methods, which can require significant in vitro activation, expansion and/or enrichment. For example, in some embodiments of the methods provided herein, the engineered cells can be administered within three, two or one day of transduction. In some embodiments, the engineered cells can be administered within 48, 36, 24, 20, 16, 12, 8, 4, 2, 1 or fewer hours of the transduction step. In some embodiments, the engineered cells are subject to a substantially shorter in vitro expansion than conventional methods, e.g., for 48, 36, 24, 20, 16, 12, 8, 4, 2, 1 or fewer hours.

In any of such embodiments, expansion and/or activation of cells can occur in vivo after exposure to antigen, e.g., expansion of the engineered cells in the body of the subject after administration of the cells. In some embodiments, the extent, degree or magnitude of in vivo expansion can be further augmented, boosted or enhanced by various methods that are able to modulate, e.g. increase, expansion, proliferation, survival and/or efficacy of the administered cells, e.g., recombinant receptor expressing cells.

In some embodiments, such methods include those involving administration of engineered cells that are further modified with an agent, e.g. nucleic acid, to alter (e.g. increase or decrease) expression or activity of a molecule, in which such altered expression or activity augments, boosts or enhances the expansion, proliferation, survival and/or efficacy of the administered cells. In some embodiments, the expression of the agent, e.g. a nucleic acid, is inducible, repressible, regulatable and/or user controlled, such as by administration of an inducer or other modulating molecule.

In some embodiments, such methods include methods involving the combined administration, e.g. simultaneous or sequential administration, with a drug or agent capable of augmenting, boosting or enhancing the expansion, proliferation, survival and/or efficacy of the administered cells, e.g., recombinant receptor expressing cells. Exemplary of such drugs and agents are described in Section III.

2. Formulating

In some embodiments, subsequent to the further incubation, the process for preparing the cells can further include washing or formulating the cells. Thus, among the processing steps may include formulating such compositions.

Also provided are pharmaceutical compositions or formulations for use in such methods, which in some embodiments are formulated in connection with the provided processing methods, such as in the closed system in which other processing steps are carried out, such as in an automated or partially automated fashion.

In some embodiments, the cells and compositions are administered to a subject in the form of a pharmaceutical composition or formulation, such as a composition comprising the cells or cell populations and a pharmaceutically acceptable carrier or excipient.

The term “pharmaceutical formulation” refers to a preparation which is in such form as to permit the biological activity of an active ingredient contained therein to be effective, and which contains no additional components which are unacceptably toxic to a subject to which the formulation would be administered.

The pharmaceutical compositions in some embodiments additionally comprise other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

### III. Therapeutic Methods and Compositions for Administration

In some aspects, the products of the methods are used in methods of treatment, e.g. therapeutic methods, such as for administrating the cells and compositions to subjects in adoptive cell therapy. Also provided are such methods and uses of cells processed and produced by the methods, and pharmaceutical compositions and formulations for use therein. The provided methods generally involve administering the cells or compositions, e.g., output composition and/or formulated compositions, to subjects.

In some embodiments, the cells express recombinant receptors, such as CARs, or other antigen receptors, such as transgenic TCRs, e.g., those transferred in the transduction methods provided herein. Such cells generally are administered to subjects having a disease or condition that is associated with a ligand specifically recognized by the receptor. In one embodiment, the cells express a recombinant receptor or a chimeric receptor, such as an antigen receptor, e.g. a CAR or a TCR, that specifically binds to a ligand associated with the disease or condition or expressed by a cell or tissue thereof. For example, in some embodiments, the receptor is an antigen receptor and the ligand is an antigen specific for and/or associated with the disease or condition. The administration generally effects an improvement in one or more symptoms of the disease or condition and/or treats or prevents the disease or condition or a symptom thereof.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, and parasitic disease, and autoimmune and inflammatory diseases. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), ALL, non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

In some embodiments, such diseases include but are not limited to limited to leukemia, lymphoma, e.g., acute myeloid (or myelogenous) leukemia (AML), chronic myeloid (or myelogenous) leukemia (CML), acute lymphocytic (or lymphoblastic) leukemia (ALL), chronic lymphocytic leukemia (CLL), hairy cell leukemia (HCL), small lymphocytic lymphoma (SLL), Mantle cell lymphoma (MCL), Marginal zone lymphoma, Burkitt lymphoma, Hodgkin lymphoma (HL), non-Hodgkin lymphoma (NHL), Anaplastic large cell lymphoma (ALCL), follicular lymphoma, refractory follicular lymphoma, diffuse large B-cell lymphoma (DLBCL) and multiple myeloma (MM). In some embodiments, disease or condition is a B cell malignancy selected from among acute lymphoblastic leukemia (ALL), adult ALL, chronic lymphoblastic leukemia (CLL), non-Hodgkin lymphoma (NHL), and Diffuse Large B-Cell Lymphoma (DLBCL). In some embodiments, the disease or condition is NHL and the NHL is selected from the group consisting of aggressive NHL, diffuse large B cell lymphoma (DLBCL), NOS (de novo and transformed from indolent), primary mediastinal large B cell lymphoma (PMBCL), T cell/histocyte-rich large B cell lymphoma (TCHRBCL), Burkitt's lymphoma, mantle cell lymphoma (MCL), and/or follicular lymphoma (FL), optionally, follicular lymphoma Grade 3B (FL3B).

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder or includes αvβ6 integrin (avb6 integrin), B cell maturation antigen (BCMA), B7-H3, B7-H6, carbonic anhydrase 9 (CA9, also known as CAIX or G250), a cancer-testis antigen, cancer/testis antigen _{1B} (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRL5; also known as Fc receptor homolog 5 or FCRH5), fetal acetylcholine receptor (fetal AchR), a folate binding protein (FBP), folate receptor alpha, ganglioside GD2, O-acetylated GD2 (OGD2), ganglioside GD3, glycoprotein 100 (gp100), G Protein Coupled Receptor 5D (GPCR5D), Her2/neu (receptor tyrosine kinase erb-B2), Her3 (erb-B3), Her4 (erb-B4), erbB dimers, Human high molecular weight-melanoma-associated antigen (HMW-MAA), hepatitis B surface antigen, Human leukocyte antigen A1 (HLA-A), Human leukocyte antigen A2 (HLA-A2), IL-22 receptor alpha(IL-22Ra), IL-13 receptor alpha 2 (IL-13Ra2), kinase insert domain receptor (kdr), kappa light chain, L cell adhesion molecule (L-CAM), CE7 epitope of L1-CAM, Leucine Rich Repeat Containing 8 Family Member A (LRRC8A), Lewis Y, Melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, Preferentially expressed antigen of melanoma (PRAME), progesterone receptor, a prostate specific antigen, prostate stem cell antigen (PSCA), prostate specific membrane antigen (PSMA), Receptor Tyrosine Kinase Like Orphan Receptor 1 (ROR1), survivin, Trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms Tumor 1 (WT-1), a pathogen-specific antigen, or an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen is or includes CD20, CD19, CD22, ROR1, CD45, CD21, CD5, CD33, Igkappa, Iglambda, CD79a, CD79b or CD30.

In some embodiments, antigen associated with the disease or disorder that is targeted by the cells or compositions is selected from the group consisting of orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acetylcholine receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens.

In some embodiments, the antigen is or includes a pathogen-specific or pathogen-expressed antigen. In some embodiments, the antigen is a viral antigen (such as a viral antigen from HIV, HCV, HBV, etc.), bacterial antigens, and/or parasitic antigens.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

The disease or condition that is treated can be any in which expression of an antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by a bacterial, viral or other pathogen. Exemplary antigens, which include antigens associated with various diseases and conditions that can be treated, are described above. In particular embodiments, the chimeric antigen receptor or transgenic TCR specifically binds to an antigen associated with the disease or condition.

The cells and compositions may be administered using standard administration techniques, formulations, and/or devices. Administration of the cells can be autologous or heterologous, such as allogeneic. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

In some embodiments, cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, and following isolation and processing the cells are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells, by multiple bolus administrations of the cells for example, over a period of no more than 3 days, or by continuous infusion administration of the cells.

A. Dosages and Administration

The cells or compositions are administered at a dose to result in a therapeutically effective amount of recombinant receptor (e.g. CAR)-expressing cells in vivo for treating the disease or condition. For the prevention or treatment of disease, the appropriate dosage may depend on the type of disease to be treated, the type of cells or recombinant receptors, administration of other drugs or agents in combination, such as those that boost, augment or enhance cell expansion, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered and other drugs or agents being administered in combination, such as concurrently.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

In some embodiments, the cells or compositions are administered in an amount that is effective to treat or prevent the disease or condition, such as a therapeutically effective or prophylactically effective amount. Thus, in some embodiments, the methods of administration include administration of the cells and compositions at effective amounts. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined.

In some embodiments, a therapeutically effective amount of cells are administered to the subject. In some embodiments, a sub-optimal dose of cells are administered to a subject, such as in certain cases in which the cells are administered under conditions for in vivo expansion of cells.

In certain embodiments, the cells, or individual populations of sub-types of cells, are administered to the subject at a range of about 0.1 million to about 100 billion cells and/or that amount of cells per kilogram of body weight of the subject, such as, e.g., 0.1 million to about 50 billion cells (e.g., less than 0.5 million cells, less than 1 million cells, about 0.1 million cells, about 0.2 million cells, about 0.3 million cells, about 0.4 million cells, about 0.5 million cells, about 1 million cells, about 5 million cells, about 25 million cells, about 500 million cells, about 1 billion cells, about 5 billion cells, about 20 billion cells, about 30 billion cells, about 40 billion cells, or a range defined by any two of the foregoing values), 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight of the subject. Dosages may vary depending on attributes particular to the disease or disorder and/or patient and/or other treatments. In some embodiments, such values refer to numbers of recombinant receptor-expressing cells; in other embodiments, they refer to number of T cells or PBMCs or total cells administered.

In some embodiments, the cell therapy comprises administration of a dose comprising a number of cells that is at least or at least about or is or is about 0.1×10⁶ cells/kg body weight of the subject, 0.2×10⁶ cells/kg, 0.3×10⁶ cells/kg, 0.4×10⁶ cells/kg, 0.5×10⁶ cells/kg, 1×10⁶ cell/kg, 2.0×10⁶ cells/kg, 3×10⁶ cells/kg or 5×10⁶ cells/kg.

In some embodiments, the cell therapy comprises administration of a dose comprising a number of cells is between or between about 0.1×10⁶ cells/kg body weight of the subject and 1.0×10⁷ cells/kg, between or between about 0.5×10⁶ cells/kg and 5×10⁶ cells/kg, between or between about 0.5×10⁶ cells/kg and 3×10⁶ cells/kg, between or between about 0.5×10⁶ cells/kg and 2×10⁶ cells/kg, between or between about 0.5×10⁶ cells/kg and 1×10⁶ cell/kg, between or between about 1.0×10⁶ cells/kg body weight of the subject and 5×10⁶ cells/kg, between or between about 1.0×10⁶ cells/kg and 3×10⁶ cells/kg, between or between about 1.0×10⁶ cells/kg and 2×10⁶ cells/kg, between or between about 2.0×10⁶ cells/kg body weight of the subject and 5×10⁶ cells/kg, between or between about 2.0×10⁶ cells/kg and 3×10⁶ cells/kg, or between or between about 3.0×10⁶ cells/kg body weight of the subject and 5×10⁶ cells/kg, each inclusive.

In some embodiments, the dose of cells comprises between at or about 2×10⁵ of the cells/kg and at or about 2×10⁶ of the cells/kg, such as between at or about 4×10⁵ of the cells/kg and at or about 1×10⁶ of the cells/kg or between at or about 6×10⁵ of the cells/kg and at or about 8×10⁵ of the cells/kg. In some embodiments, the dose of cells comprises no more than 2×10⁵ of the cells (e.g. antigen-expressing, such as CAR-expressing cells) per kilogram body weight of the subject (cells/kg), such as no more than at or about 3×10⁵ cells/kg, no more than at or about 4×10⁵ cells/kg, no more than at or about 5×10⁵ cells/kg, no more than at or about 6×10⁵ cells/kg, no more than at or about 7×10⁵ cells/kg, no more than at or about 8×10⁵ cells/kg, no more than at or about 9×10⁵ cells/kg, no more than at or about 1×10⁶ cells/kg, or no more than at or about 2×10⁶ cells/kg.

In some embodiments, the dose of cells comprises at least or at least about or at or about 2×10⁵ of the cells (e.g. antigen-expressing, such as CAR-expressing cells) per kilogram body weight of the subject (cells/kg), such as at least or at least about or at or about 3×10⁵ cells/kg, at least or at least about or at or about 4×10⁵ cells/kg, at least or at least about or at or about 5×10⁵ cells/kg, at least or at least about or at or about 6×10⁵ cells/kg, at least or at least about or at or about 7×10⁵ cells/kg, at least or at least about or at or about 8×10⁵ cells/kg, at least or at least about or at or about 9×10⁵ cells/kg, at least or at least about or at or about 1×10⁶ cells/kg, or at least or at least about or at or about 2×10⁶ cells/kg.

In some embodiments, the dose of cells is a flat dose of cells or fixed dose of cells such that the dose of cells is not tied to or based on the body surface area or weight of a subject.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 5×10⁸ total recombinant receptor (e.g., CAR)-expressing cells, T cells, or peripheral blood mononuclear cells (PBMCs), e.g., in the range of about 1×10⁶ to 5×10⁸ such cells, such as 2×10⁶, 5×10⁶, 1×10⁷, 5×10⁷, 1×10⁸, or 5×10⁸ total such cells, or the range between any two of the foregoing values.

In some embodiments, the cell therapy comprises administration of a dose comprising a number of cell from or from about 1×10⁵ to 5×10⁸ total recombinant receptor-expressing cells, total T cells, or total peripheral blood mononuclear cells (PBMCs), from or from about 5×10⁵ to 1×10⁷ total recombinant receptor-expressing cells, total T cells, or total peripheral blood mononuclear cells (PBMCs) or from or from about 1×10⁶ to 1×10⁷ total recombinant receptor-expressing cells, total T cells, or total peripheral blood mononuclear cells (PBMCs), each inclusive. In some embodiments, the cell therapy comprises administration of a dose of cells comprising a number of cells at least or at least about 1×10⁵ total recombinant receptor-expressing cells, total T cells, or total peripheral blood mononuclear cells (PBMCs), such at least or at least 1×10⁶, at least or at least about 1×10⁷, at least or at least about 1×10⁸ of such cells. In some embodiments, the number is with reference to the total number of CD3+ or CD8+, in some cases also recombinant receptor-expressing (e.g. CAR+) cells. In some embodiments, the cell therapy comprises administration of a dose comprising a number of cell from or from about 1×10⁵ to 5×10⁸ CD3+ or CD8+ total T cells or CD3+ or CD8+ recombinant receptor-expressing cells, from or from about 5×10⁵ to 1×10⁷ CD3+ or CD8+ total T cells or CD3+ or CD8+ recombinant receptor-expressing cells, or from or from about 1×10⁶ to 1×10⁷ CD3+ or CD8+ total T cells or CD3+ or CD8+ recombinant receptor-expressing cells, each inclusive. In some embodiments, the cell therapy comprises administration of a dose comprising a number of cell from or from about 1×10⁵ to 5×10⁸ total CD3+/CAR+ or CD8+/CAR+ cells, from or from about 5×10⁵ to 1×10⁷ total CD3+/CAR+ or CD8+/CAR+ cells, or from or from about 1×10⁶ to 1×10⁷ total CD3+/CAR+ or CD8+/CAR+ cells, each inclusive.

In some embodiments, the T cells of the dose include CD4+ T cells, CD8+ T cells or CD4+ and CD8+ T cells.

In some embodiments, for example, where the subject is human, the CD8+ T cells of the dose, including in a dose including CD4+ and CD8+ T cells, includes between about 1×10⁶ and 5×10⁸ total recombinant receptor (e.g., CAR)-expressing CD8+ cells, e.g., in the range of about 5×10⁶ to 1×10⁸ such cells, such cells 1×10⁷, 2.5×10⁷, 5×10⁷, 7.5×10⁷, 1×10⁸, or 5×10⁸ total such cells, or the range between any two of the foregoing values. In some embodiments, the patient is administered multiple doses, and each of the doses or the total dose can be within any of the foregoing values. In some embodiments, the dose of cells comprises the administration of from or from about 1×10⁷ to 0.75×10⁸ total recombinant receptor-expressing CD8+ T cells, 1×10⁷ to 2.5×10⁷ total recombinant receptor-expressing CD8+ T cells, from or from about 1×10⁷ to 0.75×10⁸ total recombinant receptor-expressing CD8+ T cells, each inclusive. In some embodiments, the dose of cells comprises the administration of or about 1×10⁷, 2.5×10⁷, 5×10⁷ 7.5×10⁷, 1×10⁸, or 5×10⁸ total recombinant receptor-expressing CD8+ T cells.

In some embodiments, the dose of cells, e.g., recombinant receptor-expressing T cells, is administered to the subject as a single dose or is administered only one time within a period of two weeks, one month, three months, six months, 1 year or more.

In the context of adoptive cell therapy, administration of a given “dose” of cells encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as over no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells of the dose are administered in a single pharmaceutical composition. In some embodiments, the cells of the dose are administered in a plurality of compositions, collectively containing the cells of the dose.

The term “split dose” refers to a dose that is split so that it is administered over more than one day. This type of dosing is encompassed by the present methods and is considered to be a single dose. In some embodiments, the cells of a split dose are administered in a plurality of compositions, collectively comprising the cells of the dose, over a period of no more than three days.

Thus, the dose of cells may be administered as a split dose, e.g., a split dose administered over time. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time. For example, the populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells.

In some embodiments, the administration of the composition or dose, e.g., administration of the plurality of cell compositions, involves administration of the cell compositions separately. In some aspects, the separate administrations are carried out simultaneously, or sequentially, in any order. In some embodiments, the dose comprises a first composition and a second composition, and the first composition and second composition are administered 0 to 12 hours apart, 0 to 6 hours apart or 0 to 2 hours apart. In some embodiments, the initiation of administration of the first composition and the initiation of administration of the second composition are carried out no more than 2 hours, no more than 1 hour, or no more than 30 minutes apart, no more than 15 minutes, no more than 10 minutes or no more than 5 minutes apart. In some embodiments, the initiation and/or completion of administration of the first composition and the completion and/or initiation of administration of the second composition are carried out no more than 2 hours, no more than 1 hour, or no more than 30 minutes apart, no more than 15 minutes, no more than 10 minutes or no more than 5 minutes apart.

In some composition, the first composition, e.g., first composition of the dose, comprises CD4+ T cells. In some composition, the first composition, e.g., first composition of the dose, comprises CD8+ T cells. In some embodiments, the first composition is administered prior to the second composition.

In some embodiments, the dose or composition of cells includes a defined or target ratio of CD4+ cells expressing a recombinant receptor to CD8+ cells expressing a recombinant receptor and/or of CD4+ cells to CD8+ cells, which ratio optionally is approximately 1:1 or is between approximately 1:3 and approximately 3:1, such as approximately 1:1. In some aspects, the administration of a composition or dose with the target or desired ratio of different cell populations (such as CD4+:CD8+ ratio or CAR+CD4+:CAR+CD8+ ratio, e.g., 1:1) involves the administration of a cell composition containing one of the populations and then administration of a separate cell composition comprising the other of the populations, where the administration is at or approximately at the target or desired ratio. In some aspects, administration of a dose or composition of cells at a defined ratio leads to improved expansion, persistence and/or antitumor activity of the T cell therapy.

In some embodiments, the subject receives multiple doses, e.g., two or more doses or multiple consecutive doses, of the cells. In some embodiments, two doses are administered to a subject. In some embodiments, the subject receives the consecutive dose, e.g., second dose, is administered approximately 4, 5, 6, 7, 8, 9, 10, 11, 12, 13, 14, 15, 16, 17, 18, 19, 20 or 21 days after the first dose. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the time between the administration of the first dose and the administration of the consecutive dose is about 9 to about 35 days, about 14 to about 28 days, or 15 to 27 days. In some embodiments, the administration of the consecutive dose is at a time point more than about 14 days after and less than about 28 days after the administration of the first dose. In some aspects, the time between the first and consecutive dose is about 21 days. In some embodiments, an additional dose or doses, e.g. consecutive doses, are administered following administration of the consecutive dose. In some aspects, the additional consecutive dose or doses are administered at least about 14 and less than about 28 days following administration of a prior dose. In some embodiments, the additional dose is administered less than about 14 days following the prior dose, for example, 4, 5, 6, 7, 8, 9, 10, 11, 12, or 13 days after the prior dose. In some embodiments, no dose is administered less than about 14 days following the prior dose and/or no dose is administered more than about 28 days after the prior dose.

In some embodiments, the dose of cells, e.g., recombinant receptor-expressing cells, comprises two doses (e.g., a double dose), comprising a first dose of the T cells and a consecutive dose of the T cells, wherein one or both of the first dose and the second dose comprises administration of the split dose of T cells.

In some embodiments, the dose of cells is generally large enough to be effective in reducing disease burden.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9:1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, the methods also include administering one or more additional doses of cells expressing a chimeric antigen receptor (CAR) and/or lymphodepleting therapy, and/or one or more steps of the methods are repeated. In some embodiments, the one or more additional dose is the same as the initial dose. In some embodiments, the one or more additional dose is different from the initial dose, e.g., higher, such as 2-fold, 3-fold, 4-fold, 5-fold, 6-fold, 7-fold, 8-fold, 9-fold or 10-fold or more higher than the initial dose, or lower, such as e.g., higher, such as 2-fold, 3-fold, 4-fold, 5-fold, 6-fold, 7-fold, 8-fold, 9-fold or 10-fold or more lower than the initial dose. In some embodiments, administration of one or more additional doses is determined based on response of the subject to the initial treatment or any prior treatment, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some embodiments, a relatively lower dose of cells, such as a sub-optimal dose of cells or a dose of cells lower than a therapeutically effective amount, may be administered, which, upon in vivo stimulation (e.g. by an endogenous antigen or exogenous agent) can lead to a boost, such as an increase or expansion, in the number of engineered cells present in the subject. In any of such embodiments, expansion and/or activation of cells can occur with exposure to an antigen in vivo, e.g., expansion of the engineered cells in the body of the subject after administration of the cells. In some embodiments, the extent, degree or magnitude of in vivo expansion can be further augmented, boosted or enhanced by various methods that are able to modulate, e.g. increase, expansion, proliferation, survival and/or efficacy of the administered cells, e.g., recombinant receptor expressing cells.

Once the cells are administered to the subject (e.g., human), the biological activity of the cell populations in some aspects is measured by any of a number of known methods. Parameters to assess include specific binding of the cells to antigen, in vivo, e.g., by imaging, or ex vivo, e.g., by ELISA or flow cytometry. In certain embodiments, the ability of the cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

B. Compositions and Formulations

In some embodiments, the dose of cells comprising cells engineered with a recombinant antigen receptor, e.g. CAR or TCR, is provided as a composition or formulation, such as a pharmaceutical composition or formulation. Such compositions can be used in accord with the provided methods, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells or agent. In some embodiments, it is administered by multiple bolus administrations of the cells or agent, for example, over a period of no more than 3 days, or by continuous infusion administration of the cells or agent.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell or an agent that treats or ameliorates symptoms of neurotoxicity), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the agent or cell populations are administered parenterally. The term “parenteral,” as used herein, includes intravenous, intramuscular, subcutaneous, rectal, vaginal, and intraperitoneal administration. In some embodiments, the agent or cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

C. Combination Therapy, e.g. Agents for Modulating Cell Expansion and Activity

In some embodiments, the cells are administered as part of a combination treatment, such as simultaneously with or sequentially with, in any order, another agent, e.g. therapeutic agent, such as a drug. In some embodiments, the other agent, e.g. drug, can be a therapeutic intervention, such as an antibody or engineered cell or receptor or agent, such as a cytotoxic or therapeutic agent. In some embodiments, the other agent, e.g. drug, can be an agent that enhances, augments or boosts the expansion, proliferation, survival and/or efficacy of the cells. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents.

In some embodiments, the methods include methods involving the combined administration, e.g. simultaneous or sequential administration, with a drug or agent capable of augmenting, boosting or enhancing the expansion, proliferation, survival and/or efficacy of the administered cells, e.g., recombinant receptor expressing cells. In some embodiments, such agents are administered simultaneously with or sequentially with, in any order, another agent, such as a drug. In some embodiments, the agent is administered before, during, during the course of or after administration of the cells, e.g., cells expressing a recombinant receptor, e.g. CAR. In some embodiments, such agents include agents that specifically augment, boost or enhance the expansion, proliferation, survival and/or efficacy of the engineered cells by virtue of specifically modulating the transgene, e.g., transgene encoding a recombinant receptor. In some embodiments, such agents include agents that modulate the cell expansion and/or activity of the administered cells, e.g., immune cells, such as T cells.

In some embodiments, the administered cell, e.g., cells engineered to express a recombinant receptor, are modified to augment, boost or enhance the expansion, proliferation, survival and/or efficacy of the administered cells. In some embodiments, the administered cell, e.g., cells engineered to express a recombinant receptor, are modified such that the expansion, proliferation, survival and/or efficacy of the engineered cells can be regulated and/or controlled, such as by administration of an agent.

In some embodiments, the methods include in vivo steps to reduce, inhibit and/or minimize the effects of inhibitory factors that suppress the proliferation, expansion and/or survival of the engineered cells in vivo. In some embodiments, the methods include in vivo steps to promote, support and/or to enhance proliferation, expansion and/or survival of the engineered cells in vivo.

In some embodiments, the additional agent is a small molecule, a peptide, a polypeptide, an antibody or antigen-binding fragment thereof, an antibody mimetic, an aptamer or a nucleic acid molecule (e.g. siRNA), a lipid, a polysaccharide or any combination thereof. In some embodiments, the additional agent is an inhibitor or an activator of a particular factor, molecule, receptor, function and/or enzyme. In some embodiments, the additional agent is an agonist or an antagonist of a particular factor, molecule, receptor, function and/or enzyme. In some embodiments, the additional agent is an analog or a derivative of one or more factors and/or metabolites. In some embodiments, the additional agent is a protein or polypeptide. In some embodiments, the additional agent is a cell, e.g., an engineered cell.

1. Agents for Transgene-Specific Expansion

In some embodiments, the methods include administering agents in addition to the administered cells, e.g., cells engineered to express recombinant receptors, such as in a combination therapy. In some embodiments, the agents that specifically augment, boost or enhance the expansion, proliferation, survival and/or efficacy of the engineered cells by virtue of specifically modulating the transgene, e.g., transgene encoding a recombinant receptor. In some embodiments, the agent specifically targets the transgene, e.g., the recombinant receptor. In some embodiments, the agent specifically binds, activates and/or enhances the activity of the recombinant receptor and/or other functions of all or a part of the recombinant molecule encoded by the transgene. In some embodiments, administration of the agent in combination with the recombinant cells can enhance, boost or augment proliferation, expansion and/or survival of the administered cells, e.g., enhance in vivo expansion of the cells.

In some embodiments, exemplary methods or agents for transgene-specific expansion include endogenous antigen exposure, vaccination, anti-idiotype antibodies or antigen-binding fragment thereof and/or regulatable recombinant receptor. For example, in some embodiments, methods for transgene-specific expansion include vaccination methods. In some embodiments, the agent is a peptide vaccine or a cell-based vaccine, e.g. cells engineered to express a particular antigen recognized by the recombinant receptor (see, e.g., WO 2016/069647, WO 2011/066048, US 2016/0304624, U.S. Pat. No. 9,476,028 and Hailemichael and Overwijk, Int J Biochem Cell Biol. (2014) 53: 46-50). In some embodiments, the methods for transgene-specific expansion include administering anti-idiotype antibodies. Anti-idiotype antibodies, including antigen-binding fragments thereof, specifically recognizes, is specifically targeted to, and/or specifically binds to an idiotope of an antibody or an antigen binding fragment thereof, e.g., the antigen-binding domain of a recombinant receptor such as a chimeric antigen receptor (CAR). An idiotope is any single antigenic determinant or epitope within the variable portion of an antibody. In some embodiments, the anti-idiotype antibodies or antigen-binding fragments thereof are agonists and/or exhibit specific activity to stimulate cells expressing a particular antibody including conjugates or recombinant receptors containing the same or an antigen-binding fragment thereof (see, e.g., U.S. Pat. Publication Nos. US 2016/0096902; US 2016/0068601; US 2014/0322183; US 2015/0175711; US 2015/283178; U.S. Pat. No. 9,102,760; Jena et al. PloS one (2013) 8(3):e57838; Long et al., Nature Medicine (2015) 21(6):581-590; Lee et al., The Lancet (2015) 385(9967):517-528; Zhao et al., PloS One (2014) 9(5):e96697; Leung et al., MAbs. (2015) 7(1):66-76).

2. Agents to Modulate Cell Expansion or Activity

In some embodiments, the methods include modulation of the expansion, proliferation, survival and/or activity of immune cells or immune function in general, including the engineered cells that are administered. In some embodiments, the methods include steps that are generally immunostimulatory or generally promote, enhance, augment and/or boost the expansion, proliferation, survival and/or activity of immune cells, including the administered cells in vivo, e.g., within the body of the subjects. In some embodiments, the agent can reduce, inhibit and/or minimize the effects of inhibitory factors that suppress the proliferation, expansion and/or survival of immune cells, e.g., administered cells, in vivo.

a. Inhibition of Negative Regulators

In some embodiments, the methods include modulating the expansion of the engineered cells, for example, by inhibiting negative regulator of proliferation, expansion and/or activation of administered cells, e.g., engineered immune cells. In particular environment in the body of the subject administered cells expressing the recombinant receptor, can encounter an environment that represses or suppresses the growth, proliferation, expansion and/or survival of the cells, e.g. immunosuppressive environment. For example, immunosuppressive environments can contain immunosuppressive cytokines, regulatory modulators and co-inhibitory receptors. In some embodiments, an additional agent can be used to modulate the expansion of the administered cells, e.g., overcome suppressive environments.

In some embodiments, the additional agent includes an immunomodulatory agent, immune checkpoint inhibitor, modulators of metabolic pathways, adenosine pathway or adenosine receptor antagonist or agonist and modulators of signaling pathways, e.g., kinase inhibitors.

In some embodiments, the additional agent is an immunomodulatory agent, such as an immune checkpoint inhibitor. In some examples, the additional agent increases, enhances or augments the expansion and/or proliferation of the administered cells and thereby increases, enhances or augments the immune response by blocking an immune checkpoint protein (i.e., immune checkpoint inhibitor). In some embodiments, the additional agent is an agent that enhances the activity of the engineered cell, e.g., a recombinant receptor-expressing cell, is a molecule that inhibits an immune inhibitory molecule or an immune checkpoint molecule. Examples of immune inhibitory molecules include PD-1, PD-L1, CTLA4, TEVI3, CEACAM (e.g., CEACAM-1, CEACAM-3 and/or CEACAM-5), LAG3, VISTA, BTLA, TIGIT, LAIR1, CD160, 2B4 and TGFR β. In some embodiments, the immune checkpoint inhibitor can be an antibody directed against an immune checkpoint protein, such as an antibody directed against cytotoxic T-lymphocyte antigen 4 (CTLA4 or CD152), programmed cell death protein 1 (PD-1), or programmed cell death protein 1 ligand 1 (PD-L1) (see, e.g., Pardoll, Nat Rev Cancer. 2012 Mar. 22; 12(4):252-264).

In some embodiments, the methods include contacting the cells expressing the recombinant receptor with an agent that inhibits inhibitory cell surface receptors, e.g., transforming growth factor beta receptor (TGFβR). In some embodiments, administered cells, e.g., recombinant receptor expressing cells, can be engineered to resist the effects of immunosuppressive cytokines that can inhibit their effector functions (see, e.g., Foster et al., J Immunother. (2008) 31:500-505; Bollard et al., Molecular Therapy. (2012) 20:S22; Bendle et al., J. Immunol. (2013) 191(6):3232-3239). In some embodiments, the additional agent is an anti-TGFβ antibody or an anti-TGFβR antibody (see, e.g., WO 2011/109789).

In some embodiments, the additional agent modulates the metabolism, signaling and/or transport of immunosuppressive factors, e.g., adenosine. In some embodiments, the additional agent is an inhibitor of extracellular adenosine or adenosine receptor, or an agent that causes a reduction or a decrease of extracellular adenosine levels, such as an agent that prevents the formation of, degrades, renders inactive, and/or decreases extracellular adenosine. In some embodiments, the additional agent is an adenosine receptor antagonist such as the A2a, A2b and/or A3 receptor.

In some embodiments, the additional agent is a modulator of adenosine levels and/or an adenosine pathway component. Adenosine can function as an immunomodulatory agent in the body. For example, adenosine and some adenosine analogs that non-selectively activate adenosine receptor subtypes decrease neutrophil production of inflammatory oxidative products (Cronstein et al., Ann. N.Y. Acad. Sci. 451:291, 1985; Roberts et al., Biochem. J., 227:669, 1985; Schrier et al., J. Immunol. 137:3284, 1986; Cronstein et al., Clinical Immunol. Immunopath. 42:76, 1987). In some cases, concentration of extracellular adenosine or adenosine analogs can increase in specific environments, e.g., tumor microenvironment (TME). In some cases, adenosine or adenosine analog signaling depends on hypoxia or factors involved in hypoxia or its regulation, e.g., hypoxia inducible factor (HIF). In some embodiments, increase in adenosine signaling can increase in intracellular cAMP and cAMP-dependent protein kinase that results in inhibition of proinflammatory cytokine production, and can lead to the synthesis of immunosuppressive molecules and development of Tregs (Sitkovsky et al., Cancer Immunol Res (2014) 2(7):598-605). In some embodiments, the additional agent can reduce or reverse immunosuppressive effects of adenosine, adenosine analogs and/or adenosine signaling. In some embodiments, the additional agent can reduce or reverse hypoxia-driven A2-adenosinergic T cell immunosuppression. In some embodiments, the additional agent is selected from among antagonists of adenosine receptors, extracellular adenosine-degrading agents, inhibitors of adenosine generation by CD39/CD73 ectoenzymes, and inhibitors of hypoxia-HIF-1α signaling. In some embodiments, the additional agent is an adenosine receptor antagonist or agonist.

Inhibition or reduction of extracellular adenosine or the adenosine receptor by virtue of an inhibitor of extracellular adenosine (such as an agent that prevents the formation of, degrades, renders inactive, and/or decreases extracellular adenosine), and/or an adenosine receptor inhibitor (such as an adenosine receptor antagonist) can enhance immune response, such as a macrophage, neutrophil, granulocyte, dendritic cell, T- and/or B cell-mediated response. In addition, inhibitors of the Gs protein mediated cAMP dependent intracellular pathway and inhibitors of the adenosine receptor-triggered Gi protein mediated intracellular pathways, can also increase acute and chronic inflammation.

In some embodiments, the additional agent is an adenosine receptor antagonist or agonist, e.g., an antagonist or agonist of one or more of the adenosine receptors A2a, A2b, A1, and A3. A1 and A3 inhibit, and A2a and A2b stimulate, respectively, adenylate cyclase activity. Certain adenosine receptors, such as A2a, A2b, and A3, can suppress or reduce the immune response during inflammation. Thus, antagonizing immunosuppressive adenosine receptors can augment, boost or enhance immune response, e.g., immune response from administered cells, e.g., CAR-expressing T cells. In some embodiments, the additional agent inhibits the production of extracellular adenosine and adenosine-triggered signaling through adenosine receptors. For example, enhancement of an immune response, local tissue inflammation, and targeted tissue destruction can be enhanced by inhibiting or reducing the adenosine-producing local tissue hypoxia; by degrading (or rendering inactive) accumulated extracellular adenosine; by preventing or decreasing expression of adenosine receptors on immune cells; and/or by inhibiting/antagonizing signaling by adenosine ligands through adenosine receptors.

An antagonist is any substance that tends to nullify the action of another, as an agent that binds to a cell receptor without eliciting a biological response. In some embodiments, the antagonist is a chemical compound that is an antagonist for an adenosine receptor, such as the A2a, A2b, or A3 receptor. In some embodiments, the antagonist is a peptide, or a pepidomimetic, that binds the adenosine receptor but does not trigger a G1 protein dependent intracellular pathway. Exemplary adenosine receptor antagonists are described in U.S. Pat. Nos. 5,565,566; 5,545,627, 5,981,524; 5,861,405; 6,066,642; 6,326,390; 5,670,501; 6,117,998; 6,232,297; 5,786,360; 5,424,297; 6,313,131, 5,504,090; and 6,322,771; and Jacobson and Gao, Nat Rev Drug Discov. (2006) 5(3): 247-264.

b. Promotion of Immunostimulation

In some embodiments, the methods include administering additional agents that are immunostimulatory. In some embodiments, the additional agent can generally promote the proliferation, expansion, survival and/or efficacy of immune cells. In some embodiments, the additional agent can specifically promote administered cells, e.g., recombinant receptor-expressing cells. In some embodiments, the additional agent is a cytokine. In some embodiments, the additional agent is a ligand.

In some embodiments, the additional agent is an immunostimulatory ligand, e.g., CD40L. In some embodiments, the additional agent is a cytokine, e.g., IL-2, IL-3, IL-6, IL-11, IL-7, IL-12, IL-15, IL-21, granulocyte macrophage colony stimulating factor (GM-CSF), alpha, beta or gamma interferon (IFN) and erythropoietin (EPO).

3. Lymphodepleting Therapies

In some aspects, the provided methods can further include administering one or more lymphodepleting therapies, such as prior to or simultaneous with initiation of administration of the cells, e.g., recombinant receptor-expressing cells. In some embodiments, the lymphodepleting therapy comprises administration of a phosphamide, such as cyclophosphamide. In some embodiments, the lymphodepleting therapy can include administration of fludarabine. In some embodiments, fludarabine is excluded in the lymphodepleting therapy. In some embodiments, a lymphodepleting therapy is not administered.

Preconditioning subjects with immunodepleting (e.g., lymphodepleting) therapies can improve the effects of adoptive cell therapy (ACT). Preconditioning with lymphodepleting agents, including combinations of cyclosporine and fludarabine, have been effective in improving the efficacy of transferred tumor infiltrating lymphocyte (TIL) cells in cell therapy, including to improve response and/or persistence of the transferred cells. See, e.g., Dudley et al., Science, 298, 850-54 (2002); Rosenberg et al., Clin Cancer Res, 17(13):4550-4557 (2011). Likewise, in the context of CAR+ T cells, several studies have incorporated lymphodepleting agents, most commonly cyclophosphamide, fludarabine, bendamustine, or combinations thereof, sometimes accompanied by low-dose irradiation. See Han et al. Journal of Hematology & Oncology, 6:47 (2013); Kochenderfer et al., Blood, 119: 2709-2720 (2012); Kalos et al., Sci Transl Med, 3(95):95ra73 (2011); Clinical Trial Study Record Nos.: NCT02315612; NCT01822652.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the dose of cells. For example, the subject may be administered a preconditioning agent at least 2 days prior, such as at least 3, 4, 5, 6, or 7 days prior, to the first or subsequent dose. In some embodiments, the subject is administered a preconditioning agent no more than 7 days prior, such as no more than 6, 5, 4, 3, or 2 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the cyclophosphamide can be administered in a single dose or can be administered in a plurality of doses, such as given daily, every other day or every three days. In some embodiments, the cyclophosphamide is administered once daily for one or two days. In some embodiments, where the lymphodepleting agent comprises cyclophosphamide, the subject is administered cyclophosphamide at a dose between or between about 100 mg/m² and 500 mg/m², such as between or between about 200 mg/m² and 400 mg/m², or 250 mg/m² and 350 mg/m², inclusive. In some instances, the subject is administered about 300 mg/m² of cyclophosphamide. In some embodiments, the cyclophosphamide can be administered in a single dose or can be administered in a plurality of doses, such as given daily, every other day or every three days. In some embodiments, cyclophosphamide is administered daily, such as for 1-5 days, for example, for 3 to 5 days. In some instances, the subject is administered about 300 mg/m² of cyclophosphamide, daily for 3 days, prior to initiation of the cell therapy.

In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days. In some instances, the subject is administered about 30 mg/m² of fludarabine, daily for 3 days, prior to initiation of the cell therapy. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In one exemplary dosage regime, prior to receiving the first dose, subjects receive a lymphodepleting preconditioning chemotherapy of cyclophosphamide and fludarabine (cy/flu), which is administered at least two days before the first dose of CAR-expressing cells and generally no more than 7 days before administration of cells. After preconditioning treatment, subjects are administered the dose of CAR-expressing T cells as described above.

In some embodiments, the administration of the preconditioning agent prior to infusion of the dose of cells improves an outcome of the treatment. For example, in some aspects, preconditioning improves the efficacy of treatment with the dose or increases the persistence of the recombinant receptor-expressing cells (e.g., CAR-expressing cells, such as CAR-expressing T cells) in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, the administration of the preconditioning agent prior to infusion of the dose of cells improves an outcome of the treatment such as by improving the efficacy of treatment with the dose or increases the persistence of the recombinant receptor-expressing cells (e.g., CAR-expressing cells, such as CAR-expressing T cells) in the subject.

D. Modification of Cells

In certain embodiments, the cells are modified in any number of ways, such that their therapeutic or prophylactic efficacy is increased and/or the expansion, proliferation, survival and/or efficacy can be modulated. In some embodiments, the cells are modified such that the expansion, proliferation, survival and/or efficacy can be modulated, e.g., enhanced, boosted and/or augmented, in vivo after administration to the subject. In some embodiments, the cells are modified such that expression of transgenes and/or immunomodulatory factors can be modulated and/or controlled. In some embodiments, the cells are modified to modulate the expression and/or activity of particular components of the recombinant receptor. In some embodiments, the cells are modified to increase or decrease expression of an agent, e.g. nucleic acid, such as inhibitory nucleic acids. In some embodiments, the cells are modified to express and/or secrete an agent.

In some embodiments, the engineered recombinant receptor, e.g., CAR expressed by the engineered cells can be conjugated either directly or indirectly through a linker to a targeting moiety. The practice of conjugating compounds, e.g., the recombinant receptor, to targeting moieties is known in the art. See, for instance, Wadwa et al., J. Drug Targeting 3: 1 1 1 (1995), and U.S. Pat. No. 5,087,616.

1. Inhibitory Nucleic Acids and Gene Alteration

In some embodiments, the methods include modifying the cells to be administered by contacting the cells with an agent that reduces, or is capable of effecting reduction of, expression of negative regulators of the administered cell, e.g., engineered T cell expressing the recombinant receptor. Negative regulators of the cell include any described herein, such as an immune checkpoint inhibitor, inhibitory receptors and/or adenosine modulators. In some embodiments, the agent that reduces, or is capable of effecting reduction of expression negative regulators includes agents that is or comprises an inhibitory nucleic acid molecule, such as one that is complementary to, targets, inhibits and/or binds a gene or nucleic acid encoding the negative regulator. In some embodiments, the agent is or comprises a complex comprising a ribonucleoprotein (RNP) complex that includes Cas9, e.g. in some cases an enzymatically inactive Cas9, and a gRNA targeting a gene encoding a negative regulator.

In some of any such embodiments, the inhibitory nucleic acid molecule includes an RNA interfering agent. In some of any such embodiments, the inhibitory nucleic acid is or contains or encodes a small interfering RNA (siRNA), a microRNA-adapted shRNA, a short hairpin RNA (shRNA), a hairpin siRNA, a precursor microRNA (pre-miRNA) or a microRNA (miRNA). Methods of designing inhibitory nucleic acids and modifying cells to express inhibitory nucleic acids are known in the art (see, e.g., WO 2004/0455543, and WO 2004/048566).

In some embodiments, the engineered cell is subject to gene alteration, or gene editing, that is targeted to a locus encoding a gene involved in immunomodulation, negative regulation of the immune cell and/or immunosuppression. In some embodiments, gene editing results in an insertion or a deletion at the targeted locus, or a “knock-out” of the targeted locus and elimination of the expression of the encoded protein. In some embodiments, the gene editing is achieved by non-homologous end joining (NHEJ) using a CRISPR/Cas9 system. In some embodiments, one or more guide RNA (gRNA) molecule can be used with one or more Cas9 nuclease, Cas9 nickase, enzymatically inactive Cas9 or variants thereof, or engineered zinc finger or TALE systems. Methods of gene alteration is known in the art (see, e.g., WO 2015/161276; U.S. Pat. Nos. 6,140,081; 6,453,242; and 6,534,261; WO 98/53058; WO 98/53059; WO 98/53060; WO 02/016536, WO 03/016496 and U.S. Publication No. 2011/0301073).

2. Modification to Express Additional Agents

In some embodiments, the cells, e.g., recombinant receptor-expressing cells, are further modified to express and/or secrete the additional agent that promotes, enhances, boosts and/or augments the proliferation, expansion, survival and/or efficacy of the administered cells. For example, the recombinant receptor-expressing cells, e.g., CAR-expressing cells, can be further engineered to express and/or secrete additional agents that overcome immunosuppressive effects and/or enhance expansion and/or function of the T cells and the recombinant receptor. In some embodiments, the cells can be engineered to express cytokines that promote the expansion of the administered cell. In some embodiment, such additional agents can be operatively linked to an inducible expression system, e.g., an inducible promoter.

In some embodiments, the administered cells can be modified to express and/or secrete an agent that inhibits immunosuppressive factors, such as any described herein, and/or stimulates an immunostimulatory factor. In some embodiments, the additional agent expressed by the administered cell, decrease or prevent immunosuppression of said cell in the tumor microenvironment (see, e.g., U.S. Patent Pub. No. US 2016/0045551). In some embodiments, the additional agent encoded and/or secreted by the administered cells can include any of the additional agents described herein.

In some embodiments, the additional agent that is encoded by the administered cell is soluble and is secreted. In some embodiments, the additional agent is a soluble scFv. In some embodiments, the additional agent is a cytokine.

3. Regulation of Expression and/or Activity of Recombinant Receptor

In some embodiments, the methods include modifying the cells to allow regulatable expression and/or activity of the recombinant receptor, e.g., CAR, thereby regulating the signal through the recombinant receptor. In some embodiments, regulatable expression and/or activity is achieved by configuring the recombinant receptor to contain or be controlled by particular regulatory elements and/or systems, such as any described herein. In some embodiments, administration of the engineered cell to the body of the subject and/or exposure to a particular ligand can regulate the expression and/or activity of the recombinant receptor, e.g., CAR. In some embodiments, regulation of expression and/or activity of the recombinant receptor is achieved by administration of an additional agent that can regulate the expression of the recombinant receptor, e.g., CAR. In some embodiments, regulated expression of the recombinant receptor, e.g., CAR, is achieved a regulatable transcription factor release system, or by administration of an additional agent that can induce conformational changes and/or multimerization of polypeptides, e.g., the recombinant receptor. In some embodiments, the additional agent is a chemical inducer.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se.

As used herein, a subject includes any living organism, such as humans and other mammals. Mammals include, but are not limited to, humans, and non-human animals, including farm animals, sport animals, rodents and pets.

As used herein, “depleting” when referring to one or more particular cell type or cell population, refers to decreasing the number or percentage of the cell type or population, e.g., compared to the total number of cells in or volume of the composition, or relative to other cell types, such as by negative selection based on markers expressed by the population or cell, or by positive selection based on a marker not present on the cell population or cell to be depleted. The term does not require complete removal of the cell, cell type, or population from the composition.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” or “+” for a particular marker refers to the detectable presence on or in the cell of a particular marker, typically a surface marker. When referring to a surface marker, the term refers to the presence of surface expression as detected, in some embodiments, by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected, in some embodiments, by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, “percent (%) amino acid sequence identity” and “percent identity” when used with respect to an amino acid sequence (reference polypeptide sequence) is defined as the percentage of amino acid residues in a candidate sequence (e.g., a Vpx or Vpr protein) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

As used herein, “at a position corresponding to” or recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. In some embodiments, exemplary corresponding residues of a Vpx or Vpr protein can be identified by alignment of a sequence with an exemplary Vpx sequence set forth in SEQ ID NO: 1 or other Vpx or Vpr sequence as set forth herein. In some embodiments, exemplary corresponding residues of a SAMHD1 can be identified by alignment of a sequence with an exemplary SAMHD1 sequence set forth in SEQ ID NO: 19. By aligning the sequences, one skilled in the art can identify corresponding residues, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48: 1073).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Vectors include viral vectors, such as retroviral vectors, for example lentiviral or gammaretroviral vectors, having a genome carrying another nucleic acid and capable of inserting into a host genome for propagation thereof.

As used herein, a composition refers to any mixture of two or more products, substances, or compounds, including cells. It may be a solution, a suspension, liquid, powder, a paste, aqueous, non-aqueous or any combination thereof.

As used herein, the terms “treatment,” “treat,” and “treating,” refer to complete or partial amelioration or reduction of a disease or condition or disorder, or a symptom, adverse effect or outcome, or phenotype associated therewith. In certain embodiments, the effect is therapeutic, such that it partially or completely cures a disease or condition or adverse symptom attributable thereto.

As used herein, a “therapeutically effective amount” of a compound or composition or combination refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### V. Exemplary Embodiments

Among the provided embodiments are:

1. A method for transducing T cells, the method comprising incubating a viral vector particle comprising a recombinant nucleic acid and an input composition comprising a plurality of T cells, said plurality of T cells having been obtained from a sample from a subject, wherein:

the incubating is initiated no more than 24 hours after obtaining the sample from the subject; and/or

prior to the incubating, the T cells have not been subjected to a temperature greater than or greater than about 15° C., about 18° C., about 22° C. or about 25° C. for a duration of more than 1 hour, 2 hours, 4 hours, 6 hours, 8 hours, 12 hours, or 24 hours after obtaining the sample from the subject; and/or

prior to the incubating, the T cells have not been subjected to a temperature of, of about, greater than, or greater than about 37°±2.0° C. for a duration of more than 15 minutes, 30 minutes, 1 hour or 2 hours after obtaining the sample from the subject.

2. The method of embodiment 1, wherein the incubating is initiated no more than or no more than about 1 hour, 3 hours, 6 hours, 12 hours or 18 hours after obtaining the sample from the subject.

3. The method of embodiment 1 or embodiment 2, wherein, prior to said incubation, the method does not comprise stimulating the T cells under conditions that promote cell activation.

4. The method of any of embodiments 1-3, wherein, prior to said incubating, the input composition has not been subjected to an ex vivo stimulation comprising incubation greater than or greater than about 37°±2.0° C. and/or incubation in the presence of an agent or agents-capable of activating T cells, CD4+ T cells, and/or CD8+ T cells, incubation in the presence of an agent or agents capable of inducing a signal through a TCR complex and/or incubation in the presence of an agent or agents capable of inducing proliferation of T cells, CD4+ T cells, and/or CD8+ T cells; CD3-binding molecules; CD28-binding molecules; recombinant IL-2; recombinant IL-15; and recombinant IL-7.

5. The method of any of embodiments 1-4, wherein, prior to said incubating, no more than 5%, 10%, 20%, 30%, or 40% of the T cells are activated cells, express a surface marker selected from the group consisting of HLA-DR, CD25, CD69, CD71, CD40L and 4-1BB; comprise intracellular expression of a cytokine selected from the group consisting of IL-2, IFN-gamma, TNF-alpha, are in the G1 or later phase of the cell cycle and/or are capable of proliferating.

6. A method for transducing T cells, the method comprising incubating a viral vector particle comprising a recombinant nucleic acid and an input composition comprising T cells, said T cells having been obtained from a sample from a subject, wherein, prior to said incubating, the T cells or input composition have not been subjected to an ex vivo stimulation comprising incubation greater than or greater than about 37°±2.0° C. and/or incubation in the presence of an agent or agents-capable of activating T cells, CD4+ T cells, and/or CD8+ T cells, incubation in the presence of an agent or agents capable of inducing a signal through a TCR complex and/or incubation in the presence of an agent or agents capable of inducing proliferation of T cells, CD4+ T cells, and/or CD8+ T cells; CD3-binding molecules; CD28-binding molecules; recombinant IL-2; recombinant IL-15; and recombinant IL-7.

7. The method of embodiment 4 or embodiment 6, wherein the one or more agents comprise an anti-CD3 antibody and/or an anti-CD28 antibody.

8. A method for transducing T cells, the method comprising incubating a viral vector particle comprising a recombinant nucleic acid and an input composition comprising T cells, said T cells having been obtained from a sample from a subject, wherein, prior to the incubation, no more than 5%, 10%, 20%, 30%, or 40% of the T cells are activated cells, express a surface marker selected from the group consisting of HLA-DR, CD25, CD69, CD71, CD40L and 4-1BB; comprise intracellular expression of a cytokine selected from the group consisting of IL-2, IFN-gamma, TNF-alpha, and/or are in the G1 or later phase of the cell cycle.

9. The method of embodiment 5 or embodiment 8, wherein no more than 10% of T cells in the input composition comprise a T cell activation marker selected from the group consisting of HLA-DR, CD25, CD69, CD71, CD40L, and 4-1BB immediately prior to the incubation.

10. The method of any of embodiments 1-9, wherein, prior to said incubating, greater than 5%, 10%, 20%, 30%, or 40% of the T cells express the low-density lipid receptor (LDL-R).

11. The method of any of embodiments 1-9, wherein the subject is a human.

12. The method of any of embodiments 1-8, wherein the T cells have not been and/or are not maintained at a temperature of at 2° C. to 8° C. for more than 48 hours prior to the incubating.

13. The method of any of embodiments 1-12, wherein the sample is a blood sample.

14. The method of any of embodiments 1-12, wherein the sample is a leukapheresis sample.

15. The method of embodiment 1-14, wherein the T cells are unfractionated T cells, are enriched or isolated CD3+ T cells, are enriched or isolated CD4+ T cells or are enriched or isolated CD8+ T cells.

16. The method of any of embodiments 1-15, wherein the T cells have been selected or enriched from the sample from the subject.

17. The method of any of embodiments 1-16, further comprising, prior to the incubation, obtaining the sample from the subject and, optionally, selecting or enriching the T cells from the sample, which optionally is an enriched composition and/or generates the input composition.

18. The method of any of embodiments 1-17, wherein the percentage of T cells in the input composition is greater than or greater than about 75%, 80%, 85%, 90%, 95% T cells.

19. The method of any of embodiments 3-18, wherein the T cells comprise CD4+ or CD8+ cells.

20. The method of any of embodiments 3-18, wherein the T cells comprise CD4+ and CD8+ cells.

21. The method of embodiment 20, wherein the ratio of the CD4+ cells to the CD8+ cells is or is about 1:1, 1:2, 2:1, 1:3 or 3:1.

22. The method of any of embodiments 1-21, wherein:

the sample comprises serum or plasma at a concentration of at least or at least about 10% (v/v), at least or at least about 15% (v/v), at least or at least about 20% (v/v), at least or at least about 25% (v/v), at least or at least about 30% (v/v), at least or at least about 33% (v/v), at least or at least about 35% (v/v), or at least or at least about 40% (v/v); and/or

prior to the incubating, the sample has been contacted ex vivo with serum or plasma at a concentration of at least or at least about 10% (v/v), at least or at least about 15% (v/v), at least or at least about 20% (v/v), at least or at least about 25% (v/v), at least or at least about 30% (v/v), at least or at least about 33% (v/v), at least or at least about 35% (v/v), or at least or at least about 40% (v/v).

23. The method of any of embodiments 1-22, wherein:

the sample comprises serum or plasma at a concentration of at least or at least about 30% (v/v); and/or

prior to the incubating, the sample has been contacted ex vivo with serum or plasma at a concentration of at least or at least about 30% (v/v).

24. The method of embodiment 22 or embodiment 23, wherein the serum or plasma is human.

25. The method of any of embodiments 22-24, wherein the serum or plasma is autologous to the subject.

26. The method of any of embodiments 1-22, wherein the sample comprises an anticoagulant.

27. The method of embodiment 26, wherein the anticoagulant comprises free citrate ion.

28. The method of any of embodiments 1-27, wherein, prior to the incubating, the method comprises cryopreserving the T cells, optionally in the sample or the enriched composition, in the presence of a cryoprotectant, thereby producing a cryopreserved composition.

29. The method of embodiment 28, wherein, prior to the incubating, washing the cryopreserved composition under conditions to reduce or remove the cyroprotectant and/or to generate the input composition.

30. The method of any of embodiments 1-29, wherein the input composition comprises N-acetylcysteine (NAC); serum, optionally human serum; recombinant interleukin-2 (IL-2), recombinant interleukin-15 (IL-15), and/or recombinant interleukin-7 (IL-7).

31. The method of any of embodiments 1-30, wherein:

the input composition comprises N-acetylcysteine at a concentration from or from about 0.4 mg/mL to 4 mg/mL, 0.8 mg/mL to 3.6 mg/mL or 1.6 mg/mL to 2.4 mg/mL, each inclusive; or

the input composition comprises N-acetylcysteine at a concentration of at least or at least about or about 0.4 mg/mL, 0.8 mg/mL, 1.2 mg/mL, 1.6 mg/mL, 2.0 mg/mL, 2.4 mg/mL, 2.8 mg/mL, 3.2 mg/mL, 3.6 mg/mL or 4.0 mg/mL.

32. The method of any of embodiments 1-31, wherein:

the input composition comprises serum, optionally human serum, at a concentration from or from about 0.5% to 25% (v/v), 1.0% to 10% (v/v) or 2.5% to 5.0% (v/v), each inclusive; or

the input composition comprises serum, optionally human serum, at a concentration of at least or at least about or about 0.5%, 1.%, 2.5%, 5% (v/v) or 10%.

33. The method of any of embodiments 1-32, wherein:

the input composition comprises recombinant IL-2, optionally recombinant human IL-2, at a concentration from or from about 10 IU/mL to 500 IU/mL, 50 IU/mL to 250 IU/mL or 100 IU/mL to 200 IU/mL, each inclusive; or at a concentration of at least or at least about 10 IU/mL, 50 IU/mL, 100 IU/mL, 200 IU/mL, 300 IU/mL, 400 IU/mL or 500 IU/mL; and/or

the input composition comprises recombinant IL-15, optionally recombinant human IL-15, at a concentration from or from about 1 IU/mL to 100 IU/mL, 2 IU/mL to 50 IU/mL or 5 IU/mL to 10 IU/mL, each inclusive; or at a concentration of at least or at least about 1 IU/mL, 2 IU/mL, 5 IU/mL, 10 IU/mL, 25 IU/mL or 50 IU/mL; and/or

the input composition comprises recombinant IL-7, optionally recombinant human IL-7, at a concentration from or from about 50 IU/mL to 1500 IU/mL, 100 IU/mL to 1000 IU/mL to 200 IU/mL to 600 IU/mL, each inclusive; or at a concentration of at least or at least about 50 IU/mL, 100 IU/mL, 200 IU/mL, 300 IU/mL, 400 IU/mL, 500 IU/mL, 600 IU/mL, 700 IU/mL, 800 IU/mL, 900 IU/mL or 1000 IU/mL.

34. The method of any of embodiments 1-33, wherein the incubating comprises a step of spinoculating the viral vector particles with the input composition.

35. The method of embodiment 34, wherein spinoculating comprises rotating, in an internal cavity of a centrifugal chamber, the viral vector particles and input composition, wherein the rotation is at a relative centrifugal force at an internal surface of the side wall of the cavity that is:

between or between about 500 g and 2500 g, 500 g and 2000 g 500 g and 1600 g, 500 g an 1000 g, 600 g and 1600 g, 600 g and 1000 g, 1000 g and 2000 g or 1000 g and 1600 g, each inclusive; or

at least or at least about 600 g, 800 g, 1000 g, 1200 g, 1600 g, or 2000 g.

36. The method of embodiment 34 or embodiment 35, wherein spinoculating is for a time that is:

greater than or about 5 minutes, greater than or about 10 minutes, greater than or about 15 minutes, greater than or about 20 minutes, greater than or about 30 minutes, greater than or about 45 minutes, greater than or about 60 minutes, greater than or about 90 minutes or greater than or about 120 minutes; or

between or between about 5 minutes and 60 minutes, 10 minutes and 60 minutes, 15 minutes and 60 minutes, 15 minutes and 45 minutes, 30 minutes and 60 minutes or 45 minutes and 60 minutes, each inclusive.

37. The method of any of embodiments 1-36, further comprising contacting the input composition and/or viral vector particles with a transduction adjuvant.

38. The method of embodiment 37, wherein the contacting is carried out prior to, concomitant with or after spinoculating the viral vector particles with the input composition.

39. The method of any of embodiments 1-38, wherein at least a portion of the incubation is carried out at or about 37° C.±2° C.

40. The method of any of embodiments 34-39, wherein the at least a portion of the incubation is carried out after the spinoculation.

41. The method of embodiment 39 or embodiment 40, wherein the at least a portion of the incubation is carried out for no more than or no more than about 2 hours, 4 hours, 12 hours, 18 hours, 24 hours, 30 hours, 36 hours, 48 hours, 60 hours or 72 hours.

42. The method of any of embodiments 39-41, wherein the at least a portion of the incubation is carried out for or for about 24 hours.

43. The method of any of embodiments 1-42, wherein the total duration of the incubation is for no more than 12 hours, 24 hours, 36 hours, 48 hours or 72 hours.

44. The method of any of embodiments 1-43, wherein the viral vector particle is a lentiviral vector particle.

45. The method of embodiment 44, wherein the lentiviral vector particle is derived from HIV-1.

46. The method of any of embodiments 1-45, wherein the viral vector particle is pseudotyped with a viral envelope glycoprotein.

47. The method of embodiment 46, wherein the viral envelope glycoprotein is VSV-G.

48. The method of any of embodiments 1-47, wherein the viral vector particle comprises a lentiviral protein that exhibits SAMHD1-inhibiting activity, said protein being packaged in the viral particle.

49. The method of embodiment 48, wherein the SAMHD1-inhibiting protein is a wild-type Vpx protein, a wild-type Vpr protein, or is a variant or portion of a wild-type Vpx or Vpr protein that exhibits SAMHD1-inhibiting activity.

50. The method of embodiment 48 or embodiment 49, wherein the SAMHD1-inhibiting protein is heterologous to the retroviral vector particle.

51. The method of any of embodiments 48-50, wherein the SAMHD1-inhibiting protein is a wild-type Vpx protein or is a variant or portion of a wild-type Vpx protein that exhibits SAMHD1-inhibiting activity.

52. The method of any of embodiments 1-51, wherein the viral vector particle is incubated at a multiplicity of infection of less than or less than about 20.0 or less than or less than about 10.0.

53. The method of any of embodiments 1-52, wherein:

the viral vector particle is incubated at a multiplicity of infection from or from about 1.0 IU/cell to 10 IU/cell or 2.0 U/cell to 5.0 IU/cell; or

the viral vector particle is incubated at a multiplicity of infection of at least or at least about 1.6 IU/cell, 1.8 IU/cell, 2.0 IU/cell, 2.4 IU/cell, 2.8 IU/cell, 3.2 IU/cell or 3.6 IU/cell, 4.0 IU/cell, 5.0 IU/cell, 6.0 IU/cell, 7.0 IU/cell, 8.0 IU/cell, 9.0 IU/cell or 10.0 IU/cell.

54. The method of any of embodiments 1-53, wherein the input composition comprises at least at or about at least or about 50×10⁶ cells, 100×10⁶ cells, or 200×10⁶ cells.

55. The method of any of embodiments 1-54, wherein the recombinant nucleic acid encodes an antigen receptor.

56. The method of embodiment 55, wherein the antigen receptor is a transgenic T cell receptor (TCR).

57. The method of embodiment 55 or embodiment 56, wherein the antigen receptor is a chimeric antigen receptor (CAR).

58. The method of embodiment 57, wherein the chimeric antigen receptor (CAR) comprises an extracellular antigen-recognition domain that specifically binds to a target antigen and an intracellular signaling domain comprising an ITAM.

59. The method of embodiment 58, wherein the intracellular signaling domain comprises an intracellular domain of a CD3-zeta (CD3) chain.

60. The method of embodiment 58 or embodiment 59, further comprising a transmembrane domain linking the extracellular domain and the intracellular signaling domain.

61. The method of embodiment 60, wherein the transmembrane domain comprises a transmembrane portion of CD28.

62. The method of any of embodiments 58-61, wherein the intracellular signaling domain further comprises an intracellular signaling domain of a T cell costimulatory molecule.

63. The method of embodiment 62, wherein the T cell costimulatory molecule is selected from the group consisting of CD28 and 41BB.

64. The method of any of embodiments 55-63, wherein the antigen receptor specifically binds to an antigen associated with a disease or condition or specifically binds to a universal tag.

65. The method of embodiment 64, wherein the disease or condition is a cancer, and autoimmune disease or disorder, or an infectious disease.

66. The method of any of embodiments 1-65, wherein the method produces an output composition comprising T cells transduced with the recombinant nucleic acid.

67. The method of embodiment 66, wherein at least 30%, or at least 40%, at least 50%, at least 60%, at least 70% or at least 80% of the T cells in the output composition are transduced with the recombinant nucleic acid.

68. The method of embodiment 66 or embodiment 67, further comprising recovering or isolating from the output composition the transduced T cells produced by the method.

69. The method of embodiment 66 or embodiment 68, further comprising activating or expanding the cells of the output composition or the cells transduced by the method.

70. The method of embodiment 69, wherein activation and/or expansion is performed ex vivo.

71. The method of embodiment 69 or embodiment 70, wherein, subsequent to the incubation, the cells in the output composition are further incubated in the presence of one or more stimulating agent capable of activating T cells, inducing a signal through a TCR complex and/or inducing proliferation of T cells.

72. The method of embodiment 71, wherein the one or more stimulating agent is selected from the group consisting of CD3-binding molecules; CD28-binding molecules; recombinant IL-2; recombinant IL-15; and recombinant IL-7.

73. The method of embodiment 71 or embodiment 72, wherein the one or more stimulating agents comprise an anti-CD3 antibody and/or an anti-CD28 antibody.

74. The method of embodiment 69, wherein activation and/or expansion is performed in vivo.

75. The method of embodiment 69 or embodiment 74, wherein activation and/or expansion occurs in the presence of antigen specifically bound by the antigen receptor and/or is transgene-specific.

76. The method of embodiment 72 or embodiment 75, wherein, subsequent to the incubation, the cells in the output composition are not further incubated ex vivo in the presence of one or more stimulating agent, optionally consisting of CD3-binding molecules; CD28-binding molecules; recombinant IL-2; recombinant IL-15; and recombinant IL-7, and/or the cells in the output composition are not further incubated at a temperature greater than 30° C. for more than 24 hours.

77. A genetically engineered T cell produced by the method of any of embodiments 1-76.

78. A composition, comprising the genetically engineered T cell of embodiment 77 and a pharmaceutically acceptable carrier.

79. A method of treatment, the method comprising administering to a subject having a disease or condition the composition of embodiment 78.

80. The method of embodiment 79, wherein the composition is administered to the subject no more than 5 days after obtaining the sample from the subject.

81 The method of embodiment 80, wherein the composition is administered to the subject no more than 1 day, 2 days, 3 days or 4 days after obtaining the sample from the subject.

82. A method for adoptive cell therapy, comprising:

(a) enriching or isolating T cells from a sample obtained from subject having a disease or condition;

(b) transducing an input composition comprising the T cells with a viral vector particle by the method of any of embodiments 1-71, thereby producing an output composition comprising transduced cells, wherein the viral vector particle comprises a recombinant nucleic acid encoding an antigen receptor that specifically binds to an antigen associated with the disease or disorder;

(c) administering the output composition comprising the transduced cells to the subject for treating the disease or condition, wherein the output composition is administered to the subject no more than 9 days after obtaining the sample from the subject.

83. A method of adoptive cell therapy, comprising administering an output composition comprising T cells transduced with a recombinant nucleic acid to a subject for treating a disease or condition, wherein the output composition is produced by a method of any of embodiments 1-71.

84. The method of embodiment 82 or embodiment 83, wherein the output composition is administered to the subject no more than 1 day, 2 days, 3 days, 4 days or 5 days after obtaining the sample from the subject.

85. The method of any of embodiments 82-84, wherein prior to administering the composition, the transduced cells or the cells of the output composition are formulated in a pharmaceutically acceptable buffer.

86. The method of any of embodiments 82-85, wherein, prior to administering the output composition comprising transduced cells, the cells are cultured ex vivo for up to 24 hours, 2 days, 3 days, 4 days, 5 days, 6 days, 7 days, 8 days or 9 days after the transducing, said culturing occurring at a temperature greater than 30° C.

87. The method of any of embodiments 82-86, wherein, subsequent to the transduction, the output composition or cells comprising the transduced cells are cultured in the presence of one or more stimulating agent capable of activating T cells, inducing a signal through a TCR complex and/or inducing proliferation of T cells, thereby producing the composition comprising the transduced cells.

88. The method of any of embodiments 82-85, wherein, prior to administering the transduced cells, the output composition or cells comprising the transduced cells are not further incubated ex vivo in the presence of one or more stimulating agent and/or are not further incubated at a temperature greater than 30° C. for more than 24 hours.

89. The method of embodiment 87 or embodiment 88, wherein the one or more stimulating agent is selected from the group consisting of CD3-binding molecules; CD28-binding molecules; recombinant IL-2; recombinant IL-15; and recombinant IL-7, a vaccine comprising an antigen specifically recognized by the antigen receptor, and an anti-idiotype antibody that specifically binds the antigen receptor.

90. The method of embodiment 89, wherein the one or more stimulating agents comprise an anti-CD3 antibody and/or an anti-CD28 antibody.

91. The method of any of embodiments 82-90, wherein the cells of the output composition or the transduced cells are administered at a sub-optimal dose.

92. The method of any of embodiments 82-91, further comprising administering to the subject one or more agents to induce or enhance stimulation and/or expansion of the transduced T cells in vivo.

93. The method of embodiment 92, wherein the one or more agents is transgene-specific and/or stimulates or activates the cells via the expressed transgene, which optionally is or comprises an antigen receptor.

94. The method of embodiment 92 or embodiment 93, wherein the one or more agent is selected from among a vaccine comprising an antigen specifically recognized by the antigen receptor, an anti-idiotype antibody that specifically binds the antigen receptor or an agent capable of chemically inducing dimerization of the antigen receptor.

95. The method of embodiment 92, wherein the one or more agents is an immunomodulatory agent; an immune checkpoint inhibitor; an inhibitor of extracellular adenosine or adenosine receptor, optionally an A2aR receptor; a kynurenine pathway modulator, and modulators of signaling pathways, e.g., kinase inhibitors.

96. A composition, comprising a population of primary human T cells genetically engineered to express a chimeric antigen receptor (CAR) or transgenic TCR that specifically binds to a target antigen, wherein:

the population comprises a plurality of resting T cells; and

the plurality of resting T cells comprise at least 7.5% percent of the genetically engineered cells in the composition.

97. The composition of embodiment 96, wherein the genetically engineered resting T cells comprise at least 10%, at least 20%, at least 30%, at least 40%, at least 50%, at least 60%, at least 70%, at least 80%, or at least 90% of the genetically engineered cells in the composition.

98. The composition of embodiment 96 or embodiment 97, wherein the resting T cells are surface negative for a T cell activation marker selected from the group consisting of HLA-DR, CD25, CD69, CD71, CD40L (CD154) and 4-1BB (CD137); lack intracellular expression of a cytokine selected from the group consisting of IL-2, IFN-gamma and TNF-alpha; are in the G0 or G₀G₁ₐ stage of the cell cycle; and/or contain an active SAMHD1.

99. The composition of embodiment 98, wherein the resting T cells are surface negative for CD25 and CD69 (CD25-/CD69-).

100. The composition of any of embodiments 96-99, wherein the resting T cells comprise CD4+ and/or CD8+ T cells.

101. The composition of any of embodiments 96-100, wherein the target antigen is associated with a disease or disorder.

102. The composition of embodiment 100, wherein the disease or disorder is an infectious disease or condition, an autoimmune disease, an inflammatory disease or a cancer.

103. The composition of any of embodiments 96-102, wherein the target antigen is selected from the group consisting of B cell maturation antigen (BCMA), carbonic anhydrase 9 (CAIX), tEGFR, Her2/neu (receptor tyrosine kinase erbB2), CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L-cell adhesion molecule (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, G Protein Coupled Receptor 5D (GPCR5D), ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag.

104. The composition of any of embodiments 96-103, wherein the primary human T cells are genetically engineered to express a CAR, which comprises an extracellular antigen-recognition domain that specifically binds to a target antigen and an intracellular signaling domain comprising an ITAM.

105. The composition of embodiment 104, wherein the intracellular signaling domain comprises an intracellular domain of a CD3-zeta (CD3) chain.

106. The composition of embodiment 104 or embodiment 105, wherein the CAR further comprises a transmembrane domain linking the extracellular domain and the intracellular signaling domain.

107. The composition of embodiment 106, wherein the transmembrane domain comprises a transmembrane portion of CD28.

108. The composition of any of embodiments 104-107, wherein the intracellular signaling domain of the CAR further comprises an intracellular signaling domain of a T cell costimulatory molecule.

109. The composition of embodiment 108, wherein the T cell costimulatory molecule is selected from the group consisting of CD28 and 41BB.

110. The composition of any of embodiments 96-108, comprising a pharmaceutically acceptable carrier.

### VI. EXAMPLES

The following examples are included for illustrative purposes only and are not intended to limit the scope of the invention.

### Example 1: Assessment of Lentiviral Transduction of Primary Human T Cells without Prior Cell Activation

Lentiviral transduction of primary CD4+ and CD8+ T cells, enriched from a human leukapheresis sample, was carried out without first subjecting the cells to an ex vivo T cell activation step prior to transduction.

A human leukapheresis sample enriched in mononuclear cells was obtained from a whole blood sample from a subject using a leukapheresis collection system. Blood was processed to yield a leukapheresis sample containing mononuclear cells, autologous plasma and an anti-coagulant, ACD-A (anti-coagulant citrate dextrose A).

Cells of the leukapheresis sample were washed and resuspended in a buffer for use in affinity-based selection, the buffer containing Phosphate Buffered Saline (PBS), EDTA and human serum albumin For immunoaffinity-based selection of T cells, the washed cells in the selection buffer were incubated for 30 minutes at room temperature with magnetic beads coupled to monoclonal antibodies for selection and subjected to selection using a magnetic separation column. The enriched T cells were resuspended in cryopreservation media and cells were cyropreserved and stored in liquid nitrogen until further use. Cryopreserved CD4+ and CD8+ T cells were thawed, washed and resuspended in media for transduction.

The T cells were then contacted with lentiviral vector particles without incubating the cells with a T cell activation agent, e.g. without incubating the cells with anti-CD3 and anti-CD28 reagents. For transduction, the T cells were added to individual wells of a 24-well plate in a 200 μL volume. Lentiviral vector particles containing a nucleic acid encoding a transgene (in this case encoding an exemplary chimeric antigen receptor (CAR) and a truncated EGFR, EGFRt, sequence for use as a transduction marker, separated from the CAR sequence by a self-cleaving T2A sequence) that had been premixed with a polycation transduction adjuvant were added to wells at 2-fold serial dilutions. The final volume per well was adjusted to 1.1 mL. The compositions were subjected to centrifugation for approximately 1 hour at and then incubated for 24 hours at 37° C.

As a control, transduction also was performed as described above using CD4+ and CD8+ T cells thawed from a cryopreserved composition, except that, prior to transduction, the T cells were activated by culturing at 37° C. for approximately 24 hours with one of two different activation reagents each containing magnetic beads coated with anti-CD3 and anti-CD28 antibody fragments (designated reagent 1 or reagent 2).

Following transduction in each of the above conditions, cells of each compositions were cultured at 37° C. in the presence of media and IL-2 for 48 hours.

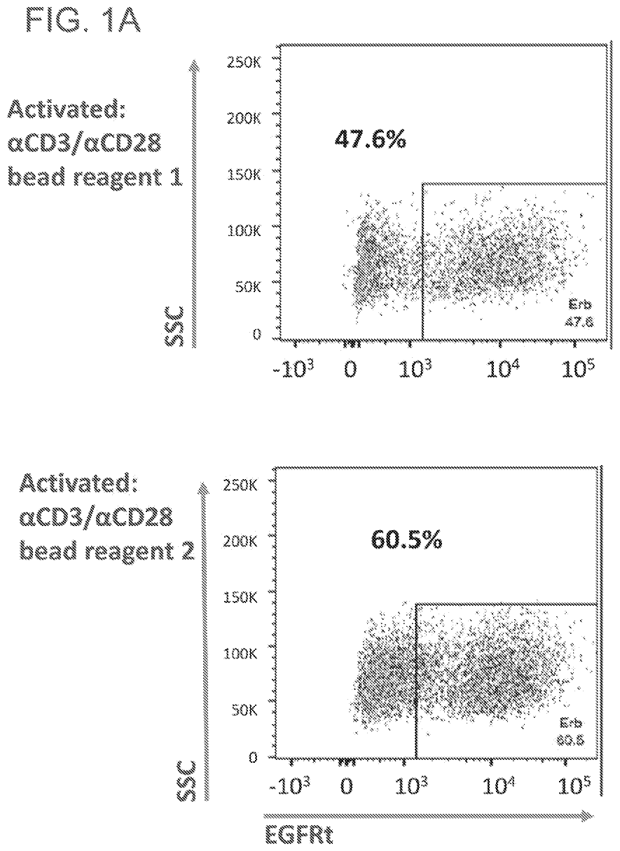

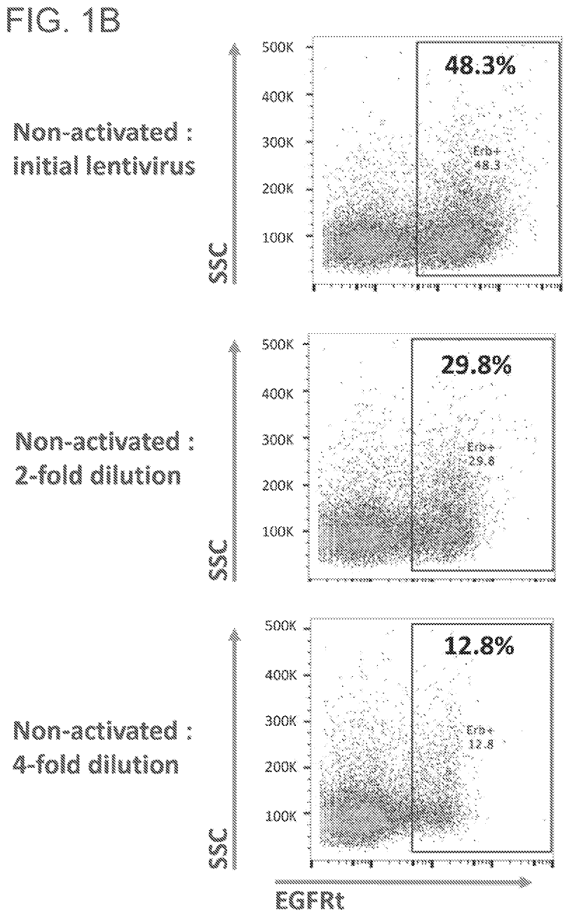

As a measure of transduction efficiency, a flow cytometry-based assay was used to determine the percentage of cells positive for surface expression of the CAR (in this case detected using an anti-EGFR antibody that recognizes the surrogate marker EGFRt). As shown in FIGS. 1A-1B, in this study transduction efficiency (as reflected by EGFRt surface expression) without activation was comparable to the transduction efficiency in cells that were activated with anti-CD3/anti-CD28 reagents prior to transduction. The results showed that comparable transduction of T cells could be achieved using lentiviral vectors without prior incubation with an agent for ex vivo activation of T cells.

### Example 2: Large Scale Process for Lentiviral Transduction of Primary Human T Cells without Prior Cell Activation

This Example describes transduction of CD4+ and CD8+ selected T cells from a healthy donor apheresis via a large-scale process, in which cells were obtained, affinity-selected, transduced (with or without prior activation) and cultured or incubated under various conditions. The wash, affinity-based selection and transduction were performed in a substantially vertical centrifugal processing chamber, for example, as described in International Publication Number WO2016/073602.

1. Sample Collection and Leukapheresis

Autologous peripheral blood mononuclear cells (PBMCs) were collected from a patient using a leukapheresis collection system. Blood was processed to yield a sample containing mononuclear cells and approximately 1/3 volume autologous plasma and an anti-coagulant, ACD-A (anti-coagulant citrate dextrose A). The leukapheresis sample was stored sealed at 2-8° C., for approximately 24 hours.

2. Leukapheresis Wash

The leukapheresis sample was sterilely transferred to a transfer pack. Cells of the leukapheresis sample were washed and resuspended in a selection buffer for affinity-based selection, the buffer containing PBS, EDTA, and human serum albumin. The wash was carried out within a sterile, single-use disposable kit sold by Biosafe SA for use in regenerative medicine, which included a centrifugal processing chamber (A-200F). The transfer pack containing the cells and a bag containing the buffer were sterilely connected to the kit, which was placed in association with a Sepax® 2 processing unit. Two (2) wash cycles were performed, each carried out at an RCF at the internal wall of the cavity of approximately 200 g for 180 seconds, followed by final resuspension of the cells in 20 mL The cells were retained in the processing cavity of the centrifuge chamber at the end of the protocol, for subsequent incubation with reagents for affinity-based selection.

3. Affinity-Based Selection

CD4 and CD8 T cells were enriched by immunoaffinity-based selection using magnetic beads coupled to monoclonal antibodies, which were added to the washed leukapheresis within the centrifugal chamber. Beads were mixed in selection buffer described above, which then was sterilely connected to the apparatus. A program was run on the Sepax® 2 unit which caused the bead mixture and selection buffer to be drawn into the chamber with the washed cells, and the contents of the chamber to be mixed for 30 minutes.

At the end of the program, the Sepax® 2 unit caused pelleting of the cells and expulsion of excess buffer/beads, washing of the pelleted cells, and resuspension in selection buffer. The program caused the washed cells to be collected into a transfer pack.

The cells then were passed from the transfer pack, through a closed, sterile system of tubing lines and a separation column, in the presence of a magnetic field using standard methods, to separate cells that had bound to the CD4- and/or CD8-specific reagents. These magnetically-labeled cells then were collected in a transfer pack for further processing.

4. Transduction

Transduction with a lentiviral vector was carried out in the centrifugal chamber integral to the kit, placed in association with the Sepax® processing unit. The selected cells were either transduced (transduced at t=0) or were activated using an anti-CD3/CD28 reagent(s) (activated t=0 and transduced at t=24 h).

For cells that were first activated, selected cells were washed and resuspended in complete media using the Sepax® 2 system, which then was combined with the anti-CD3/28 reagent(s) in the cavity of the chamber at room temperature. Following the incubation, the incubated material was transferred via the Sepax® 2 unit into a cell culture bag, which then was incubated at 37° C. for 24 hours.

To initiate transduction, the following steps were carried out.

A transduction reagent solution was prepared containing complete media (X-VIVO-15 containing 5% (v/v) human serum, 1.6 mg/mL N-Acetylcysteine (NAC) and 100 IU/mL IL-2) and a polycation in an amount sufficient for a final concentration during transduction initiation of 10 μg/mL), viral vector particles (approximately 3.2 IU/cell), and, where applicable, air. The transduction reagent solution was aseptically transferred to a centrifuge bag.

A product bag containing a composition with approximately 200×10⁶ selected cells or 200×10⁶ activated cells was sterilely-connected to a single-use disposable kit that was placed in association with a Sepax® 2 processing unit. An automated cycle was run on the Sepax® 2, facilitating drawing in of the composition containing the cells into the cavity of the chamber, spinning of the composition on the Sepax® 2 to pellet the cells, and removal of the appropriate volume of liquid required to achieve a desired volume, e.g., 10 mL. The content of the transduction reagent solution then was drawn into the cavity of the chamber with the cells. The resulting 200 mL volume (containing the cells and virus) then was transferred into a centrifuge bag.

To initiate transduction, the centrifuge bag containing the virus and cells was removed and sterilely-connected at an input position of the kit, and transferred to the cavity of the chamber. The virus and cells were spun in the cavity of the chamber at an approximate RCF at the internal wall of the cavity of 1600 g. The spin was carried out at the indicated speed for 1 hour. The transduced material was then transferred into an output bag. Complete media was transferred to the chamber, mixed for 60 seconds, and then transferred from the processing cavity to the output bag to yield a total volume of 200 mL in the output bag containing the transduced material.

The cells transduced post-selection, without a prior activation step, were then (1) immediately activated with anti-CD3/anti-CD28 reagent in culture media supplemented with FBS, 100 IU/mL IL-2 and 10 IU/mL IL-15) and harvested on day 9; (2) incubated at 37° C., 5% CO₂ for 24 hours, then activated with anti-CD3/anti-CD28 in culture media and harvested on day 9; (3) maintained without activation by culture at 37° C., 5% CO₂ for 3 days; (4) incubated 37° C., 5% CO₂ for 24 hours, cryopreserved, thawed and activated with anti-CD3/anti-CD28 reagent in culture media and harvested on day 14. For control cells that were activated prior to transduction and then transduced 24 hours later, the cells were expanded in the presence of culture media (without activation) and harvested at day 9. For each condition, cells were cultured at 37° C., 5% CO₂.

Table 1 summarizes the tested groups and the incubation conditions following transduction.

TABLE 1
Transduction Groups

| Conditions | Description |
|---|---|
| Activated t = 0 and transduced | Activated post-selection, transduced |
| at t = 24 h (Act0_Tx24; control) | 24 h later, harvested on day 9 |
| Transduced at t = 0 and | Transduced post-selection, then |
| activated t = 0 (Tx_Act_0) | incubated with activation reagent |
| | immediately after transduction, and |
| | harvested on day 9 |
| Transduced at t = 0 and | Transduced post-selection, incubated |
| activated t = 24 h (Tx0_Act24) | with activation reagent 24 h later, and |
| | harvested on day 9 |
| Transduced at t = 0, | Transduced post-selection, |
| Cryopreserved, Activated | cryopreserved 24 h later, thawed and |
| t = 48 h (Tx0_Cryopreserved) | incubated with activation reagent at on |
| | day 2, and harvested on day 14 |
| Transduced at t = 0, not | Transduced post-selection (no |
| activated (Tx_No_Act) | activation) and cultured for 3 days. |

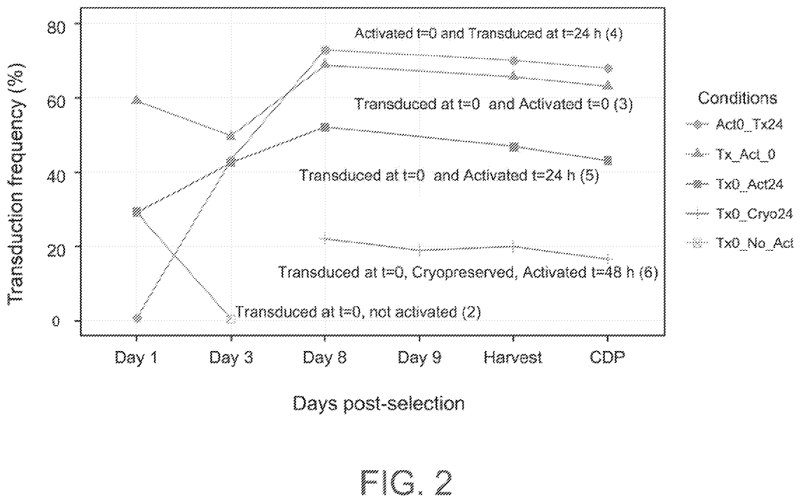

Transduction frequency at various days post-selection of cells from leukapheresis was measured as described in Example 1. As shown in FIG. 2, comparable transduction frequencies (as measured by surface marker expression) were observed when T cells were first transduced and then immediately activated compared with T cells that were first activated for 24 h before being transduced (63% vs. 68% respectively in cryopreserved product harvested after 9 days post-selection). This result demonstrated that relatively high transduction efficiency could be obtained without activation of T cells prior to transduction.

Whereas prior T cell activation was not required for transduction (gene integration), results suggested that in this study, incubation of the cells with an activating agent observed to be important for sustained surface expression of the recombinant protein. In cells that were not activated, surface EGFRt expression (surface surrogate marker for CAR expression) was transient; detected frequency of surface expression of EGFRt (indicative of transduction) peaked 24h post-transduction at 29% and dropped to <1% by day 3 post-transduction.

In addition, FIG. 2 showed that even when incubation with the activation reagent was delayed 24 hours (as compared to cells activated prior to transduction) resulted in a sufficiently higher frequency of the marker (indicative of transduction frequency) of 43%. Immediate incubation with activation reagent (magnetic beads with anti-CD3/CD28) following transduction resulted in higher frequencies of cells displaying surface expression of the transduction marker than when activation was delayed by 24 h (63% vs. 43% respective frequency measured in the cryopreserved product harvested 9 days post-selection). Cryopreservation of transduced cells (24 h post-transduction) prior to subsequent thaw and incubation with the activation reagent resulted in a decrease in transduction marker frequency as compared to cells transduced and incubated with the activation reagent without cryopreservation (16% vs. 43% respective transduction frequency measured in the cryopreserved product).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ ID NO: | Sequence | Description |
|---|---|---|
| 1 | MSDPRERIPPGNSGEETIGEAFEWLNRTVEEINREAVNHLPRELIFQV | SIVmac |
| | WQRSWEYWHDEQGMSQSYVKYRYLCLMQKALFMHCKKGCRCLGEGHGA | Vpx |
| | GGWRPGPPPPPPPGLA | |
| 2 | MTDPRERIPPGNSGEETIGEAFEWLHNTVEALNQTAVQHLPRELIFQV | SIVsm |
| | WRRCWEYWVDEQGYSPSYAKYRYVQLMQKAMFQHFRKGCTCRGEGHSQ | Vpx |
| | GGWRTGPPPPPPPGLA | |
| 3 | MSDPRERIPPGNSGEETIGEAFDWLDRTVEEINRAAVNHLPRELIFQV | SIVrcm |
| | WRRSWEYWHDEMGMSVSYTKYRYLCLIQKAMFMHCKKGCRCLGGEHGA | Vpx |
| | GGWRPGPPPPPPPGLA | |
| 4 | MADPRERVPPGNSGEETIGEAFEWLDRTIEALNREAVNHLPRELIFQV | HIV-2 |
| | WQRSWAYWHDEQGMSTSYTKYRYLCIMQKAVYIHFKKGCTCLGRGHGP | Vpx |
| | GGWRPGPPPPPPPGLV | |
| 5 | MERYPPSHPPHFTSRTVPMTRLALQQAMQDLNEEALKHFTREELWGVW | SIVdeb |
| | NHCVDLPAQPDWTGEQAWAASVIDYIKIVQRMLWLHLREACFHREREA | Vpr |
| | TRRYPNIRPLTGRNREVRDGE | |
| 6 | MERVPPSHRPPWHSRVVPTTMQQAQQAMWDLNEEAEKHFSREELRGIW | SIVmus |
| | NDVTELPADPNWTVDQAAIACAIDYIRRTQTLLFRHYREGCYHRYSNT | Vpr |
| | IRRYPNIRPLRGTQAPPSNSMPNADPTPPLRPSRYRMDE | |
| 7 | MASGRDPREERPGELEIWDLSREPWDEWLRDMLTELNQEAQRHFGREL | SIVagm |
| | LFQVWNYCQEEGERQNVPMQERAYKYYKLVQRALFVHFRCGCRRRQPF | Vpr |
| | EPYEERRNGQGGGRAGRVPPGLD | |
| 8 | MASGRDPREERPGEVEIWDLSREPWDEWLRDMLAELNREAQRHFGREL | SIVagm |
| | LFQVWNYCQEEGERQNIPMQERAYKYYRLVQKALFVHIRCGCRRRQPF | Vpr |
| | EPYEERRNGQGGGRAERVPPGLD | |
| 9 | MERYPPSHPPHFTSRTVPMTRLALQQAMQDLNEEALKHFTREELWGVW | SIVdeb |
| | NHCVDLPAQPDWTGEQAWAASVIDYIKIVQRMLWLHLREACFHREREA | Vpr |
| | TRRYPNIRPLTGRNREVRDGE | |
| 10 | MERLPPSHPPALVSRMAETTQRQLQEAVWDITEEARKHFSKEEITGIW | SIVden |
| | DHCWSLPALPHWTEGQVMAAAVIDFIRIMQKEIWKHYRVGCFHREAER | Vpr |
| | VRHYPNIRPLRPRREEP | |
| 11 | MERLPPSHPLPLTSRLIPTPRPQLERLMREVRDEMLIHFIRDEVIGVW | SIVtal |
| | NHCVELPADPDWTGEQAWAASVIDFARKGNQMLLLHFQQGCYHEQQRR | Vpr |
| | TPHYPNIRPLSGRGERTMQ | |
| 12 | MERIPPSHPMPWLSRRVPTTMAIAQNALWEINEEAEKHFSRDELRGIW | SIVgsn |
| | HDVTELPADPDWTVDQAAIACAIDYIRVQTLLFRHFKDGCFHRVNLSG | Vpr |
| | RYPAIRPSRGTAPPDSNSVSHADPEQPRRPSRYRMDE | |
| 13 | MERTPPSHPLPWISRRVPITMAVAQSAMWEINEEAEKHFSREELRGIW | SIVgsn |
| | HDVTELPADPDWTVDQAAIACAIDYVRRVQTLLFRHFRDGCFHRYNRI | Vpr |
| | VRRYPVIRPLRGTAPPDSNSVPHADPEQPRRPSRYRMDE | |
| 14 | MEQPPQSHPLHWTSRMVPIERQALQAAIWELNEEALKHFSREELRGIW | SIVmon |
| | EQVTELPADPAWNADQAWAACAIDYTRWVQTILYRHYREGCYHRYAEQ | Vpr |
| | IRRYPVLRPMRGTAPGPTSSVPQADPDNPRRPSRYRMDE | |
| 15 | MAEAFFNPSQHVQGTPWFFIPRNVELTPNVINVTVKAELVVTEASKHF | SIVsyk |
| | TPQEIYGVWNQSLNEEAGTDSPTMAWERTMLDMVRALNLMLFEHFAAG | Vpr |
| | CPQRTRYARHRGYPHPS | |
| 16 | MAERAPEAPEGAGEVGLEQWLETSLERINREARLHFHPEFLFRLWNTC | SIVmnd2 |
| | VEHWHDRHQRSLDYAKYRYLLLMHKAMYTHMQQGCPCRNGRPRGPPPP | Vpx |
| | GMA | |
| 17 | MAERQSVERAPAEPMGAGEVELEEWLQRSLLRINQEARLHFHPEFLFR | SIVdr1 |
| | LWNTCMEHYHDALQLSFTYSKYRYLLLLQKAMFMHFQQGCSCLQGRHP | Vpx |
| | PPLRPAGDRLPPPPPPP | |
| 18 | MSDPRERIPPGNSGEETIGEAFEWLNRTVEEINREAVNHLPRELIFQV | SIVmne |
| | WQRSWEYWHDEQGMSPSYVKYRYLCLIQKALFMHCKKGCRCLGEGHGA | Vpx |
| | GGWRPGPPPPPPPGLA | |
| 19 | MQRADSEQPSKRPRCDDSPRTPSNTPSAEADWSPGLELHPDYKTWGPE | Human |
| | QVCSFLRRGGFEEPVLLKNIRENEITGALLPCLDESRFENLGVSSLGE | SAMHD1 |
| | RKKLLSYIQRLVQIHVDTMKVINDPIHGHIELHPLLVRIIDTPQFQRL | |
| | RYIKQLGGGYYVFPGASHNRFEHSLGVGYLAGCLVHALGEKQPELQIS | |
| | ERDVLCVQIAGLCHDLGHGPFSHMFDGRFIPLARPEVKWTHEQGSVMM | |
| | FEHLINSNGIKPVMEQYGLIPEEDICFIKEQIVGPLESPVEDSLWPYK | |
| | GRPENKSFLYEIVSNKRNGIDVDKWDYFARDCHHLGIQNNFDYKRFIK | |
| | FARVCEVDNELRICARDKEVGNLYDMFHTRNSLHRRAYQHKVGNIIDT | |
| | MITDAFLKADDYIEITGAGGKKYRISTAIDDMEAYTKLIDNIFLEILY | |
| | STDPKLKDAREILKQIEYRNLFKYVGETQPIGQIKIKREDYESLPKEV | |
| | ASAKPKVLLDVKLKAEDFIVDVINMDYGMQEKNPIDHVSFYCKTAPNR | |
| | AIRITKNQVSQLLPEKFAEQLIRVYCKKVDRKSLYAARQYFVQWCADR | |
| | NFTKPQDGDVIAPLITPQKKEWNDSTSVQNPTRLREASKSRVQLFKDD | |
| | PM | |
| 20 | LQSRPEPTAPPEESFRFGVETTIPPQKQEPIDKELYPLISLRSLFGND | HIV-1 p6 |
| | PSSQ | |
| 21 | PMAQVHQGLMPTAPPEDPAVDLLKNYMQLGKQQREKQRESREKPYKEV | P6 Gag |
| | TEDLLHLNSLFGGDQ | |
| 22 | DXAXXLL | P6 Gag |
| | | packaging |
| | | motif |
| 23 | DPAVDLL | P6 |
| | | packaging |
| | | motif |
| 24 | DPAVDLLKNY | P6 |
| | | packaging |
| | | motif |
| 25 | LQSRPEPTAPPEESDPAVDLLTIPPQKQEPIDKELYPLISLRSLFGND | Hybrid or |
| | PSSQ | chimeric |
| | | p6 domain |
| 26 | WXXXXXXXXXXXAXXH | Wx40x20x3 |
| | | AOxH |
| | | motif |
| 27 | ESKYGPPCPPCP | spacer |
| | | (IgG4hing |
| | | e) (aa) |
| 28 | GAATCTAAGTACGGACCGCCCTGCCCCCCTTGCCCT | spacer |
| | | (IgG4hing |
| | | e) (nt) |
| 29 | ESKYGPPCPPCPGQPREPQVYTLPPSQEEMTKNQVSLTCLVKGFYPSD | Hinge-CH3 |
| | IAVEWESNGQPENNYKTIPPVLDSDGSFFLYSRLIVDKSRWQEGNVFS | spacer |
| | CSVMHEALHNHYTQKSLSLSLGK | |
| 30 | ESKYGPPCPPCPAPEFLGGPSVFLFPPKPKDTLMISRTPEVTCVVVDV | Hinge- |
| | SQEDPEVQFNWYVDGVEVHNAKTKPREEQFNSTYRVVSVLTVLHQDWL | CH2-CH3 |
| | NGKEYKCKVSNKGLPSSIEKTISKAKGQPREPQVYTLPPSQEEMTKNQ | spacer |
| | VSLTCLVKGFYPSDIAVEWESNGQPENNYKTTPPVLDSDGSFFLYSRL | |
| | TVDKSRWQEGNVFSCSVMHEALHNHYTQKSLSLSLGK | |
| 31 | RWPESPKAQASSVPTAQPQAEGSLAKATTAPATTRNTGRGGEEKKKEK | IgD- |
| | EKEEQEERETKTPECPSHTQPLGVYLLTPAVQDLWLRDKATFTCFVVG | hinge-Fc |
| | SDLKDAHLTWEVAGKVPTGGVEEGLLERHSNGSQSQHSRLTLPRSLWN | |
| | AGTSVTCTLNHPSLPPQRLMALREPAAQAPVKLSLNLLASSDPPEAAS | |
| | WLLCEVSGFSPPNILLMWLEDQREVNTSGFAPARPPPQPGSTTFWAWS | |
| | VLRVPAPPSPQPATYTCVVSHEDSRTLLNASRSLEVSYVTDH | |
| 32 | LEGGGEGRGSLLTCGDVEENPGPR | T2A |
| 33 | MLLLVTSLLLCELPHPAFLLIPRKVCNGIGIGEFKDSLSINATNIKHF | tEGFR |
| | KNCTSISGDLHILPVAFRGDSFTHTPPLDPQELDILKTVKEITGFLLI | |
| | QAWPENRTDLHAFENLEIIRGRTKQHGQFSLAVVSLNITSLGLRSLKE | |
| | ISDGDVIISGNKNLCYANTINWKKLFGTSGQKTKIISNRGENSCKATG | |
| | QVCHALCSPEGCWGPEPRDCVSCRNVSRGRECVDKCNLLEGEPREFVE | |
| | NSECIQCHPECLPQAMNITCTGRGPDNCIQCAHYIDGPHCVKTCPAGV | |
| | MGENNTLVWKYADAGHVCHLCHPNCTYGCTGPGLEGCPTNGPKIPSIA | |
| | TGMVGALLLLLVVALGIGLFM | |
| 34 | FWVLVVVGGVLACYSLLVTVAFIIFWV | CD28 |
| | | (amino |
| | | acids |
| | | 153-179 |
| | | of |
| | | Accession |
| | | No. |
| | | P10747) |
| 35 | IEVMYPPPYLDNEKSNGTIIHVKGKHLCPSPLFPGPSKP | CD28 |
| | FWVLVVVGGVLACYSLLVTVAFIIFWV | (amino |
| | | acids |
| | | 114-179 |
| | | of |
| | | Accession |
| | | No. |
| | | P10747) |
| 36 | RSKRSRLLHSDYMNMTPRRPGPTRKHYQPYAPPRDFAAYRS | CD28 |
| | | (amino |
| | | acids |
| | | 180-220 |
| | | of |
| | | P10747) |
| 37 | RSKRSRGGHSDYMNMTPRRPGPTRKHYQPYAPPRDFAAYRS | CD28 (LL |
| | | to GG) |
| 38 | KRGRKKLLYIFKQPFMRPVQTTQEEDGCSCRFPEEEEGGCEL | 4-1BB |
| | | (amino |
| | | acids |
| | | 214-255 |
| | | of |
| | | Q07011.1) |
| 39 | RVKFSRSADAPAYQQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 40 | RVKFSRSAEPPAYQQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 41 | RVKFSRSADAPAYKQGQNQLYNELNLGRREEYDVLDKRRGRDPEMGGK | CD3 zeta |
| | PRRKNPQEGLYNELQKDKMAEAYSEIGMKGERRRGKGHDGLYQGLSTA | |
| | TKDTYDALHMQALPPR | |
| 42 | RKVCNGIGIGEFKDSLSINATNIKHFKNCTSISGDLHILPVAFRGDSF | tEGFR |
| | THTPPLDPQELDILKTVKEITGFLLIQAWPENRTDLHAFENLEIIRGR | |
| | TKQHGQFSLAVVSLNITSLGLRSLKEISDGDVIISGNKNLCYANTINW | |
| | KKLFGTSGQKTKIISNRGENSCKATGQVCHALCSPEGCWGPEPRDCVS | |
| | CRNVSRGRECVDKCNLLEGEPREFVENSECIQCHPECLPQAMNITCTG | |
| | RGPDNCIQCAHYIDGPHCVKTCPAGVMGENNTLVWKYADAGHVCHLCH | |
| | PNCTYGCTGPGLEGCPTNGPKIPSIATGMVGALLLLLVVALGIGLFM | |
| 43 | EGRGSLLTCGDVEENPGP | T2A |
| 44 | GSGATNFSLLKQAGDVEENPGP | P2A |
| 45 | ATNFSLLKQAGDVEENPGP | P2A |
| 46 | QCTNYALLKLAGDVESNPGP | E2A |
| 47 | VKQTLNFDLLKLAGDVESNPGP | F2A |
| 48 | -PGGG-(SGGGG)5-P- wherein P is proline, G is | Linker |
| | glycine and S is serine | |
| 49 | GSADDAKKDAAKKDGKS | Linker |
| 50 | atgcttctcctggtgacaagccttctgctctgtgagttaccacaccca | GMCSFR |
| | gcattcctcctgatccca | alpha |
| | | chain |
| | | signal |
| | | sequence |
| 51 | MLLLVTSLLLCELPHPAFLLIP | GMCSFR |
| | | alpha |
| | | chain |
| | | signal |
| | | sequence |
| 52 | MALPVTALLLPLALLLHA | CD8 alpha |
| | | signal |
| | | peptide |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed:

1. A method for transducing T cells, the method comprising incubating a viral vector particle comprising a recombinant nucleic acid and an input composition comprising a plurality of T cells, said plurality of T cells having been obtained from a sample from a subject, wherein:
the incubating is initiated no more than 24 hours after obtaining the sample from the subject; and/or
prior to the incubating, the T cells have not been subjected to a temperature greater than or greater than about 15° C., about 18° C., about 22° C. or about 25° C. for a duration of more than 1 hour, 2 hours, 4 hours, 6 hours, 8 hours, 12 hours, or 24 hours after obtaining the sample from the subject; and/or
prior to the incubating, the T cells have not been subjected to a temperature of, of about, greater than, or greater than about 37°±2.0° C. for a duration of more than 15 minutes, 30 minutes, 1 hour or 2 hours after obtaining the sample from the subject.

2. The method of claim 1, wherein the incubating is initiated no more than or no more than about 1 hour, 3 hours, 6 hours, 12 hours or 18 hours after obtaining the sample from the subject.

3. The method of claim 1 or claim 2, wherein, prior to said incubation, the method does not comprise stimulating the T cells under conditions that promote cell activation.

4. The method of any of claims 1-3, wherein, prior to said incubating, the input composition has not been subjected to an ex vivo stimulation comprising incubation greater than or greater than about 37°±2.0° C. and/or incubation in the presence of an agent or agents capable of activating T cells, CD4+ T cells, and/or CD8+ T cells, incubation in the presence of an agent or agents capable of inducing a signal through a TCR complex and/or incubation in the presence of an agent or agents capable of inducing proliferation of T cells, CD4+ T cells, and/or CD8+ T cells; CD3-binding molecules; CD28-binding molecules; recombinant IL-2; recombinant IL-15; and recombinant IL-7.

5. The method of any of claims 1-4, wherein, prior to said incubating, no more than 5%, 10%, 20%, 30%, or 40% of the T cells are activated cells, express a surface marker selected from the group consisting of HLA-DR, CD25, CD69, CD71, CD40L and 4-1BB; comprise intracellular expression of a cytokine selected from the group consisting of IL-2, IFN-gamma, TNF-alpha, are in the G1 or later phase of the cell cycle and/or are capable of proliferating.

6. A method for transducing T cells, the method comprising incubating a viral vector particle comprising a recombinant nucleic acid and an input composition comprising T cells, said T cells having been obtained from a sample from a subject, wherein, prior to said incubating, the T cells or input composition have not been subjected to an ex vivo stimulation comprising incubation greater than or greater than about 37°±2.0° C. and/or incubation in the presence of an agent or agents-capable of activating T cells, CD4+ T cells, and/or CD8+ T cells, incubation in the presence of an agent or agents capable of inducing a signal through a TCR complex and/or incubation in the presence of an agent or agents capable of inducing proliferation of T cells, CD4+ T cells, and/or CD8+ T cells; CD3-binding molecules; CD28-binding molecules; recombinant IL-2; recombinant IL-15; and recombinant IL-7.

7. The method of claim 4 or claim 6, wherein the one or more agents comprise an anti-CD3 antibody and/or an anti-CD28 antibody.

8. A method for transducing T cells, the method comprising incubating a viral vector particle comprising a recombinant nucleic acid and an input composition comprising T cells, said T cells having been obtained from a sample from a subject, wherein, prior to the incubation, no more than 5%, 10%, 20%, 30%, or 40% of the T cells are activated cells, express a surface marker selected from the group consisting of HLA-DR, CD25, CD69, CD71, CD40L and 4-1BB; comprise intracellular expression of a cytokine selected from the group consisting of IL-2, IFN-gamma, TNF-alpha, and/or are in the G1 or later phase of the cell cycle.

9. The method of claim 5 or claim 8, wherein no more than 10% of T cells in the input composition comprise a T cell activation marker selected from the group consisting of HLA-DR, CD25, CD69, CD71, CD40L, and 4-1BB immediately prior to the incubation.

10. The method of any of claims 1-9, wherein, prior to said incubating, greater than 5%, 10%, 20%, 30%, or 40% of the T cells express the low-density lipid receptor (LDL-R).

11. The method of any of claims 1-9, wherein the subject is a human.

12. The method of any of claims 1-8, wherein the T cells have not been and/or are not maintained at a temperature of at 2° C. to 8° C. for more than 48 hours prior to the incubating.

13. The method of any of claims 1-12, wherein the sample is a blood sample.

14. The method of any of claims 1-12, wherein the sample is a leukapheresis sample.

15. The method of claim 1-14, wherein the T cells are unfractionated T cells, are enriched or isolated CD3+ T cells, are enriched or isolated CD4+ T cells or are enriched or isolated CD8+ T cells.

16. The method of any of claims 1-15, wherein the T cells have been selected or enriched from the sample from the subject.

17. The method of any of claims 1-16, further comprising, prior to the incubation, obtaining the sample from the subject and, optionally, selecting or enriching the T cells from the sample, which optionally generates an enriched composition and/or generates the input composition.

18. The method of any of claims 1-17, wherein the percentage of T cells in the input composition is greater than or greater than about 75%, 80%, 85%, 90%, 95% T cells.

19. The method of any of claims 3-18, wherein the T cells comprise CD4+ or CD8+ cells.

20. The method of any of claims 3-18, wherein the T cells comprise CD4+ and CD8+ cells.

21. The method of claim 20, wherein the ratio of the CD4+ cells to the CD8+ cells is or is about 1:1, 1:2, 2:1, 1:3 or 3:1.

22. The method of any of claims 1-21, wherein:
the sample comprises serum or plasma at a concentration of at least or at least about 10% (v/v), at least or at least about 15% (v/v), at least or at least about 20% (v/v), at least or at least about 25% (v/v), at least or at least about 30% (v/v), at least or at least about 33% (v/v), at least or at least about 35% (v/v), or at least or at least about 40% (v/v); and/or
prior to the incubating, the sample has been contacted ex vivo with serum or plasma at a concentration of at least or at least about 10% (v/v), at least or at least about 15% (v/v), at least or at least about 20% (v/v), at least or at least about 25% (v/v), at least or at least about 30% (v/v), at least or at least about 33% (v/v), at least or at least about 35% (v/v), or at least or at least about 40% (v/v).

23. The method of any of claims 1-22, wherein:
the sample comprises serum or plasma at a concentration of at least or at least about 30% (v/v); and/or
prior to the incubating, the sample has been contacted ex vivo with serum or plasma at a concentration of at least or at least about 30% (v/v).

24. The method of claim 22 or claim 23, wherein the serum or plasma is human.

25. The method of any of claims 22-24, wherein the serum or plasma is autologous to the subject.

26. The method of any of claims 1-22, wherein the sample comprises an anticoagulant.

27. The method of claim 26, wherein the anticoagulant comprises free citrate ion.

28. The method of any of claims 1-27, wherein, prior to the incubating, the method comprises cryopreserving the T cells, optionally T cells of the sample or of the enriched composition, in the presence of a cryoprotectant, thereby producing a cryopreserved composition.

29. The method of claim 28, wherein, prior to the incubating, washing the cryopreserved composition under conditions to reduce or remove the cyroprotectant and/or to generate the input composition.

30. The method of any of claims 1-29, wherein the input composition comprises N-acetylcysteine (NAC); serum, optionally human serum; recombinant interleukin-2 (IL-2), recombinant interleukin-15 (IL-15), and/or recombinant interleukin-7 (IL-7).

31. The method of any of claims 1-30, wherein:
the input composition comprises N-acetylcysteine at a concentration from or from about 0.4 mg/mL to 4 mg/mL, 0.8 mg/mL to 3.6 mg/mL or 1.6 mg/mL to 2.4 mg/mL, each inclusive; or
the input composition comprises N-acetylcysteine at a concentration of at least or at least about or about 0.4 mg/mL, 0.8 mg/mL, 1.2 mg/mL, 1.6 mg/mL, 2.0 mg/mL, 2.4 mg/mL, 2.8 mg/mL, 3.2 mg/mL, 3.6 mg/mL or 4.0 mg/mL.

32. The method of any of claims 1-31, wherein:
the input composition comprises serum, optionally human serum, at a concentration from or from about 0.5% to 25% (v/v), 1.0% to 10% (v/v) or 2.5% to 5.0% (v/v), each inclusive; or
the input composition comprises serum, optionally human serum, at a concentration of at least or at least about or about 0.5%, 1.%, 2.5%, 5% (v/v) or 10%.

33. The method of any of claims 1-32, wherein:
the input composition comprises recombinant IL-2, optionally recombinant human IL-2, at a concentration from or from about 10 IU/mL to 500 IU/mL, 50 IU/mL to 250 IU/mL or 100 IU/mL to 200 IU/mL, each inclusive; or at a concentration of at least or at least about 10 IU/mL, 50 IU/mL, 100 IU/mL, 200 IU/mL, 300 IU/mL, 400 IU/mL or 500 IU/mL; and/or
the input composition comprises recombinant IL-15, optionally recombinant human IL-15, at a concentration from or from about 1 IU/mL to 100 IU/mL, 2 IU/mL to 50 IU/mL or 5 IU/mL to 10 IU/mL, each inclusive; or at a concentration of at least or at least about 1 IU/mL, 2 IU/mL, 5 IU/mL, 10 IU/mL, 25 IU/mL or 50 IU/mL; and/or
the input composition comprises recombinant IL-7, optionally recombinant human IL-7, at a concentration from or from about 50 IU/mL to 1500 IU/mL, 100 IU/mL to 1000 IU/mL to 200 IU/mL to 600 IU/mL, each inclusive; or at a concentration of at least or at least about 50 IU/mL, 100 IU/mL, 200 IU/mL, 300 IU/mL, 400 IU/mL, 500 IU/mL, 600 IU/mL, 700 IU/mL, 800 IU/mL, 900 IU/mL or 1000 IU/mL.

34. The method of any of claims 1-33, wherein the incubating comprises a step of spinoculating the viral vector particles with the input composition.

35. The method of claim 34, wherein spinoculating comprises rotating, in an internal cavity of a centrifugal chamber, the viral vector particles and input composition, wherein the rotation is at a relative centrifugal force at an internal surface of the side wall of the cavity that is:
between or between about 500 g and 2500 g, 500 g and 2000 g 500 g and 1600 g, 500 g an 1000 g, 600 g and 1600 g, 600 g and 1000 g, 1000 g and 2000 g or 1000 g and 1600 g, each inclusive; or
at least or at least about 600 g, 800 g, 1000 g, 1200 g, 1600 g, or 2000 g.

36. The method of claim 34 or claim 35, wherein spinoculating is for a time that is:
greater than or about 5 minutes, greater than or about 10 minutes, greater than or about 15 minutes, greater than or about 20 minutes, greater than or about 30 minutes, greater than or about 45 minutes, greater than or about 60 minutes, greater than or about 90 minutes or greater than or about 120 minutes; or
between or between about 5 minutes and 60 minutes, 10 minutes and 60 minutes, 15 minutes and 60 minutes, 15 minutes and 45 minutes, 30 minutes and 60 minutes or 45 minutes and 60 minutes, each inclusive.

37. The method of any of claims 1-36, further comprising contacting the input composition and/or viral vector particles with a transduction adjuvant.

38. The method of claim 37, wherein the contacting is carried out prior to, concomitant with or after spinoculating the viral vector particles with the input composition.

39. The method of any of claims 1-38, wherein at least a portion of the incubation is carried out at or about 37° C.±2° C.

40. The method of any of claims 34-39, wherein the at least a portion of the incubation is carried out after the spinoculation.

41. The method of claim 39 or claim 40, wherein the at least a portion of the incubation is carried out for no more than or no more than about 2 hours, 4 hours, 12 hours, 18 hours, 24 hours, 30 hours, 36 hours, 48 hours, 60 hours or 72 hours.

42. The method of any of claims 39-41, wherein the at least a portion of the incubation is carried out for or for about 24 hours.

43. The method of any of claims 1-42, wherein the total duration of the incubation is for no more than 12 hours, 24 hours, 36 hours, 48 hours or 72 hours.

44. The method of any of claims 1-43, wherein the viral vector particle is a lentiviral vector particle.

45. The method of claim 44, wherein the lentiviral vector particle is derived from HIV-1.

46. The method of any of claims 1-45, wherein the viral vector particle is pseudotyped with a viral envelope glycoprotein.

47. The method of claim 46, wherein the viral envelope glycoprotein is VSV-G.

48. The method of any of claims 1-47, wherein the viral vector particle comprises a lentiviral protein that exhibits SAMHD1-inhibiting activity, said protein being packaged in the viral particle.

49. The method of claim 48, wherein the SAMHD1-inhibiting protein is a wild-type Vpx protein, a wild-type Vpr protein, or is a variant or portion of a wild-type Vpx or Vpr protein that exhibits SAMHD1-inhibiting activity.

50. The method of claim 48 or claim 49, wherein the SAMHD1-inhibiting protein is heterologous to the retroviral vector particle.

51. The method of any of claims 48-50, wherein the SAMHD1-inhibiting protein is a wild-type Vpx protein or is a variant or portion of a wild-type Vpx protein that exhibits SAMHD1-inhibiting activity.

52. The method of any of claims 1-51, wherein the viral vector particle is incubated at a multiplicity of infection of less than or less than about 20.0 or less than or less than about 10.0.

53. The method of any of claims 1-52, wherein:
the viral vector particle is incubated at a multiplicity of infection from or from about 1.0 IU/cell to 10 IU/cell or 2.0 U/cell to 5.0 IU/cell; or
the viral vector particle is incubated at a multiplicity of infection of at least or at least about 1.6 IU/cell, 1.8 IU/cell, 2.0 IU/cell, 2.4 IU/cell, 2.8 IU/cell, 3.2 IU/cell or 3.6 IU/cell, 4.0 IU/cell, 5.0 IU/cell, 6.0 IU/cell, 7.0 IU/cell, 8.0 IU/cell, 9.0 IU/cell or 10.0 IU/cell.

54. The method of any of claims 1-53, wherein the input composition comprises at least at or about at least or about 50×10⁶ cells, 100×10⁶ cells, or 200×10⁶ cells.

55. The method of any of claims 1-54, wherein the recombinant nucleic acid encodes an antigen receptor.

56. The method of claim 55, wherein the antigen receptor is a transgenic T cell receptor (TCR).

57. The method of claim 55 or claim 56, wherein the antigen receptor is a chimeric antigen receptor (CAR).

58. The method of claim 57, wherein the chimeric antigen receptor (CAR) comprises an extracellular antigen-recognition domain that specifically binds to a target antigen and an intracellular signaling domain comprising an ITAM.

59. The method of claim 58, wherein the intracellular signaling domain comprises an intracellular domain of a CD3-zeta (CD3) chain.

60. The method of claim 58 or claim 59, further comprising a transmembrane domain linking the extracellular domain and the intracellular signaling domain.

61. The method of claim 60, wherein the transmembrane domain comprises a transmembrane portion of CD28.

62. The method of any of claims 58-61, wherein the intracellular signaling domain further comprises an intracellular signaling domain of a T cell costimulatory molecule.

63. The method of claim 62, wherein the T cell costimulatory molecule is selected from the group consisting of CD28 and 41BB.

64. The method of any of claims 55-63, wherein the antigen receptor specifically binds to an antigen associated with a disease or condition or specifically binds to a universal tag.

65. The method of claim 64, wherein the disease or condition is a cancer, and autoimmune disease or disorder, or an infectious disease.

66. The method of any of claims 1-65, wherein the method produces an output composition comprising T cells transduced with the recombinant nucleic acid.

67. The method of claim 66, wherein at least 30%, or at least 40%, at least 50%, at least 60%, at least 70% or at least 80% of the T cells in the output composition are transduced with the recombinant nucleic acid.

68. The method of claim 66 or claim 67, further comprising recovering or isolating from the output composition the transduced T cells produced by the method.

69. The method of claim 66 or claim 68, further comprising activating or expanding the cells of the output composition or the cells transduced by the method.

70. The method of claim 69, wherein activation and/or expansion is performed ex vivo.

71. The method of claim 69 or claim 70, wherein, subsequent to the incubation, the cells in the output composition are further incubated in the presence of one or more stimulating agent capable of activating T cells, inducing a signal through a TCR complex and/or inducing proliferation of T cells.

72. The method of claim 71, wherein the one or more stimulating agent is selected from the group consisting of CD3-binding molecules; CD28-binding molecules; recombinant IL-2; recombinant IL-15; and recombinant IL-7.

73. The method of claim 71 or claim 72, wherein the one or more stimulating agents comprise an anti-CD3 antibody and/or an anti-CD28 antibody.

74. The method of claim 69, wherein activation and/or expansion is performed in vivo.

75. The method of claim 69 or claim 74, wherein activation and/or expansion occurs in the presence of antigen specifically bound by the antigen receptor and/or is transgene-specific.

76. The method of claim 72 or claim 75, wherein, subsequent to the incubation, the cells in the output composition are not further incubated ex vivo in the presence of one or more stimulating agent, optionally consisting of CD3-binding molecules; CD28-binding molecules; recombinant IL-2; recombinant IL-15; and recombinant IL-7, and/or the cells in the output composition are not further incubated at a temperature greater than 30° C. for more than 24 hours.

77. A genetically engineered T cell produced by the method of any of claims 1-76.

78. A composition, comprising the genetically engineered T cell of claim 77 and a pharmaceutically acceptable carrier.

79. A method of treatment, the method comprising administering to a subject having a disease or condition the composition of claim 78.

80. The method of claim 79, wherein the composition is administered to the subject no more than 5 days after obtaining the sample from the subject.

81. The method of claim 80, wherein the composition is administered to the subject no more than 1 day, 2 days, 3 days or 4 days after obtaining the sample from the subject.

82. A method for adoptive cell therapy, comprising:
(a) enriching or isolating T cells from a sample obtained from subject having a disease or condition;
(b) transducing an input composition comprising the T cells with a viral vector particle by the method of any of claims 1-71, thereby producing an output composition comprising transduced cells, wherein the viral vector particle comprises a recombinant nucleic acid encoding an antigen receptor that specifically binds to an antigen associated with the disease or disorder;
(c) administering the output composition comprising the transduced cells to the subject for treating the disease or condition, wherein the output composition is administered to the subject no more than 9 days after obtaining the sample from the subject.

83. A method of adoptive cell therapy, comprising administering an output composition comprising T cells transduced with a recombinant nucleic acid to a subject for treating a disease or condition, wherein the output composition is produced by a method of any of claims 1-71.

84. The method of claim 82 or claim 83, wherein the output composition is administered to the subject no more than 1 day, 2 days, 3 days, 4 days or 5 days after obtaining the sample from the subject.

85. The method of any of claims 82-84, wherein prior to administering the composition, the transduced cells or the cells of the output composition are formulated in a pharmaceutically acceptable buffer.

86. The method of any of claims 82-85, wherein, prior to administering the output composition comprising transduced cells, the cells are cultured ex vivo for up to 24 hours, 2 days, 3 days, 4 days, 5 days, 6 days, 7 days, 8 days or 9 days after the transducing, said culturing occurring at a temperature greater than 30° C.

87. The method of any of claims 82-86, wherein, subsequent to the transduction, the output composition or cells comprising the transduced cells are cultured in the presence of one or more stimulating agent capable of activating T cells, inducing a signal through a TCR complex and/or inducing proliferation of T cells, thereby producing the composition comprising the transduced cells.

88. The method of any of claims 82-85, wherein, prior to administering the transduced cells, the output composition or cells comprising the transduced cells are not further incubated ex vivo in the presence of one or more stimulating agent and/or are not further incubated at a temperature greater than 30° C. for more than 24 hours.

89. The method of claim 87 or claim 88, wherein the one or more stimulating agent is selected from the group consisting of CD3-binding molecules; CD28-binding molecules; recombinant IL-2; recombinant IL-15; and recombinant IL-7, a vaccine comprising an antigen specifically recognized by the antigen receptor, and an anti-idiotype antibody that specifically binds the antigen receptor.

90. The method of claim 89, wherein the one or more stimulating agents comprise an anti-CD3 antibody and/or an anti-CD28 antibody.

91. The method of any of claims 82-90, wherein the cells of the output composition or the transduced cells are administered at a sub-optimal dose.

92. The method of any of claims 82-91, further comprising administering to the subject one or more agents to induce or enhance stimulation and/or expansion of the transduced T cells in vivo.

93. The method of claim 92, wherein the one or more agents is transgene-specific and/or stimulates or activates the cells via the expressed transgene, which optionally is or comprises an antigen receptor.

94. The method of claim 92 or claim 93, wherein the one or more agent is selected from among a vaccine comprising an antigen specifically recognized by the antigen receptor, an anti-idiotype antibody that specifically binds the antigen receptor or an agent capable of chemically inducing dimerization of the antigen receptor.

95. The method of claim 92, wherein the one or more agents is an immunomodulatory agent; an immune checkpoint inhibitor; an inhibitor of extracellular adenosine or adenosine receptor, optionally an A2aR receptor; a kynurenine pathway modulator, and modulators of signaling pathways, e.g., kinase inhibitors.

96. A composition, comprising a population of primary human T cells genetically engineered to express a chimeric antigen receptor (CAR) or transgenic TCR that specifically binds to a target antigen, wherein:
the population comprises a plurality of resting T cells; and
the plurality of resting T cells comprise at least 7.5% percent of the genetically engineered cells in the composition.

97. The composition of claim 96, wherein the genetically engineered resting T cells comprise at least 10%, at least 20%, at least 30%, at least 40%, at least 50%, at least 60%, at least 70%, at least 80%, or at least 90% of the genetically engineered cells in the composition.

98. The composition of claim 96 or claim 97, wherein the resting T cells are surface negative for a T cell activation marker selected from the group consisting of HLA-DR, CD25, CD69, CD71, CD40L (CD154) and 4-1BB (CD137); lack intracellular expression of a cytokine selected from the group consisting of IL-2, IFN-gamma and TNF-alpha; are in the G0 or G₀G₁ₐ stage of the cell cycle; and/or contain an active SAMHD1.

99. The composition of claim 98, wherein the resting T cells are surface negative for CD25 and CD69 (CD25-/CD69-).

100. The composition of any of claims 96-99, wherein the resting T cells comprise CD4+ and/or CD8+ T cells.

101. The composition of any of claims 96-100, wherein the target antigen is associated with a disease or disorder.

102. The composition of claim 100, wherein the disease or disorder is an infectious disease or condition, an autoimmune disease, an inflammatory disease or a cancer.

103. The composition of any of claims 96-102, wherein the target antigen is selected from the group consisting of B cell maturation antigen (BCMA), carbonic anhydrase 9 (CAIX), tEGFR, Her2/neu (receptor tyrosine kinase erbB2), CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR viii, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L-cell adhesion molecule (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE A1, HLA-A2, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, G Protein Coupled Receptor 5D (GPCR5D), ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag.

104. The composition of any of claims 96-103, wherein the primary human T cells are genetically engineered to express a CAR, which comprises an extracellular antigen-recognition domain that specifically binds to a target antigen and an intracellular signaling domain comprising an ITAM.

105. The composition of claim 104, wherein the intracellular signaling domain comprises an intracellular domain of a CD3-zeta (CD3) chain.

106. The composition of claim 104 or claim 105, wherein the CAR further comprises a transmembrane domain linking the extracellular domain and the intracellular signaling domain.

107. The composition of claim 106, wherein the transmembrane domain comprises a transmembrane portion of CD28.

108. The composition of any of claims 104-107, wherein the intracellular signaling domain of the CAR further comprises an intracellular signaling domain of a T cell costimulatory molecule.

109. The composition of claim 108, wherein the T cell costimulatory molecule is selected from the group consisting of CD28 and 41BB.

110. The composition of any of claims 96-108, comprising a pharmaceutically acceptable carrier.
